(19) United States
(12) Patent Application Publication
(10) Pub. No.: US 2017/0101468 A1
(43) Pub. Date: Apr. 13, 2017

(54) HUMAN ANTI IL-6 ANTIBODIES WITH EXTENDED IN VIVO HALF-LIFE AND THEIR USE IN TREATMENT OF ONCOLOGY, AUTOIMMUNE DISEASES AND INFLAMMATORY DISEASES

(71) Applicant: MedImmune Limited, Cambridge (GB)

(72) Inventors: Michael Bowen, Gaithersburg, MD (US); Herren Wu, Gaithersburg, MD (US); William Dall'Acqua, Gaithersburg, MD (US); Peter Kiener, Potomac, MD (US); Bahija Jallal, Gaithersburg, MD (US); Anthony Coyle, Boston, MA (US)

(21) Appl. No.: 15/368,499

(22) Filed: Dec. 2, 2016

Related U.S. Application Data
(63) Continuation of application No. 14/253,161, filed on Apr. 15, 2014.
Application No. 14/253,161 is a continuation of application No. 13/146,278, filed on Oct. 20, 2011, now abandoned.
(60) Provisional application No. 61/184,182, filed on Jun. 4, 2009.
(60) Provisional application No. 61/148,106, filed on Jan. 29, 2009.

(51) Int. Cl. C07K 16/24

(52) U.S. Cl. CPC C07K 16/248; C07K 2317/21; C07K 2317/94; C07K 2317/92; C07K 2317/52; C07K 2317/72; A61K 2039/505

(57) ABSTRACT

The present invention provides human anti-IL-6 antibodies with extended in vivo half-life. The invention further relates to pharmaceutical compositions, therapeutic compositions, and methods using therapeutic antibodies that bind to IL-6 and that has an extended in vivo half-life for the treatment and prevention of IL-6 mediated diseases and disorders, such as, but not limited to, inflammatory diseases and disorders, autoimmune diseases and disorders and tumors.

### STATEMENT OF PRIORITY

This application is a continuation of co-pending U.S. application Ser. No. 14/253,161, filed on Apr. 15, 2014, which is a continuation of Ser. No. 13/146,278 filed on Oct. 20, 2011, which application is the National Stage of International Application No. PCT/US2010/022478, filed Jan. 29, 2010, said International Application No. PCT/US2010/022478 claims the benefit of U.S. Provisional Application No. 61/184,182, filed Jun. 4, 2009, and U.S. Provisional Application No. 61/148,106, filed Jan. 29, 2009. Each of which is hereby incorporated in its entirety by reference.

### FIELD OF THE INVENTION

This invention relates to anti-IL-6 antibody molecules that inhibit biological effects of IL-6 and have an extended in vivo half-life. The anti-IL-6 antibodies are useful for treatment of disorders associated with IL-6, including inflammatory disorders, autoimmune disorders, tumors and depression.

### BACKGROUND

Interleukin 6 (IL-6) is a 26 kDa pleiotropic pro-inflammatory cytokine produced by a variety of cell types, including stimulated fibroblasts, monocytes and endothelial cells, which form the major source of IL-6 in vivo. Cells such as T cells, B cells, macrophages, keratinocytes, osteoblasts and several others can produce IL-6 on stimulation. IL-6 is also expressed from tumor cell lines and tumor cells e.g. cells from lung carcinoma, prostate cancer, myeloma, hypernephroma and cardiac myxoma (Kishimoto, T., (1989) Blood 74:1-10; Smith P. C. et al. (2001) Cytokine and Growth factor Reviews 12:33-40). Under non-inflammatory conditions, IL-6 is secreted from adipose tissue (Wallenius et al., (2002) Nat. Med. 8:75).

To initiate cell signalling, IL-6 binds with low affinity to a transmembrane receptor, IL-6 receptor alpha (also referred to as IL-6Rα, IL-6Ra, IL-6R, gp80 or CD126) to form a complex “IL-6:IL-6Ra”. This complex binds to the gp130 signal receptor; IL-6Rα and gp130 together form a high affinity IL-6 binding site, and induce the formation of a hexamer composed of two copies each of IL-6, IL-6Ra and gp130 (Somers, W., et al (1997) 1.9 EMBO J. 16:989-997). The transmembrane and cytoplasmic domains of the IL-6Ra are not required for signal transduction, as IL-6Ra also exists as a soluble secreted form (sIL-6R or sIL-6Ra). The soluble receptor is produced either by differential splicing of the IL-6Ra message or by proteolytic shedding. sIL-6R is capable of forming a ligand-receptor complex with IL-6, “IL-6:sIL-6Ra”. This complex can bind gp130 on cells and thereby initiate cell signalling in gp130 positive cells, even if those cells do not express IL-6Ra. Thus, sIL-6R has the potential to widen the repertoire of cells responsive to IL-6, and is thought to play an important role in IL-6-mediated inflammation (Jones, S. A et al. (2001) FASEB J. 15:43-58).

A crystal structure of human IL-6 ligand has been elucidated (Somers, W., et al (1997) 1.9 EMBO J. 16:989-997). The crystal structure of the extracellular domain of human IL-6Ra (Varghese et al. (2002) PNAS USA 99:15959-15964), and the hexameric structure of IL-6/IL-6R/gp130 complex (Boulanger et al (2003) Science 300:2101-2104), have also been resolved. These structures combined with mutagenesis studies have identified three sites on the surface of IL-6 which are involved in the functional activity of the IL-6 in complex with the various receptor components. Site 1 residues are involved in the interaction between IL-6 and IL-6Ra. Site 2 residues are involved in the interaction between IL-6 and the gp130 cytokine binding domain. The residues in Site 3 of IL-6 are involved in interacting with the Ig-like domain of the second gp130 in the hexameric complex. A fourth site on IL-6 has also been identified where IL-6 interacts with the second molecule of IL-6 in the hexameric IL-6/IL-6R/gp130 complex (Menziani et al (1997) Proteins: Structure Function and Genetics 29, 528).

A number of anti-IL-6 ligand monoclonal antibodies have been isolated. Mapping studies have been performed which show that these bind to different binding sites, as described above, on the surface of human IL-6 (Brakenhoff et al. (1990) J. Immunol. 145:561-568; Wijdenes et al. (1991) Mol Immunol. 28:1183-1191; Brakenhoff et al. (1994) JBC 269:86; Kalai et al. (1996) Eur J Biochem 238 714-723; Kalai et al. (1997) Blood 89:1319-1333).

The elevation of IL-6 has been implicated as a key cytokine in a variety of disease indications. The levels of circulating IL-6 have been shown to be elevated in diseases such as rheumatoid arthritis, Castleman's disease, Juvenile idiopathic arthritis and Crohn's Disease (Nishimoto N, and Kishimoto T. (2004) Curr Op in Pharmacology 4:386-391). Because of this IL-6 has been implicated in driving the pathology in these inflammatory indications. Furthermore, a variety of tumor types have been shown to be stimulated by IL-6, including melanoma, renal cell carcinoma, Kaposi's sarcoma, ovarian carcinoma, lymphoma, leukaemia, multiple myeloma, and prostate carcinoma (Keller E. T. et al. (1996) Front Biosci. 1:340-57). Moreover increased circulating levels of IL-6 have been reported in several cancers. In some cancer indications elevated IL-6 levels has been used as prognostic indicators of the disease.

Because of the role of IL-6 in disease a variety of murine, chimeric, humanized and human anti-human IL-6 monoclonal antibodies have been developed as potential therapies (e.g., U55856135, WO2004/020633, US20060257407A1, U57291721). A chimeric human-mouse anti-IL-6 antibody cCLB8 (known as CNTO 328) has been used to treat patients with multiple myeloma (van Zaanen et al. (1998) Brit. Journal. Haematology 102:783), with disease stabilisation seen in the majority of patients.

The positive effect of inhibiting IL-6 signalling in cancer and inflammatory diseases has been further highlighted by the use of a humanized anti-IL-6Ra antibody Tocilizumab (also known as hPM-1, MRA and Actemra). This is a humanized version of the murine anti-IL6Ra antibody PM-1. Treatment of patients with this antibody has proven effective in a number of diseases including rheumatoid arthritis, juvenile idiopathic arthritis, Crohn's disease, myeloproliferative disorder, Castleman's disease and systemic lupus erythematosus (SLE) (Mihara et al. (2005) Expert Opinion on Biological Therapy. 5:683-90).

A critical issue in antibody based therapies is the persistence of immunoglobulins in the circulation. The rate of immunoglobulin clearance directly affects the amount and frequency of dosage of the immunoglobulin. Increased dosage and frequency of dosage may cause adverse effects in the patient and also increase medical costs. In view of the pharmaceutical importance of anti-IL-6 antibody based therapies, there is a need to develop modified high affinity human anti-IL-6 antibodies having an increased in vivo half-life.

### SUMMARY OF THE INVENTION

The present invention relates to high affinity human anti-IL-6 antibodies that specifically bind human IL-6 and have an extended in vivo half-life. In one embodiment, the in vivo half-life of an anti-IL-6 antibody described herein is between 10 days and 40 days. In a specific embodiment, the in vivo half-life of an anti-IL-6 antibody described herein is between 25 days and 35 days. In one embodiment, an anti-IL-6 antibody described herein comprises the VH and/or VL domain of an anti-IL-6 antibody described in PCT Publication No. WO 2008/065378. In one embodiment, an anti-IL-6 antibody of the invention comprises a human IgG constant domain having one or more amino acid substitutions relative to a wild-type human IgG constant domain. In a specific embodiment, an anti-IL-6 antibody of the invention comprises a human IgG constant domain having the M252Y, S254T, and T256E amino acid substitutions, wherein amino acid residues are numbered according to the EU index as in Kabat. In another embodiment, an anti-IL-6 antibody of the invention comprises a heavy chain sequence of SEQ ID NO:9 and a light chain sequence of SEQ ID NO:10.

The present invention further relates to nucleic acids encoding a human anti-IL-6 antibody having an extended half-life, vectors comprising the nucleic acids, cells comprising the vectors and methods of making a human anti-IL-6 antibody having an extended half-life.

In further aspects, the invention provides an isolated nucleic acid which comprises a sequence encoding a human anti-IL-6 antibody having an extended half-life according to the present invention, and methods of preparing a human anti-IL-6 antibody having an extended half-life, which comprise expressing said nucleic acid under conditions to bring about production of said human anti-IL-6 antibody, and recovering it.

A further aspect provides a host cell containing or transformed with nucleic acid of the invention.

Further aspects of the present invention provide for compositions comprising an anti-IL-6 antibody of the invention, and their use in methods of binding, inhibiting and/or neutralising IL-6, including methods of treatment of the human or animal body by therapy. In one embodiment, a composition of the invention is a sterile, liquid formulation. In a specific embodiment, a composition of the invention comprises at least 100 mg/ml of an anti-IL-6 antibody of the invention. In another embodiment, a composition of the invention is a lyophilized formulation. In a further embodiment, a formulation of the invention is a pharmaceutical formulation.

Antibodies according to the invention may be used in a method of treatment or diagnosis, such as a method of treatment (which may include prophylactic treatment) of a disease or disorder in the human or animal body (e.g. in a human patient), which comprises administering to said patient an effective amount of a binding member of the invention. Conditions treatable in accordance with the present invention include any in which IL-6 plays a role, as discussed in detail elsewhere herein.

The present invention also encompasses methods of neutralizing IL-6 activity in the serum of a human patient in need thereof, comprising administering to the human patient an effective amount of an anti-IL-6 antibody of the invention. The present invention further provides methods of preventing, managing, treating or ameliorating an inflammatory disease or disorder, an autoimmune disease or disorder, a proliferative disease, a disease or disorder associated with or characterized by aberrant expression and/or activity of IL-6, a disease or disorder associated with or characterized by aberrant expression and/or activity of the IL-6 receptor, or one or more symptoms thereof, said methods comprising administering to a subject in need thereof a prophylactically or therapeutically effective amount of an anti-IL-6 antibody of the invention.

One aspect of the invention relates to an isolated modified antibody that specifically binds to IL-6, wherein the modified antibody comprises a variable domain and a human IgG constant domain having one or more amino acid substitutions relative to a wild-type human IgG constant domain, wherein the antibody has an increased half-life compared to the half-life of a parent antibody comprising said variable domain and the wild-type human IgG constant domain. In one embodiment of this aspect of the invention, the half-life of the modified antibody is at least 2 times, at least 3 times, at least 4 times, at least 5 times, at least 10 times or at least 20 times longer than the half-life of the wild type antibody. In another embodiment, the half-life of the modified antibody is 2 times, 3 times, 4 times, 5 times, 10 times or 20 times longer than the half-life of the wild type antibody. In a further embodiment, the half-life of the modified antibody is between 2 times and 3 times, between 2 times and 5 times, between 2 times and 10 times, between 3 times and 5 times, or between 3 times and 10 times longer than the half-life of the wild type antibody. In still another embodiment, the half-life of the modified antibody is at least 10 days, at least 15 days, at least 20 days, at least 25 days, at least 26 days, at least 27 days, at least 28 days, at least 29 days, at least 30 days, at least 35 days, at least 40 days, at least 45 days or at least 50 days. In still a further embodiment, the half-life of the modified antibody is 10 days, 15 days, 20 days, 25 days, 26 days, 27 days, 28 days, 29 days, 30 days, 35 days, 40 days, 45 days or 50 days. In still a further embodiment, the half-life of the modified antibody is between 10 days and 20 days, between 10 days and 30 days, between 10 days and 40 days, between 10 days and 50 days, between 20 days and 30 days, between 20 days and 40 days, between 20 days and 50 days, between 25 days and 30 days, between 25 days and 40 days, between 25 days and 50 days, between 30 days and 40 days, between 30 days and 50 days or between 40 days and 50 days. In yet a further embodiment, the half-life of the modified antibody is the half-life measured in a mammal. In another embodiment, the half-life of the modified antibody is the half-life measured in non-human primate. In a further embodiment, the modified antibody is the half-life measured in a human subject.

Another aspect of the invention relates to an isolated modified antibody that specifically binds to IL-6, wherein the modified antibody comprises a human IgG constant domain having one or more amino acid substitutions relative to a wild-type human IgG constant domain, wherein the antibody has a decreased clearance rate compared to the clearance rate of a wild-type antibody comprising the wild-type human IgG constant domain. In one embodiment of this aspect of the invention, the clearance rate of the modified antibody is at least 2 times, at least 3 times, at least 4 times, at least 5 times, at least 10 times or at least 20 times lower than the clearance rate of the wild type antibody. In another embodiment, the clearance rate of the modified antibody is 2 times, 3 times, 4 times, 5 times, 10 times or 20 times lower than the clearance rate of the wild type antibody. In still a further embodiment, the clearance rate of the modified antibody is between 2 times and 3 times, between 2 times and 5 times, between 2 times and 10 times, between 3 times and 5 times, or between 3 times and 10 times lower than the clearance rate of the wild type antibody. In another embodiment, the clearance rate of the modified antibody is at most 1 mL/kg/day, at most 2 mL/kg/day, at most 3 mL/kg/day, at most 4 mL/kg/day, at most 5 mL/kg/day, at most 7 mL/kg/day, at most 10 mL/kg/day, at most 15 mL/kg/day or at most 20 mL/kg/day. In a further embodiment, the clearance rate of the modified antibody is 1 mL/kg/day, 2 mL/kg/day, 3 mL/kg/day, 4 mL/kg/day, 5 mL/kg/day, 7 mL/kg/day, 10 mL/kg/day, 15 mL/kg/day or 20 mL/kg/day. In still another embodiment, the clearance rate of the modified antibody is between 1 mL/kg/day and 2 mL/kg/day, between 1 mL/kg/day and 3 mL/kg/day, between 1 mL/kg/day and 5 mL/kg/day, between 1 mL/kg/day and 10 mL/kg/day, between 1 mL/kg/day and 15 mL/kg/day, between 2 mL/kg/day and 5 mL/kg/day, between 2 mL/kg/day and 10 mL/kg/day, between 3 mL/kg/day and 5 mL/kg/day, between 3 mL/kg/day and 10 mL/kg/day or between 5 mL/kg/day and 10 mL/kg/day. In yet a further embodiment, the clearance rate of the modified antibody is the clearance rate measured in a mammal. In another embodiment, the modified antibody is the clearance rate measured in non-human primate. In a further embodiment, the clearance rate of the modified antibody is the clearance rate measured in a human subject. In yet a further embodiment, the amino acid substitutions are selected from the group consisting of: M252Y, M252F, M252W, M252T, S254T, T256S, T256R, T256Q, T256E, T256D, T256T, L309P, Q311S, H433R, H433K, H433S, H433I, H433P, H433Q, N434H, N434F, N434Y and N436H, wherein amino acid residues are numbered according to the EU index as in Kabat. In another embodiment, at least one of the amino acid substitutions is selected from the group consisting of: M252Y, S254T, T256E, H433K, N434F and N436H, wherein amino acid residues are numbered according to the EU index as in Kabat. In still another embodiment, the modified IgG constant domain comprises the M252Y, S254T, and T256E amino acid substitutions, wherein amino acid residues are numbered according to the EU index as in Kabat. In yet another embodiment, the modified IgG constant domain has a higher affinity for FcRn than the wild-type IgG constant domain. In a further embodiment, the human IgG constant domain is a human IgG1, IgG2, IgG3 or IgG4 constant domain. In still a further embodiment, the IgG is IgG1.

Another aspect of the invention relates to the modified antibodies described above wherein the variable domain comprises: a VH CDR1 having an amino acid sequence identical to or comprising 1, 2, or 3 amino acid residue substitutions relative to SEQ ID NO: 1; a VH CDR2 having an amino acid sequence identical to or comprising 1, 2, or 3 amino acid residue substitutions relative to SEQ ID NO: 2; a VH CDR3 having an amino acid sequence identical to or comprising 1, 2, or 3 amino acid residue substitutions relative to SEQ ID NO: 3; a VL CDR1 having an amino acid sequence identical to or comprising 1, 2, or 3 amino acid residue substitutions relative to SEQ ID NO: 4; VL CDR2 having an amino acid sequence identical to or comprising 1, 2, or 3 amino acid residue substitutions relative to SEQ ID NO: 5; and a VL CDR3 having an amino acid sequence identical to or comprising 1, 2, or 3 amino acid residue substitutions relative to SEQ ID NO: 6. In one embodiment, the modified antibody of any one of claims claim 1-26, wherein the variable domain comprises: a VH CDR1 having the amino acid sequence of SEQ ID NO: 1; a VH CDR2 having the amino acid sequence of SEQ ID NO: 2; a VH CDR3 having the amino acid sequence of SEQ ID NO: 3; a VL CDR1 having the amino acid sequence of SEQ ID NO: 4; a VL CDR2 having the amino acid sequence of SEQ ID NO: 5; and a VL CDR3 having the amino acid sequence of SEQ ID NO: 6. In another embodiment, the variable domain comprises a VH domain comprising three CDRs and a VL domain comprising three CDRs; wherein the three CDRs of the VH domain comprise: a VH CDR1 comprising the amino acid sequence of SEQ ID NO: 1; a VH CDR2 comprising the amino acid sequence of SEQ ID NO: 2; and a VH CDR3 comprising the amino acid sequence of SEQ ID NO: 3. In a further embodiment, the variable domain comprises a VH domain comprising three CDRs and a VL domain comprising three CDRs, wherein the three CDRs of the VL domain comprise: a VL CDR1 comprising the amino acid sequence of SEQ ID NO: 4; a VL CDR2 comprising the amino acid sequence of SEQ ID NO: 5; and a VL CDR3 comprising the amino acid sequence of SEQ ID NO: 6. In yet a further embodiment, the variable domain comprises a VH domain having an amino acid sequence identical to SEQ ID NO: 7 or comprising 1, 2, 3, 4, 5, 6, 7, 8, 9, or 10 amino acid residue substitutions relative to SEQ ID NO: 7 and comprises a VL domain having an amino acid sequence identical to SEQ ID NO:8 or comprising 1, 2, 3, 4, 5, 6, 7, 8, 9, or 10 amino acid residue substitutions relative to SEQ ID NO:8. In another embodiment, the variable domain comprises the VH domain of SEQ ID NO:7 and the VL domain of SEQ ID NO:8.

Another aspect of the invention relates to a nucleic acid encoding the amino acid sequence encoding the aforementioned modified antibodies. In one embodiment, the nucleic acid comprises a nucleotide sequence selected from the group consisting of SEQ ID NO:11-14.

Another aspect of the invention relates to a vector comprising the aforementioned nucleic acids

Another aspect of the invention relates to an isolated cell comprising the aforementioned vectors.

Another aspect of the invention relates to an isolated cell expressing the aforementioned modified antibodies.

Another aspect of the invention relates to a method of producing a modified antibody comprising culturing the aforementioned isolated cells under conditions sufficient for the production of the antibody and recovering the antibody from the culture.

Another aspect of the invention relates to a pharmaceutical composition comprising the aforementioned modified antibodies.

Another aspect of the invention, relates to a method of neutralizing at least 90% of the free IL-6 in the serum of a human in need thereof, comprising administering an effective amount of the aforementioned modified antibodies.

Another aspect of the invention relates to a method of inhibiting at least 90% of IL-6 mediated signaling in the serum of a human in need thereof, comprising administering to the human an effective amount of the aforementioned modified antibodies.

Another aspect of the invention relates to a method of neutralizing at least 90% of the free IL-6 in the synovial fluid of a human in need thereof, comprising administering to the human an effective amount of the modified antibody.

Another aspect of the invention relates to a method of inhibiting at least 90% of IL-6 mediated signaling in the synovial fluid of a human in need thereof, comprising administering to the human an effective amount of the aforementioned antibodies.

Another aspect of the invention relates to a method of reducing synovial cell growth in a human comprising administering to a human in need thereof a therapeutically effective amount of the aforementioned antibodies.

Another aspect of the invention relates to a method of reducing synovial inflammation in a human comprising administering to a human in need thereof a therapeutically effective amount of the aforementioned antibodies.

Another aspect of the invention relates to a method of treating an autoimmune disease or disorder in a human comprising administering to a human in need thereof a therapeutically effective amount of the aforementioned antibodies.

Another aspect of the invention relates to a method of treating a malignancy in a human comprising administering to a human in need thereof a therapeutically effective amount of the aforementioned antibodies.

Another aspect of the invention relates to a method of treating an inflammatory disease or disorder in a human comprising administering to a human in need thereof a therapeutically effective amount of the aforementioned antibodies.

Another aspect of the invention method of treating systemic lupus erythematosus, rheumatoid arthritis or inflammatory bowel disease in a human comprising administering to a human in need thereof a therapeutically effective amount of the aforementioned antibodies. In on the embodiment, the method of any one of claims 40-49, wherein the therapeutically effective amount comprises a single or divided dose of about 0.1-5 mg/kg, about 0.1-2 mg/kg, about 0.1-1 mg/kg, about 0.3-2 mg/kg, about 0.3-1 mg/kg, about 0.5-2 mg/kg, or about 0.5-1 mg/kg modified antibody. In another embodiment, the therapeutically effective amount comprises a single or divided dose of about 20-500 mg, about 20-200 mg, about 20-100 mg, about 50-500 mg, about 50-200 mg, or about 50-100 mg modified antibody. In yet another embodiment, the therapeutically effective amount of modified antibody is administered once a week, once every two weeks, once every three weeks, once every four weeks, once every eight weeks or once every twelve weeks. In still a further embodiment, the therapeutically effective amount of modified antibody is administered intravenously or subcutaneously. In another embodiment, the patient is administered a single loading dose of modified antibody before being administered at least one maintenance dose of the modified antibody. In still a further embodiment, the loading dose comprises a single or divided dose of about 0.1-5 mg/kg, about 0.1-2 mg/kg, about 0.1-1 mg/kg, about 0.3-2 mg/kg, about 0.3-1 mg/kg, about 0.5-2 mg/kg, or about 0.5-1 mg/kg modified antibody. In still a further embodiment, the loading dose comprises a single or divided dose of about 20-500 mg, about 20-200 mg, about 20-100 mg, about 50-500 mg, about 50-200 mg, or about 50-100 mg modified antibody. In still another embodiment, the maintenance dose comprises a single or divided dose of about 0.1-5 mg/kg, about 0.1-2 mg/kg, about 0.1-1 mg/kg, about 0.3-2 mg/kg, about 0.3-1 mg/kg, about 0.5-2 mg/kg, or about 0.5-1 mg/kg modified antibody. In yet another embodiment, the maintenance dose comprises a single or divided dose of about 20-500 mg, about 20-200 mg, about 20-100 mg, about 50-500 mg, about 50-200 mg, or about 50-100 mg modified antibody. In another embodiment, the maintenance dose is administered one week, two weeks, three weeks, four weeks, 8 weeks, or twelve weeks after administering the loading dose. In still another embodiment, the at least two maintenance doses are administered to the patient and the maintenance doses are administered once a week, once every two weeks, once every three weeks, once every four weeks, once every eight weeks or once every twelve weeks. In yet another embodiment, the loading dose of modified antibody is administered intravenously or subcutaneously. In another embodiment the maintenance dose is administered intravenously or subcutaneously. In a further embodiment, the therapeutically effective amount of the modified antibody is administered in conjunction with a second therapeutic agent.

Another aspect of the invention relates to a sterile, stable aqueous formulation comprising the aforementioned antibodies. In one embodiment of this aspect of the invention, the antibody was not subjected to lyophilization. In another embodiment, the antibody was subjected to lyophilization. In another embodiment, the concentration of said modified antibody is at least about 5 mg/ml, at least about 10 mg/ml, at least about 15 mg/ml, at least about 20 mg/ml, at least about 50 mg/ml, at least about 100 mg/ml, at least about 120 mg/ml, at least about 150 mg/ml, at least about 160 mg/ml, at least about 180 mg/ml, at least about 200 mg/ml, at least about 250 mg/ml, or at least about 300 mg/ml. In a further embodiment, the formulation further comprises at least about one buffering component. In another embodiment, the formulation further comprises at least one excipient. In still another embodiment, the buffering component is selected from the group consisting of histidine, citrate, phosphate, glycine, and acetate. In yet another embodiment, the buffering component is at a concentration from about 1 mM to about 200 mM, from about 1 mM to about 50 mM, or from about 5 mM to about 20 mM. In still another embodiment, the buffering component is at a concentration of about 10 mM, about 15 mM, about 20 mM or about 25 mM. In a further embodiment, the excipient is a saccharide. In still another embodiment, the saccharide is a disaccharide. In still another embodiment, the disaccharide is trehalose or sucrose. In a further embodiment, the disaccharide is at a concentration from about 1% to about 40%, from about 2% to about 20%, or from about 2% to about 10%. In still a further embodiment, the disaccharide is at a concentration of about 2%, about 4% or about 8%. In still another embodiment, the excipient is a salt. In yet another embodiment, the salt is sodium chloride. In a further embodiment, the sodium chloride is at a concentration from about 50 mM to about 200 mM. In another embodiment, the sodium chloride is at a concentration of about 70 mM, about 75 mM, about 80 mM, about 100 mM, about 120 mM, or about 150 mM. In a further embodiment, the excipient is a surfactant. In a further embodiment, the surfactant is a polysorbate. In still a further embodiment, the polysorbate is polysorbate 20 or polysorbate 80. In yet a further embodiment, the surfactant is at a concentration from about 0.001% to about 2%. In another embodiment, the surfactant is at a concentration of about 0.01%, about 0.02%, about 0.04% or about 0.08%. In still a further embodiment, the excipient is an amino acid. In still another embodiment, the amino acid is selected from the group consisting of glycine, histidine or arginine. In yet another embodiment, the amino acid is at a concentration of between about 10 mM and about 400 mM. In still another embodiment, the amino acid is at a concentration of about 25 mM, about 50 mM, about 100 mM, about 150 mM, about 200 mM, about 250 mM, about 300 mM, about 350 mM, or about 400 mM. In yet a further embodiment, the formulation has a pH of between about 5.5 and about 6.5. In still another embodiment, the said formulation has a pH of about 6.0. In still another embodiment the formulation is isotonic. In another embodiment the formulation is stable upon storage at 40° C. for at least about 4 weeks. In still another embodiment, the formulation is stable upon storage at 5° C. for at least about 3 months. In another embodiment the formulation is stable upon storage at 5° C. for at least about 12 months. In still another embodiment, the antibody loses at most 20% of its IL-6 binding activity during storage of said formulation at 40° C. for at least about 4 weeks. In yet a further embodiment, the antibody loses at most 20% of its IL-6 binding activity during storage of said formulation at 5° C. for at least about 3 months. In still another embodiment, the antibody loses at most 20% of its IL-6 binding activity during storage of said formulation at 5° C. for at least about 12 months. In still another embodiment, the antibody loses at most 10% of its IL-6 binding activity during storage of said formulation at 40° C. for at least about 4 weeks. In another embodiment, the formulation of any one of claims 64 to 93, wherein said antibody loses at most 10% of its IL-6 binding activity during storage of said formulation at 5° C. for at least about 3 months. In another embodiment, antibody loses at most 10% of its IL-6 binding activity during storage of said formulation at 5° C. for at least about 12 months. In another embodiment, the antibody loses at most 5% of its IL-6 binding activity during storage of said formulation at 40° C. for at least about 4 weeks. In another embodiment, the antibody loses at most 5% of its IL-6 binding activity during storage of said formulation at 5° C. for at least about 3 months. In another embodiment, the antibody loses at most 5% of its IL-6 binding activity during storage of said formulation at 5° C. for at least about 12 months. In another embodiment, the antibody is susceptible to aggregation, or fragmentation. In another embodiment, less than about 2% of said antibody forms an aggregate upon storage at 40° C. for at least about 4 weeks as determined by as determined by HPSEC. In another embodiment, the less than about 2% of said antibody forms an aggregate upon storage at 5° C. for at least about 3 months as determined by HPSEC. In another embodiment, less than about 2% of said antibody forms an aggregate upon storage at 5° C. for at least about 12 months as determined by HPSEC. In another embodiment, less than about 5% of said antibody is fragmented upon storage at 40° C. for at least about 4 weeks as determined by SEC. In another embodiment, less than about 5% of said antibody is fragmented upon storage at 5° C. for at least about 3 months as determined by SEC. In another embodiment, less than about 5% of said antibody is fragmented upon storage at 5° C. for at least about 12 months as determined by SEC. In another embodiment, the formulation is an injectable formulation. In another embodiment, the formulation is suitable for intravenous, subcutaneous, or intramuscular administration. In another embodiment, the formulation is suitable for aerosol administration.

Another aspect of the invention relates to a pharmaceutical unit dosage form suitable for parenteral administration to a human which comprises any of the aforementioned antibody formulations in a suitable container. In one embodiment, the antibody formulation is administered intravenously, subcutaneously, or intramuscularly.

Another aspect of the invention relates to a pharmaceutical unit dosage form suitable for aerosol administration to a human which comprises any of the aforementioned antibody formulations. In one embodiment of this aspect of the invention, the antibody formulation is administered intranasally.

Another aspect of the invention relates to a sealed container containing any of the aforementioned formulations.

Another aspect of the invention relates to a pre-filled syringe containing any of the aforementioned formulations.

Another aspect of the invention relates to a kit comprising any of the aforementioned formulations.

These and other aspects of the invention are described in further detail below.

### TERMINOLOGY

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### IL-6 and IL-6 Receptor

IL-6 is interleukin 6 IL-6 may also be referred to herein as “the antigen”.

The full length amino acid sequence of human IL-6 is SEQ ID NO: 15. This sequence is cleaved in vivo to remove an N-terminal leader peptide, to produce mature IL-6. Mature human IL-6 has amino acid sequence SEQ ID NO: 16. The mature sequence represents the in vivo circulating IL-6, which is the target antigen for therapeutic and in vivo diagnostic applications as described herein. Accordingly, IL-6 referred to herein is normally mature human IL-6, unless otherwise indicated by context.

IL-6 may be conjugated to a detectable label, such as HIS FLAG, e.g. for use in assays as described herein. For example, a fusion protein comprising IL-6 conjugated to a HIS FLAG sequence may be used.

IL-6 receptor a, IL-6Ra, is the receptor for interleukin 6 IL-6Ra is also known as IL-6Rα, IL-6Ra, IL-6R and CD126. IL-6Ra exists in vivo in a transmembrane form and in a soluble form. References to IL-6Ra may be transmembrane IL-6Ra and/or soluble IL-6Ra unless otherwise indicated by context.

IL-6 receptor referred to herein is normally human IL-6 receptor, unless otherwise indicated. An amino acid sequence of human soluble IL-6Ra (sIL-6Ra, sIL-6R) is SEQ ID NO: 17. An amino acid sequence of human transmembrane IL-6Ra is SEQ ID NO: 18.

IL-6 binds IL-6Ra to form a complex, IL-6:IL-6Ra. The complex may be either soluble (with sIL-6Ra) or membrane bound (with transmembrane IL-6Ra). When the IL-6Ra is the soluble form, the complex is designated IL-6:sIL-6Ra. References to IL-6:IL-6Ra may include IL-6 complexed with transmembrane IL-6Ra or with soluble IL-6Ra, unless otherwise indicated by context.

gp130

gp130 is a receptor for the IL-6:IL-6Ra complex. Cloning and characterization of gp130 is reported in Hibi et al, Cell 63:1149-1157 (1990). A sequence of human gp130 is set out in SEQ ID NO: 19.

### Binding Member

This describes one member of a pair of molecules that bind one another. The members of a binding pair may be naturally derived or wholly or partially synthetically produced. One member of the pair of molecules has an area on its surface, or a cavity, which binds to and is therefore complementary to a particular spatial and polar organization of the other member of the pair of molecules. Examples of types of binding pairs are antigen-antibody, biotin-avidin, hormone-hormone receptor, receptor-ligand, enzyme-substrate. The present invention is concerned with antigen-antibody type reactions.

A binding member normally comprises a molecule having an antigen-binding site. For example, a binding member may be an antibody molecule or a non-antibody protein that comprises an antigen-binding site.

An antigen binding site may be provided by means of arrangement of CDRs on non-antibody protein scaffolds, such as fibronectin or cytochrome B etc. (Haan & Maggos (2004) BioCentury, 12(5): A1-A6; Koide et al. (1998) Journal of Molecular Biology, 284: 1141-1151; Nygren et al. (1997) Curr. Op. Structural Biology, 7: 463-469), or by randomising or mutating amino acid residues of a loop within a protein scaffold to confer binding specificity for a desired target. Scaffolds for engineering novel binding sites in proteins have been reviewed in detail by Nygren et al. (Nygren et al. (1997) Curr. Op. Structural Biology, 7: 463-469). Protein scaffolds for antibody mimics are disclosed in WO/0034784, which is herein incorporated by reference in its entirety, in which the inventors describe proteins (antibody mimics) that include a fibronectin type III domain having at least one randomised loop. A suitable scaffold into which to graft one or more CDRs, e.g. a set of HCDRs, may be provided by any domain member of the immunoglobulin gene superfamily. The scaffold may be a human or non-human protein. An advantage of a non-antibody protein scaffold is that it may provide an antigen-binding site in a scaffold molecule that is smaller and/or easier to manufacture than at least some antibody molecules. Small size of a binding member may confer useful physiological properties, such as an ability to enter cells, penetrate deep into tissues or reach targets within other structures, or to bind within protein cavities of the target antigen. Use of antigen binding sites in non-antibody protein scaffolds is reviewed in Wess (Wess, L. (2004) In: BioCentury, The Bernstein Report on BioBusiness, 12(42), A1-A7). Typical are proteins having a stable backbone and one or more variable loops, in which the amino acid sequence of the loop or loops is specifically or randomly mutated to create an antigen-binding site that binds the target antigen. Such proteins include the IgG-binding domains of protein A from S. aureus, transferrin, tetranectin, fibronectin (e.g. 10th fibronectin type III domain), lipocalins as well as gamma-crystalline and other Affilin™ scaffolds (Scil Proteins). Examples of other approaches include synthetic “Microbodies” based on cyclotides—small proteins having intra-molecular disulphide bonds, Microproteins (Versabodies™, Amunix) and ankyrin repeat proteins (DARPins, Molecular Partners).

In addition to antibody sequences and/or an antigen-binding site, a binding member according to the present invention may comprise other amino acids, e.g. forming a peptide or polypeptide, such as a folded domain, or to impart to the molecule another functional characteristic in addition to ability to bind antigen. Binding members of the invention may carry a detectable label, or may be conjugated to a toxin or a targeting moiety or enzyme (e.g. via a peptidyl bond or linker). For example, a binding member may comprise a catalytic site (e.g. in an enzyme domain) as well as an antigen binding site, wherein the antigen binding site binds to the antigen and thus targets the catalytic site to the antigen. The catalytic site may inhibit biological function of the antigen, e.g. by cleavage.

Although, as noted, CDRs can be carried by non-antibody scaffolds, the structure for carrying a CDR or a set of CDRs of the invention will generally be an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains may be determined by reference to Kabat, et al. (Kabat, E. A. et al, Sequences of Proteins of Immunological Interest. 4^{th} Edition. US Department of Health and Human Services. (1987)), and updates thereof. A number of academic and commercial on-line resources are available to query this database. For example, see ref. Martin, A. C. R. (Accessing the Kabat Antibody Sequence Database by Computer PROTEINS: Structure, Function and Genetics, 25 (1996), 130-133) and the associated on-line resource, currently at the world wide web address of bioinforg.uk/abs/simkab html.

By CDR region or CDR, it is intended to indicate the hypervariable regions of the heavy and light chains of the immunoglobulin as defined by Kabat et al. (Kabat, E. A. et al. (1991) Sequences of Proteins of Immunological Interest, 5th Edition. US Department of Health and Human Services, Public Service, NIH, Washington or later editions) or Chothia and Lesk (J. Mol. Biol., 196:901-917 (1987)). An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

Among the six short CDR sequences, the third CDR of the heavy chain (HCDR3) has a greater size variability (greater diversity essentially due to the mechanisms of arrangement of the genes which give rise to it). It may be as short as 2 amino acids although the longest size known is 26. CDR length may also vary according to the length that can be accommodated by the particular underlying framework. Functionally, HCDR3 plays a role in part in the determination of the specificity of the antibody (Segal et al., (1974) PNAS, 71:4298-4302; Amit et al., (1986) Science, 233:747-753; Chothia et al., (1987) J. Mol. Biol., 196:901-917; Chothia et al., (1989) Nature, 342:877-883; Caton et al., (1990) J. Immunol., 144:1965-1968; Sharon et al., (1990) PNAS, 87:4814-4817; Sharon et al., (1990) J. Immunol., 144:4863-4869; Kabat et al., (1991) J. Immunol., 147:1709-1719; Holliger & Hudson, Nature Biotechnology 23(9):1126-1136 2005).

HCDR1 may be 5 amino acids long, consisting of Kabat residues 31-35.

HCDR2 may be 17 amino acids long, consisting of Kabat residues 50-65.

HCDR3 may be 11 or 12 amino acids long, consisting of Kabat residues 95-102, optionally including Kabat residue 100D.

LCDR1 may be 11 amino acids long, consisting of Kabat residues 24-34.

LCDR2 may be 7 amino acids long, consisting of Kabat residues 50-56.

LCDR3 may be 8 or 9 amino acids long, consisting of Kabat residues 89-97, optionally including Kabat residue 95.

### Antibody Molecule

This describes an immunoglobulin whether natural or partly or wholly synthetically produced. The term also covers any polypeptide or protein comprising an antibody antigen-binding site. It must be understood here that the invention does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described later. Antibody fragments that comprise an antibody antigen-binding site include, but are not limited to, molecules such as Fab, Fab′, Fab′-SH, scFv, Fv, dAb and Fd. Various other antibody molecules including one or more antibody antigen-binding sites have been engineered, including for example Fab2, Fab3, diabodies, triabodies, tetrabodies and minibodies. Antibody molecules and methods for their construction and use are described in Holliger & Hudson (Nature Biotechnology 23(9):1126-1136 (2005)).

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

As antibodies can be modified in a number of ways, the term “antibody molecule” should be construed as covering any binding member or substance having an antibody antigen-binding site with the required specificity and/or binding to antigen. Thus, this term covers antibody fragments and derivatives, including any polypeptide comprising an antibody antigen-binding site, whether natural or wholly or partially synthetic. Chimeric molecules comprising an antibody antigen-binding site, or equivalent, fused to another polypeptide (e.g. derived from another species or belonging to another antibody class or subclass) are therefore included. Cloning and expression of chimeric antibodies are described in EP-A-0120694 and EP-A-0125023, and a large body of subsequent literature.

Further techniques available in the art of antibody engineering have made it possible to isolate human and humanized antibodies. For example, human hybridomas can be made as described by Kontermann & Dubel (Kontermann, R & Dubel, S, Antibody Engineering, Springer-Verlag New York, LLC; 2001, ISBN: 3540413545). Phage display, another established technique for generating binding members has been described in detail in many publications, such as Kontermann & Dubel (Kontermann, R & Dubel, S, Antibody Engineering, Springer-Verlag New York, LLC; 2001, ISBN: 3540413545) and WO92/01047 (discussed further below), and U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160, U.S. Pat. No. 6,521,404.

Transgenic mice, e.g. mice in which the mouse antibody genes are inactivated and functionally replaced with human antibody genes while leaving intact other components of the mouse immune system, can be used for isolating human antibodies (Mendez, M. et al. (1997) Nature Genet, 15(2): 146-156). Humanized antibodies can be produced using techniques known in the art such as those disclosed in for example WO91/09967, U.S. Pat. No. 5,585,089, EP592106, U.S. Pat. No. 565,332 and WO93/17105. Further, WO2004/006955 describes methods for humanising antibodies, based on selecting variable region framework sequences from human antibody genes by comparing canonical CDR structure types for CDR sequences of the variable region of a non-human antibody to canonical CDR structure types for corresponding CDRs from a library of human antibody sequences, e.g. germline antibody gene segments. Human antibody variable regions having similar canonical CDR structure types to the non-human CDRs form a subset of member human antibody sequences from which to select human framework sequences. The subset members may be further ranked by amino acid similarity between the human and the non-human CDR sequences. In the method of WO2004/006955, top ranking human sequences are selected to provide the framework sequences for constructing a chimeric antibody that functionally replaces human CDR sequences with the non-human CDR counterparts using the selected subset member human frameworks, thereby providing a humanized antibody of high affinity and low immunogenicity without need for comparing framework sequences between the non-human and human antibodies. Chimeric antibodies made according to the method are also disclosed.

Synthetic antibody molecules may be created by expression from genes generated by means of oligonucleotides synthesized and assembled within suitable expression vectors, for example as described by Knappik et al. (Knappik et al. (2000) J. Mol. Biol. 296, 57-86) or Krebs et al. (Krebs et al. (2001) J. Immunological Methods 254 67-84).

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., (1989) Nature 341, 544-546; McCafferty et al (1990) Nature, 348, 552-554; Holt et al (2003) Trends in Biotechnology 21, 484-490), which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, (1988) Science, 242, 423-426; Huston et al, (1988) PNAS USA, 85, 5879-5883); (viii) bispecific single chain Fv dimers (PCT/US92/09965) and (ix) “diabodies”, multivalent or multispecific fragments constructed by gene fusion (WO94/13804; Holliger, P. et al, (1993) PNAS USA 90 6444-6448). Fv, scFv or diabody molecules may be stabilized by the incorporation of disulphide bridges linking the VH and VL domains (Reiter, Y. et al, (1996) Nature Biotech, 14, 1239-1245). Minibodies comprising a scFv joined to a CH3 domain may also be made (Hu, S. et al, (1996) Cancer Res., 56, 3055-3061). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

Qui et al. (Qui et al., (2007) Nat. Biotechnol. 25:921-929) described antibody molecules containing just two CDRs linked by a framework region. CDR3 from the VH or VL domain was linked to the CDR1 or CDR2 loop of the other domain. Linkage was through the C terminus of the selected CDR1 or CDR2 to the N terminus of the CDR3, via a FR region. Qui et al. selected the FR region having the fewest hydrophobic patches. The best combination for the antibody tested was found to be VL CDR1 linked by VH FR2 to VH CDR3 (VHCDR1-VHFR2-VLCDR3). At a molecular weight of around 3 kDa, these antibody molecules offer advantages in terms of improved tissue penetration as compared with full immunoglobulins (approx. 150 kDa) or scFv (approx. 28 kDa).

Antibody fragments of the invention can be obtained starting from a parent antibody molecule or any of the antibody molecules 2, 3, 4, 5, 7, 8, 10, 14, 16, 17, 18, 19, 21, 22 and 23, by methods such as digestion by enzymes e.g. pepsin or papain and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers, such as those supplied by the company Applied Biosystems, etc., or by nucleic acid synthesis and expression.

Functional antibody fragments according to the present invention include any functional fragment whose half-life is increased by a chemical modification, especially by PEGylation, by incorporation in a liposome by fusion to albumin or a fragment thereof.

A dAb (domain antibody) is a small monomeric antigen-binding fragment of an antibody, namely the variable region of an antibody heavy or light chain (Holt et al (2003) Trends in Biotechnology 21, 484-490). VH dAbs occur naturally in camelids (e.g. camel, llama) and may be produced by immunizing a camelid with a target antigen, isolating antigen-specific B cells and directly cloning dAb genes from individual B cells. dAbs are also producible in cell culture. Their small size, good solubility and temperature stability makes them particularly physiologically useful and suitable for selection and affinity maturation. Camelid VH dAbs are being developed for therapeutic use under the name “Nanobodies™”. A binding member of the present invention may be a dAb comprising a VH or VL domain substantially as set out herein, or a VH or VL domain comprising a set of CDRs substantially as set out herein.

Bispecific or bifunctional antibodies form a second generation of monoclonal antibodies in which two different variable regions are combined in the same molecule (Holliger and Bohlen (1999) Cancer & Metastasis Rev. 18: 411-419). Their use has been demonstrated both in the diagnostic field and in the therapy field from their capacity to recruit new effector functions or to target several molecules on the surface of tumor cells. Where bispecific antibodies are to be used, these may be conventional bispecific antibodies, which can be manufactured in a variety of ways (Holliger, P. and Winter G. (1993) Curr. Op. Biotech. 4, 446-449), e.g. prepared chemically or from hybrid hybridomas, or may be any of the bispecific antibody fragments mentioned above. These antibodies can be obtained by chemical methods (Glennie M J et al. (1987) J. Immunol 139, 2367-2375; Repp R. et al. (1995) J. Hematother. 4: 415-21) or somatic methods (Staerz U. D. and Bevan M. J. (1986) PNAS USA 83: 1453-7; Suresh M. R. et al. (1986) Method Enzymol. 121: 210-228) but likewise and preferentially by genetic engineering techniques which allow the heterodimerization to be forced and thus facilitate the process of purification of the antibody sought (Merchand et al. (1998) Nature Biotech. 16:677-681). Examples of bispecific antibodies include those of the BiTE™ technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction.

Bispecific antibodies can be constructed as entire IgG, as bispecific Fab′2, as Fab′PEG, as diabodies or else as bispecific scFv. Further, two bispecific antibodies can be linked using routine methods known in the art to form tetravalent antibodies.

Bispecific diabodies, as opposed to bispecific whole antibodies, may also be particularly useful because they can be readily constructed and expressed in E. coli. Diabodies (and many other polypeptides, such as antibody fragments) of appropriate binding specificities can be readily selected using phage display (WO94/13804) from libraries. If one arm of the diabody is to be kept constant, for instance, with a specificity directed against IL-6, then a library can be made where the other arm is varied and an antibody of appropriate specificity selected. Bispecific whole antibodies may be made by alternative engineering methods as described in Ridgeway et al., (Ridgeway, J. B. B. et al (1996) Protein Eng., 9, 616-621).

Various methods are available in the art for obtaining antibodies against IL-6. The antibodies may be monoclonal antibodies, especially of human, murine, chimeric or humanized origin, which can be obtained according to the standard methods well known to the person skilled in the art.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Köhler and Milstein (Köhler and Milstein (1975) Nature, 256:495-497).

Monoclonal antibodies can be obtained, for example, from the B cells of an animal immunized against IL-6, or one of its fragments containing the epitope recognized by said monoclonal antibodies. Suitable fragments and peptides or polypeptides comprising them are described herein, and may be used to immunise animals to generate antibodies against IL-6. Said IL-6, or one of its fragments, can especially be produced according to the usual working methods, by genetic recombination starting with a nucleic acid sequence contained in the cDNA sequence coding for IL-6 or fragment thereof, by peptide synthesis starting from a sequence of amino acids comprised in the peptide sequence of the IL-6 and/or fragment thereof.

The monoclonal antibodies can, for example, be purified on an affinity column on which IL-6 or one of its fragments containing the epitope recognized by said monoclonal antibodies, has previously been immobilized. More particularly, the monoclonal antibodies can be purified by chromatography on protein A and/or G, followed or not followed by ion-exchange chromatography aimed at eliminating the residual protein contaminants as well as the DNA and the LPS, in itself, followed or not followed by exclusion chromatography on Sepharose gel in order to eliminate the potential aggregates due to the presence of dimers or of other multimers. In one embodiment, the whole of these techniques can be used simultaneously or successively.

### Antigen-Binding Site

This describes the part of a molecule that binds to and is complementary to all or part of the target antigen. In an antibody molecule it is referred to as the antibody antigen-binding site, and comprises the part of the antibody that binds to and is complementary to all or part of the target antigen. Where an antigen is large, an antibody may only bind to a particular part of the antigen, which part is termed an epitope. An antibody antigen-binding site may be provided by one or more antibody variable domains. An antibody antigen-binding site may comprise an antibody light chain variable region (VL) and an antibody heavy chain variable region (VH).

WO2006/072620 describes engineering of antigen binding sites in structural (non-CDR) loops extending between beta strands of immunoglobulin domains. An antigen binding site may be engineered in a region of an antibody molecule separate from the natural location of the CDRs, e.g. in a framework region of a VH or VL domain, or in an antibody constant domain e g CH1 and/or CH3. An antigen binding site engineered in a structural region may be additional to, or instead of, an antigen binding site formed by sets of CDRs of a VH and VL domain. Where multiple antigen binding sites are present in an antibody molecule, they may bind the same antigen (IL-6), thereby increasing valency of the binding member. Alternatively, multiple antigen binding sites may bind different antigens (IL-6 and one or more another antigen), and this may be used to add effector functions, prolong half-life or improve in vivo delivery of the antibody molecule.

### Isolated

This refers to the state in which binding members of the invention, or nucleic acid encoding such binding members, will generally be in accordance with the present invention. Thus, binding members, VH and/or VL domains, and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes of origin other than the sequence encoding a polypeptide with the required function. Isolated members and isolated nucleic acid will be free or substantially free of material with which they are naturally associated, such as other polypeptides or nucleic acids with which they are found in their natural environment, or the environment in which they are prepared (e.g. cell culture) when such preparation is by recombinant DNA technology practised in vitro or in vivo. Members and nucleic acid may be formulated with diluents or adjuvants and still for practical purposes be isolated—for example the members will normally be mixed with gelatin or other carriers if used to coat microlitre plates for use in immunoassays, or will be mixed with pharmaceutically acceptable carriers or diluents when used in diagnosis or therapy. Binding members may be glycosylated, either naturally or by systems of heterologous eukaryotic cells (e.g. CHO or NS0 (ECACC 85110503) cells, or they may be (for example if produced by expression in a prokaryotic cell) unglycosylated.

Heterogeneous preparations comprising anti-IL-6 antibody molecules also form part of the invention. For example, such preparations may be mixtures of antibodies with full-length heavy chains and heavy chains lacking the C-terminal lysine, with various degrees of glycosylation and/or with derivatized amino acids, such as cyclisation of an N-terminal glutamic acid to form a pyroglutamic acid residue.

As used herein, the phrase “substantially as set out” refers to the characteristic(s) of the relevant CDRs of the VH or VL domain of binding members described herein will be either identical or highly similar to the specified regions of which the sequence is set out herein. As described herein, the phrase “highly similar” with respect to specified region(s) of one or more variable domains, it is contemplated that from 1 to about 5, e.g. from 1 to 4, including 1 to 3, or 1 or 2, or 3 or 4, amino acid substitutions may be made in the CDR and/or VH or VL domain.

### BRIEF DESCRIPTION OF THE FIGURES

FIG. 1 Antibody 18E, but not Antibody 18 Fc region comprises the YTE epitope. The presence of the YTE epitope in the Fc region was detected by using an anti-YTE capture antibody in an ELISA assay. The ELISA titration curves for Antibody 18 and Antibody 18E is shown.

FIG. 2 IL-6 binding by Antibody 18, Antibody 18E, IL-6 antibody A (AB A) and IL-6 antibody B (AB B) was monitored using an ELISA assays. E. coli derived recombinant IL-6 was used as capture reagent. Antibody 18 and Antibody 18E displayed substantially identical IL-6 binding activities. The EC₅₀ detected for Antibody 18 and Antibody 18E were 6.1 pM and 6.5 pM, respectively.

FIG. 3 Antibody 18 and Antibody 18E inhibit IL-6 induced TF-1 cell proliferation with substantially identical efficacy. IC₅₀ values were determined for the Antibody 18, Antibody 18E, IL-6 antibody A (AB A) and IL-6 antibody B (AB B). % maximum inhibition curves as a function of antibody concentration are shown. The IC₅₀ detected for Antibody 18 and Antibody 18E were 4.5 pM and 5.2 pM, respectively.

FIG. 4 Antibody 18 and Antibody 18E inhibit endogenous IL-6 induced VEGF release from human synovial fibroblasts with substantially identical efficacy. IC₅₀ values were determined for the Antibody 18, Antibody 18E, IL-6 antibody A (AB A) and IL-6 antibody B (AB B). % maximum inhibition curves as a function of antibody concentration are shown. The IC₅₀ detected for Antibody 18 and Antibody 18E were 1.3 pM and 1.2 pM, respectively.

FIG. 5 Pharmacokinetic profile of Antibody 18 and Antibody 18E. A single dose of 5 mg/kg of Antibody 18 or Antibody 18E was administered subcutaneously or intravenously to cynomolgous monkeys. Plasma antibody levels detected following antibody administration are shown as a function of time. The half-life of Antibody 18 is approximately 8.5 days and 9.1 days following intravenous and subcutaneous administration, respectively. The half-life of Antibody 18E is approximately 28.4 days and 28.8 days following intravenous and subcutaneous administration, respectively.

FIG. 6 Pharmacokinetic and pharmacodynamic profile of the Antibody 18 and Antibody 18E. 5 mg/kg of Antibody 18 or Antibody 18E antibody was administered subcutaneously to cynomolgous monkeys. Plasma antibody levels and plasma total IL-6 levels detected following antibody administration are shown as a function of time. The symbols represent the experimental PK and PD data and the dotted lines are the PKPD model fitted simultaneously to the PK and PD data. The estimated half-life of Antibody 18 and Antibody 18E is 9.1 days and 28.8 days, respectively. The estimated clearance of Antibody 18 and Antibody 18E is 13.1 ml/day/kg and 2.8 ml/day/kg, respectively.

FIG. 7 Simulation of free IL-6 levels in RA patient plasma following subcutaneous administration of various doses of Antibody 18E. The simulation predicts that a sustained at least 90% inhibition of IL-6 mediated signaling should be achieved by subcutaneous administration of 100 mg Antibody 18E every 8 weeks or by subcutaneous administration of 50 mg Antibody 18E every 4 weeks. The subcutaneous administration of 100 mg Antibody 18E every 12 weeks is predicted not to achieve a sustained at least 90% inhibition of IL-6 mediated signaling.

FIG. 8 Simulation of free IL-6 levels in RA patient plasma following subcutaneous administration of Antibody 18 or Antibody 18E. The simulation predicts that a sustained at least 90% inhibition of IL-6 mediated signaling should be achieved by administering a single loading dose of 200 mg Antibody 18E followed by maintenance doses of 100 mg Antibody 18E given once every 8 weeks. The simulation further predicts that the administration of 500 mg Antibody 18 every 8 weeks should not achieve a sustained at least 90% inhibition of IL-6 mediated signaling.

FIG. 9 Simulation of free IL-6 levels in RA patient plasma following subcutaneous administration of various doses of the Antibody 18 or Antibody 18E. The simulation shows that a sustained at least 90% inhibition of IL-6 mediated signaling should be achieved by administering a single subcutaneous loading dose of 100 mg Antibody 18E followed by monthly subcutaneous maintenance doses of 50 mg Antibody 18E. The simulation further predicts that a sustained at least 90% inhibition of IL-6 mediated signaling should be achieved by administering bi-weekly subcutaneous doses of 100 mg Antibody 18, but not by administering monthly subcutaneous doses of 100 mg Antibody 18.

FIG. 10. Simulation of free IL-6 levels in RA patient plasma following subcutaneous administration of various doses of the Antibody 18 or Antibody 18E. The simulation predicts that a sustained at least 90% inhibition of IL-6 mediated signaling should be achieved by administering a single subcutaneous loading dose of 200 mg Antibody 18E followed by subcutaneous maintenance doses of 100 mg Antibody 18E every 4 or 8 weeks. The simulation further predicts that a sustained at least 90% inhibition of IL-6 mediated signaling cannot be achieved by administering 100 mg Antibody 18 every 4 or 8 weeks.

FIG. 11. Shows the effect of mAab406 on hypersensitivity to heat at 46° C. in the mouse FCA tail model.

FIG. 12. Shows the effect of mAab406 on hypersensitivity to mechanical pressure in the mouse FCA tail model.

FIG. 13. Shows the effect of mAab406 on hypersensitivity to heat in the mouse FCA 24 hour model.

FIG. 14. Shows does-related effects of mAb406 on hypersensitivity to heat in the mouse FCA 48 hour model.

FIG. 15. Shows dose-related effects of mAb406 on hypersensitivity to mechanical pressure in the FCA mouse 24 hour model.

FIG. 16. Shows Dose-related effects of mAb406 on hypersensitivity to mechanical pressure in the mouse FCA 48 hour model.

FIG. 17. Shows the effect of the small molecule naproxen on hypersensitivity to heat in the mouse FCA tail model at 48 hours.

FIG. 18. Shows the effect of the small molecule naproxen on hypersensitivity to mechanical pressure in the mouse FCA tail model at 48 hours.

### DETAILED DESCRIPTION OF THE INVENTION

The present invention relates to methods for generating anti-IL-6 antibodies with extended in vivo half-life. Using the methods of the invention, an anti-IL-6 parental antibody may be modified to generate an anti-IL-6 antibody with extended in vivo half-life. Any anti-IL-6 antibody that specifically binds to the human IL-6 antigen may be used for the purpose of practicing a method of the present invention. In one embodiment, anti-IL-6 antibodies disclosed in PCT Publication No. WO 2008/065378 may be modified or used for the purpose of practicing a method of the present invention. In a specific embodiment, the anti-IL-6 antibody designated in PCT Publication No. WO 2008/065378 as Antibody 18 (hereinafter Antibody 18 or Ab 18) may be modified or used for the purpose of practicing a method of the present invention.

The present invention provides anti-IL-6 antibodies with extended in vivo half-life. In one embodiment, an anti-IL-6 antibody described herein has an extended in vivo half-life longer than that of an antibody having the same variable domains and wild type constant domains. In a specific embodiment, an anti-IL-6 antibody of the invention has an extended in vivo half-life longer than that of Antibody 18.

The present invention provides anti-IL-6 antibodies with extended in vivo half-life. In one embodiment, the half-life of an anti-IL-6 antibody of the invention is the half-life measured in a mammal. In another embodiment, the half-life of an anti-IL-6 antibody of the invention is the half-life measured in a non-human primate (for example, but not limited to cynomolgus monkey or macaque). In a further embodiment, the half-life of an anti-IL-6 antibody of the invention is the half-life measured in a human subject.

In one embodiment, the half-life of an anti-IL-6 antibody of the invention is at least 2 times, at least 3 times, at least 4 times, at least 5 times, at least 10 times or at least 20 times longer than the half-life of an antibody having the same variable domains and wild type constant domains. In another embodiment, the half-life of an anti-IL-6 antibody of the invention is 2 times, 3 times, 4 times, 5 times, 10 times or 20 times longer than the half-life of an antibody having the same variable domains and wild type constant domains. In a further embodiment, the half-life of an anti-IL-6 antibody of the invention is between 2 times and 3 times, between 2 times and 5 times, between 2 times and 10 times, between 3 times and 5 times, or between 3 times and 10 times longer than the half-life of an antibody having the same variable domains and wild type constant domains.

In one embodiment, the half-life of an anti-IL-6 antibody of the invention is at least about 2 times, at least about 3 times, at least about 4 times, at least about 5 times, at least about 10 times or at least about 20 times longer than the half-life of an antibody having the same variable domains and wild type constant domains. In another embodiment, the half-life of an anti-IL-6 antibody of the invention is about 2 times, about 3 times, about 4 times, about 5 times, about 10 times or about 20 times longer than the half-life of an antibody having the same variable domains and wild type constant domains. In a further embodiment, the half-life of an anti-IL-6 antibody of the invention is between about 2 times and about 3 times, between about 2 times and about 5 times, between about 2 times and about 10 times, between about 3 times and about 5 times, or between about 3 times and about 10 times longer than the half-life of an antibody having the same variable domains and wild type constant domains.

In one embodiment, the half-life of an anti-IL-6 antibody of the invention is at least 10 days, at least 15 days, at least 20 days, at least 25 days, at least 26 days, at least 27 days, at least 28 days, at least 29 days, at least 30 days, at least 35 days, at least 40 days, at least 45 days or at least 50 days. In another embodiment, the half-life of an anti-IL-6 antibody of the invention is 10 days, 15 days, 20 days, 25 days, 28 days, 29 days, 30 days, 35 days, 40 days, 45 days or 50 days. In a further embodiment, the half-life of an anti-IL-6 antibody of the invention is between 10 days and 20 days, between 10 days and 30 days, between 10 days and 40 days, between 10 days and 50 days, between 20 days and 30 days, between 20 days and 40 days, between 20 days and 50 days, between 25 days and 30 days, between 25 days and 40 days, between 25 days and 50 days, between 30 days and 40 days, between 30 days and 50 days or between 40 days and 50 days.

In one embodiment, the half-life of an anti-IL-6 antibody of the invention is at least about 10 days, at least about 15 days, at least about 20 days, at least 25 about days, at least about 26 days, at least about 27 days, at least about 28 days, at least 29 about days, at least about 30 days, at least about 35 days, at least about 40 days, at least about 45 days or at least about 50 days. In another embodiment, the half-life of an anti-IL-6 antibody of the invention is about 10 days, about 15 days, about 20 days, about 25 days, about 28 days, about 29 days, about 30 days, about 35 days, about 40 days, about 45 days or about 50 days. In a further embodiment, the half-life of an anti-IL-6 antibody of the invention is between about 10 days and about 20 days, between about 10 days and about 30 days, between about 10 days and about 40 days, between 10 days and about 50 days, between about 20 days and about 30 days, between about 20 days and about 40 days, between about 20 days and about 50 days, between about 25 days and about 30 days, between about 25 days and about 40 days, between v25 days and about 50 days, between about 30 days and about 40 days, between about 30 days and about 50 days or between about 40 days and about 50 days.

The present invention further provides anti-IL-6 antibodies with decreased clearance rate. The term clearance as used herein is understood to reflect the volume of plasma from which the drug substance, i.e. anti-IL-6 antibody, is completely removed per unit time. In one embodiment, an anti-IL-6 antibody described herein has a decreased clearance rate compared to the clearance rate of the parental anti-IL-6 antibody. In a specific embodiment, an anti-IL-6 antibody of the invention has a decreased clearance rate compared to that of Antibody 18.

The present invention provides anti-IL-6 antibodies with decreased clearance rate. In one embodiment, clearance rate of an anti-IL-6 antibody of the invention is the clearance rate measured in a mammal. In another embodiment, clearance rate of an anti-IL-6 antibody of the invention is the clearance rate measured in a non-human primate (for example, but not limited to cynomolgus monkey or macaque). In a further embodiment, clearance rate of an anti-IL-6 antibody of the invention is the clearance rate measured in a human subject.

In one embodiment, clearance rate of an anti-IL-6 antibody of the invention is at least 2 times, at least 3 times, at least 4 times, at least 5 times, at least 10 times or at least 20 times lower than the clearance rate of an antibody having the same variable domains and wild type constant domains. In another embodiment, clearance rate of an anti-IL-6 antibody of the invention is 2 times, 3 times, 4 times, 5 times, 10 times or 20 times lower than the clearance rate of an antibody having the same variable domains and wild type constant domains. In a further embodiment, clearance rate of an anti-IL-6 antibody of the invention is between 2 times and 3 times, between 2 times and 5 times, between 2 times and 10 times, between 3 times and 5 times, or between 3 times and 10 times lower than the clearance rate of an antibody having the same variable domains and wild type constant domains.

In one embodiment, clearance rate of an anti-IL-6 antibody of the invention is at least about 2 times, at least about 3 times, at least about 4 times, at least about 5 times, at least about 10 times or at least about 20 times lower than the clearance rate of an antibody having the same variable domains and wild type constant domains. In another embodiment, clearance rate of an anti-IL-6 antibody of the invention is about 2 times, about 3 times, about 4 times, about 5 times, about 10 times or about 20 times lower than the clearance rate of an antibody having the same variable domains and wild type constant domains. In a further embodiment, clearance rate of an anti-IL-6 antibody of the invention is between about 2 times and about 3 times, between about 2 times and about 5 times, between about 2 times and about 10 times, between about 3 times and about 5 times, or between about 3 times and about 10 times lower than the clearance rate of an antibody having the same variable domains and wild type constant domains.

In one embodiment, clearance rate of an anti-IL-6 antibody of the invention is at most 1 mL/kg/day, at most 2 mL/kg/day, at most 3 mL/kg/day, at most 4 mL/kg/day, at most 5 mL/kg/day, at most 7 mL/kg/day, at most 10 mL/kg/day, at most 15 mL/kg/day or at most 20 mL/kg/day. In another embodiment, clearance rate of an anti-IL-6 antibody of the invention is 1 mL/kg/day, 2 mL/kg/day, 3 mL/kg/day, 4 mL/kg/day, 5 mL/kg/day, 7 mL/kg/day, 10 mL/kg/day, 15 mL/kg/day or 20 mL/kg/day. In a further embodiment, clearance rate of an anti-IL-6 antibody of the invention is between 1 mL/kg/day and 2 mL/kg/day, between 1 mL/kg/day and 3 mL/kg/day, between 1 mL/kg/day and 5 mL/kg/day, between 1 mL/kg/day and 10 mL/kg/day, between 1 mL/kg/day and 15 mL/kg/day, between 2 mL/kg/day and 5 mL/kg/day, between 2 mL/kg/day and 10 mL/kg/day, between 3 mL/kg/day and 5 mL/kg/day, between 3 mL/kg/day and 10 mL/kg/day or between 5 mL/kg/day and 10 mL/kg/day.

In one embodiment, clearance rate of an anti-IL-6 antibody of the invention is at most about 1 mL/kg/day, at most about 2 mL/kg/day, at most about 3 mL/kg/day, at most about 4 mL/kg/day, at most about 5 mL/kg/day, at most about 7 mL/kg/day, at most about 10 mL/kg/day, at most about 15 mL/kg/day or at most about 20 mL/kg/day. In another embodiment, clearance rate of an anti-IL-6 antibody of the invention is about 1 mL/kg/day, about 2 mL/kg/day, about 3 mL/kg/day, about 4 mL/kg/day, about 5 mL/kg/day, about 7 mL/kg/day, about 10 mL/kg/day, about 15 mL/kg/day or about 20 mL/kg/day. In a further embodiment, clearance rate of an anti-IL-6 antibody of the invention is between about 1 mL/kg/day and about 2 mL/kg/day, between about 1 mL/kg/day and about 3 mL/kg/day, between about 1 mL/kg/day and about 5 mL/kg/day, between about 1 mL/kg/day and about 10 mL/kg/day, between about 1 mL/kg/day and about 15 mL/kg/day, between about 2 mL/kg/day and about 5 mL/kg/day, between about 2 mL/kg/day and about 10 mL/kg/day, between about 3 mL/kg/day and about 5 mL/kg/day, between about 3 mL/kg/day and about 10 mL/kg/day or between about 5 mL/kg/day and about 10 mL/kg/day.

In one embodiment, an anti-IL-6 antibody of the invention comprises a variant Fc region. In another embodiment, an anti-IL-6 antibody of the invention comprises a variant Fc region that has an altered affinity for an Fc ligand protein. In a specific embodiment, an anti-IL-6 antibody of the invention comprises a variant Fc region that has an altered affinity for FcRn. In a specific embodiment, FcRn may be a mouse, human or primate (e.g., cynomolgus) FcRn protein.

In one embodiment, an anti-IL-6 antibody of the invention comprises a variant Fc region that has an increased affinity for an Fc ligand protein. In a specific embodiment, an anti-IL-6 antibody of the invention comprises a variant Fc region that has an increased affinity for FcRn. In a specific embodiment, FcRn may be a mouse, human or primate (e.g., cynomolgus) FcRn protein.

In one embodiment, an anti-IL-6 antibody of the invention comprises a variant Fc region whose binding affinity for an Fc ligand protein is pH dependent. In a specific embodiment, an anti-IL-6 antibody of the invention comprises a variant Fc region with a pH dependent binding affinity for FcRn. In a specific embodiment, FcRn may be a mouse, human or primate (e.g., cynomolgus) FcRn protein.

In one embodiment, an anti-IL-6 antibody of the invention comprises a human IgG constant domain having one or more amino acid substitutions relative to a wild-type human IgG constant domain. In various embodiments the human IgG constant domain may be a human IgG1, IgG2, IgG3 or IgG4 constant domain. In a specific embodiment, an anti-IL-6 antibody of the invention comprises a human IgG1 constant domain having one or more amino acid substitutions relative to a wild-type human IgG1 constant domain.

In one embodiment, an anti-IL-6 antibody of the invention comprises a human IgG constant domain having one or more amino acid substitutions selected from the group consisting of: M252Y, M252F, M252W, M252T, S254T, T256S, T256R, T256Q, T256E, T256D, T256T, L309P, Q311S, H433R, H433K, H433S, H433I, H433P, H433Q, N434H, N434F, N434Y and N436H, wherein amino acid residues are numbered according to the EU index as in Kabat. In another embodiment, an anti-IL-6 antibody of the invention comprises a human IgG constant domain having one or more amino acid substitutions selected from the group consisting of: M252Y, S254T, T256E, H433K, N434F and N436H, wherein amino acid residues are numbered according to the EU index as in Kabat. In another embodiment, an anti-IL-6 antibody of the invention comprises a human IgG constant domain having one or more amino acid substitutions selected from the group consisting of: M252Y, S254T, and T256E, wherein amino acid residues are numbered according to the EU index as in Kabat. In a specific embodiment, an anti-IL-6 antibody of the invention comprises a human IgG constant domain comprising the M252Y, S254T, and T256E amino acid substitutions, wherein amino acid residues are numbered according to the EU index as in Kabat. In various embodiments the human IgG constant domain may be a human IgG1, IgG2, IgG3 or IgG4 constant domain. In a specific embodiment, an anti-IL-6 antibody of the invention comprises a human IgG1 constant domain comprising the M252Y, S254T, and T256E amino acid substitutions, wherein amino acid residues are numbered according to the EU index as in Kabat.

In one embodiment, anti-IL-6 antibodies of the invention comprise one, two, three, four, five, or all six of the CDRs of Antibody 18 (see, PCT Publication No. WO 2008/065378).

The amino acid sequences for CDR1, CDR2, and CDR3 of the heavy chain variable region of Antibody 18 defined according to Kabat are identified as SEQ ID NO:1, SEQ ID NO:2, and SEQ ID NO:3, respectively. The amino acid sequences for CDR1, CDR2 and CDR3 of the light chain variable region of Antibody 18 defined according to Kabat are identified as SEQ ID NO:4, SEQ ID NO:5, and SEQ ID NO:6, respectively.

Kabat numbering is based on the seminal work of Kabat et al. (1991) Sequences of Proteins of Immunological Interest, Publication No. 91-3242, published as a three volume set by the National Institutes of Health, National Technical Information Service (hereinafter “Kabat”). Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (latest downloadable version 1997). Any immunoglobulin sequence can be numbered according to Kabat by performing an alignment with the Kabat reference sequence. Accordingly, the Kabat numbering system provides a uniform system for numbering immunoglobulin chains. Unless indicated otherwise, all immunoglobulin amino acid sequences described herein are numbered according to the Kabat numbering system. Similarly, all single amino acid positions referred to herein are numbered according to the Kabat numbering system.

In certain embodiments, an anti-IL-6 antibody described herein may comprise a heavy chain variable region, VH, comprising at least one CDR having the amino acid sequence selected from the group consisting of SEQ ID NO:1, SEQ ID NO:2, and SEQ ID NO:3. In certain embodiments, an anti-IL-6 antibody of the invention may comprise a VH domain having the amino acid sequence of SEQ ID NO:7.

In certain embodiments, an anti-IL-6 antibody described herein may comprise a light chain variable region, VL, comprising at least one CDR having an amino acid sequence selected from the group consisting of SEQ ID NO:4, SEQ ID NO:5, and SEQ ID NO:6. In certain embodiments, an anti-IL-6 antibody of the invention may comprise a VL domain having the amino acid sequence of SEQ ID NO:8.

In one embodiment, an anti-IL-6 antibody of the invention comprises a VL domain having the amino acid sequence of SEQ ID NO:8 and further comprises a VH domain having the amino acid sequence of SEQ ID NO:7.

The present invention encompasses antibodies that bind to human IL-6, comprising derivatives of the VH domain, VH CDR1, VH CDR2, VH CDR3, VL domain, VL CDR1, VL CDR2, or VL CDR3 described herein that may bind to human IL-6. Standard techniques known to those of skill in the art can be used to introduce mutations (e.g., additions, deletions, and/or substitutions) in the nucleotide sequence encoding an antibody, including, for example, site directed mutagenesis and PCR mediated mutagenesis that are routinely used to generate amino acid substitutions. In one embodiment, the VH and/or VL CDR derivatives may include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, less than 2 amino acid substitutions, or 1 amino acid substitution relative to the original VH and/or VL CDRs of the Antibody 18 anti-IL-6 antibody. In another embodiment, the VH and/or VL CDR derivatives may have conservative amino acid substitutions (e.g. supra) made at one or more predicted non essential amino acid residues (i.e., amino acid residues which are not critical for the antibody to specifically bind to human IL-6). Mutations can also be introduced randomly along all or part of the VH and/or VL CDR coding sequences, such as by saturation mutagenesis, and the resultant mutants can be screened for biological activity to identify mutants that retain activity. Following mutagenesis, the encoded antibody can be expressed and the activity of the antibody can be determined.

The present invention further encompasses antibodies that bind to human IL-6, said antibodies or antibody fragments comprising one or more CDRs wherein said CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of Antibody 18. The percent identity of two amino acid sequences can be determined by any method known to one skilled in the art, including, but not limited to, BLAST protein searches.

The present invention further encompasses antibodies that bind to human IL-6, said antibodies or antibody fragments comprising a VH and/or a VL domain wherein said VH and/or VL domains comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of Antibody 18. The percent identity of two amino acid sequences can be determined by any method known to one skilled in the art, including, but not limited to, BLAST protein searches.

In one embodiment, an anti-IL-6 antibody of the invention may bind to human IL-6 with an affinity comparable to that of Antibody 18.

In one embodiment, an anti-IL-6 antibody of the invention specifically binds the same epitope of IL-6 as Antibody 18.

In one embodiment, an anti-IL-6 antibody specifically competes with Antibody 18 for IL-6 binding. The competition assay may be performed using any binding assay known in the art, for example, but not limited to ELISA assay or radioimmunoassay.

The invention further provides polynucleotides comprising a nucleotide sequence encoding an anti-IL-6 antibody with extended in vivo half-life. The invention also encompasses polynucleotides that hybridize under stringent or lower stringency hybridization conditions, as defined herein, to polynucleotides that encode an anti-IL-6 antibody with extended in vivo half-life.

In one embodiment, a polynucleotide of the invention encoding an anti-IL 6 antibody with extended in vivo half-life described herein comprises an optimized polynucleotide sequence. In a specific embodiment, a polynucleotide of the invention encoding the VH domain of an anti-IL-6 antibody described herein comprises the nucleotide sequence of SEQ ID NO:11. In a specific embodiment, a polynucleotide of the invention encoding the VL domain of an anti-IL-6 antibody described herein comprises the nucleotide sequence of SEQ ID NO: 12. In a specific embodiment, a polynucleotide of the invention encoding the heavy chain of an anti-IL-6 antibody described herein comprises the nucleotide sequence of SEQ ID NO: 13. In a specific embodiment, a polynucleotide of the invention encoding the light chain of an anti-IL-6 antibody described herein comprises the nucleotide sequence of SEQ ID NO: 14.

Another embodiment of the invention is a vector comprising one or more nucleotide sequences encoding an anti-IL-6 antibody with extended in vivo half-life.

In one embodiment, a vector of the invention comprises one or more nucleotide sequences encoding an anti-IL-6 antibody with extended in vivo half-life wherein the nucleotide sequence is an optimized nucleotide sequence. In a specific embodiment, a vector of the invention comprises any one of the nucleotide sequences of SEQ ID NO:11-14. In a further specific embodiment, a vector of the invention comprises one or more nucleotide sequences encoding an anti-IL-6 antibody with extended in vivo half-life wherein the nucleotide sequence is selected from the group comprising SEQ ID NO:11-14.

The present invention further relates to an isolated cell comprising a vector wherein said vector comprises one or more nucleotide sequences encoding an anti-IL-6 antibody with extended in vivo half-life. In a specific embodiment, an isolated cell of the invention comprises a polynucleotide comprising the nucleotide sequence selected from the group consisting of SEQ ID NO:11-14.

Anti-IL-6 antibodies of the invention include those of the IgG1, IgG2, IgG3, or IgG4 human isotype.

The present invention further relates to pharmaceutical compositions comprising an anti-IL-6 antibody comprising any one of the amino acid sequences of SEQ ID NO:1-10.

Anti-IL-6 antibodies described herein may have a high binding affinity for the human IL-6 antigen. For example, an antibody described herein may have an association rate constant or kₒₙ rate (antibody (Ab)+antigen (Ag)kₒₙ→Ab-Ag) of at least 2×10⁵M⁻¹ s⁻¹, at least 5×10⁵M⁻¹ s⁻¹, at least 10⁶M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹.

In another embodiment, an anti-IL-6 antibody may have a k_{off} rate ((Ab-Ag)k_{off}→antibody (Ab)+antigen (Ag)) of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, less than 5×10⁻⁴ s⁻¹, or less than 10⁻⁴ s⁻¹. In a another embodiment, an antibody of the invention has a k_{off} of less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s 1, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁸ M⁻¹ s⁻¹.

In another embodiment, an anti-IL-6 antibody may have an affinity constant or Ka (kₒₙ/k_{off}) of at least 10²M⁻¹, at least 5×10²M⁻¹, at least 10³ M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³M⁻¹, at least 5×10¹³ M 1, at least 10¹⁴M⁻¹, at least 5×10¹⁴ M⁻¹, at least 10¹⁵ M⁻¹, or at least 5×10¹⁵ M⁻¹. In yet another embodiment, an anti-IL-6 antibody may have a dissociation constant or Kd (k_{off}/kₒₙ) of less than 5×10⁻²M, less than 10⁻²M, less than 5×10⁻³M, less than 10⁻³ M, less than 5×10⁻⁴M, less than 10⁻⁴M, less than 5×10⁻⁵ M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5×10⁻¹⁰, less than 10⁻¹⁰ M, less than 5×10⁻¹¹ M, M, less than 10⁻¹¹ M, less than 5×10⁻¹² M less than 10⁻¹² M, less than 5×10⁻¹³M, less than 10⁻¹² M, less than 5×10¹⁴ M, less than 10⁻¹⁴M, less than 5×10⁻¹⁵5 M, or less than 10⁻¹⁵ M.

An antibody used in accordance with a method described herein may immunospecifically bind to IL-6 and may have a dissociation constant (Kd) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using a method described herein or known to one of skill in the art (e.g., a BIAcore assay, ELISA) (Biacore International AB, Uppsala, Sweden). In a specific embodiment, an antibody used in accordance with a method described herein may immunospecifically bind to a human IL-6 antigen and may have a dissociation constant (Kd) of between 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using a method described herein or known to one of skill in the art (e.g., a BIAcore assay, ELISA). In another embodiment, an anti-IL-6 antibody used in accordance with a method described herein may immunospecifically bind to IL-6 and may have a dissociation constant (Kd) of 500 pM, 100 pM, 75 pM or 50 pM as assessed using a method described herein or known to one of skill in the art (e.g., a BIAcore assay, ELISA).

The invention further provides polynucleotides comprising a nucleotide sequence encoding an anti-IL-6 antibody with extended in vivo half-life. The invention also encompasses polynucleotides that hybridize under stringent or lower stringency hybridization conditions, e.g., as defined herein, to polynucleotides that encode an anti-IL-6 antibody with extended in vivo half-life.

Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

A polynucleotide encoding an antibody may also be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably polyA+RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

IL-6 has been implicated in a number of disease and conditions. This disease and conditions include but are not limited to inflammation, pain and cancer. The inventive anti-IL-6 antibodies described herein, are preferably able to for example, neutralize IL-6, reduce of IL-6 levels in the body and antagonize IL-6 signalling. As such, the inventive anti-IL-6 antibodies are preferably able to act as drugs to treat these conditions and diseases.

The present invention further provides for antibodies that efficiently neutralize IL-6 activity in a subject for extended periods of time. Without being bound by a specific mechanism of action, an anti-IL-6 antibody of the invention may neutralize IL-6 by binding it and thereby preventing IL-6 from participating in protein interactions that are necessary for IL-6 mediated signal transduction. In one embodiment, an antibody of the invention is capable of reducing the plasma concentration of free (i.e. not bound by anti-IL-6 antibody) IL-6. Free IL-6 levels in a biological fluid (e.g., plasma) may be determined using quantitative bioassays, for example, but not limited to bioassays described in Papadopoulos et. al, Journal of Clinical Laboratory Analysis 9:234-37 (1995). Briefly, the bioassay measures the IL-6 induced proliferation of particular hybridoma cells (e.g., B9 hybridoma cells). The concentration of free IL-6 may also be determined by a sandwich immunoassay. Briefly, free IL-6 in serum is captured by an anti-IL-6 capture antibody. This capture antibody only binds to IL-6 in the absence of Antibody 18E and soluble IL-6 receptor. The captured IL-6 is detected by a detection antibody which does not compete with the capture antibody and is labelled with either ruthenium or HRP. The electrochemiluminescence or colorimetric signal measured is proportional to the concentration of free IL-6 in the serum. The free IL-6 concentration in serum is calculated based on a standard curve.

In one embodiment, an antibody of the invention is capable of reducing the serum concentration of free (i.e. not bound by anti-IL-6 antibody) IL-6. The administration of an effective dose of an anti-IL-6 antibody of the invention may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction in the serum concentration of free IL-6. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. The reduction in free IL-6 levels may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate.

In another embodiment, the administration of more than one dose of an anti-IL-6 antibody of the invention may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% reduction in the serum concentration of free IL-6. In one embodiment, each of the more than one dose comprises the same amount of anti-IL-6 antibody. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. In a specific embodiment, the administration of a 50 mg dose of an anti-IL-6 antibody of the invention every 4 weeks achieves a sustained at least 90% reduction in the serum concentration of free IL-6. In a specific embodiment, the administration of a 100 mg dose of an anti-IL-6 antibody of the invention every 8 weeks achieves a sustained at least 90% reduction in the serum concentration of free IL-6. In a specific embodiment, the administration of a 200 mg dose of an anti-IL-6 antibody of the invention every 12 weeks achieves a sustained at least 90% reduction in the serum concentration of free IL-6. Anti-IL-6 antibody may be administered by any method known in the art, for example but not limited to, via subcutaneous or intravenous injection. A subject may be a human or a non-human primate.

In one embodiment, an antibody of the invention is capable of neutralizing serum IL-6 in a subject. The administration of an effective dose of an anti-IL-6 antibody of the invention may achieve at least about 20%, at least about 30%, at least about 40%, at least about 50%, at least about 60%, at least about 70%, at least about 80%, at least about 90%, at least about 95%, at least about 97%, at least about 99%, or at least about 100% neutralization of serum IL-6. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. Serum IL-6 neutralization may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate.

In another embodiment, the administration of more than one dose of an anti-IL-6 antibody of the invention may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% neutralization of serum IL-6. In one embodiment, each of the more than one dose comprises the same amount of anti-IL-6 antibody. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. In a specific embodiment, the administration of a 50 mg dose of an anti-IL-6 antibody of the invention every 4 weeks achieves a sustained at least 90% neutralization of serum IL-6. In a specific embodiment, the administration of a 100 mg dose of an anti-IL-6 antibody of the invention every 8 weeks achieves a sustained at least 90% neutralization of serum IL-6. In a specific embodiment, the administration of a 200 mg dose of an anti-IL-6 antibody of the invention every 12 weeks achieves a sustained at least 90% neutralization of serum IL-6. Anti-IL-6 antibody may be administered by any method known in the art, for example but not limited to, via subcutaneous or intravenous injection. A subject may be a human or a non-human primate.

In one embodiment, an antibody of the invention is capable of inhibiting IL-6 mediated signalling in a subject. The administration of an effective dose of an anti-IL-6 antibody of the invention may achieve at least about 20%, at least about 30%, at least about 40%, at least about 50%, at least about 60%, at least about 70%, at least about 80%, at least about 90%, at least about 95%, at least about 97%, at least about 99%, or at least about 100% inhibition of IL-6 mediated signalling in a subject. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. Inhibition of IL-6 mediated signalling in a subject may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate.

In another embodiment, the administration of more than one dose of an anti-IL-6 antibody of the invention may achieve a sustained, at least about 20%, at least about 30%, at least about 40%, at least about 50%, at least about 60%, at least about 70%, at least about 80%, at least about 90%, at least about 95%, at least about 97%, at least about 99%, or at least about 100%, inhibition of IL-6 mediated signalling in a subject. In one embodiment, each of the more than one dose comprises the same amount of anti-IL-6 antibody. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. In a specific embodiment, the administration of a 50 mg dose of an anti-IL-6 antibody of the invention every 4 weeks achieves a sustained at least 90% inhibition of IL-6 mediated signalling in a subject. In a specific embodiment, the administration of a 100 mg dose of an anti-IL-6 antibody of the invention every 8 weeks achieves a sustained at least 90% inhibition of IL-6 mediated signalling in a subject. In a specific embodiment, the administration of a 200 mg dose of an anti-IL-6 antibody of the invention every 12 weeks achieves a sustained at least 90% inhibition of IL-6 mediated signalling in a subject. Anti-IL-6 antibody may be administered by any method known in the art, for example but not limited to, via subcutaneous or intravenous injection. A subject may be a human or a non-human primate.

In one embodiment, an antibody of the invention is capable of reducing synovial cell growth in a subject. The administration of an effective dose of an anti-IL-6 antibody of the invention may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction of synovial cell growth in a subject. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. Reduction of synovial cell growth in a subject may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate.

In another embodiment, the administration of more than one dose of an anti-IL-6 antibody of the invention may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% reduction of synovial cell growth in a subject. In one embodiment, each of the more than one dose comprises the same amount of anti-IL-6 antibody. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. In a specific embodiment, the administration of a 50 mg dose of an anti-IL-6 antibody of the invention every 4 weeks achieves a sustained at least 90% reduction of synovial cell growth in a subject. In a specific embodiment, the administration of a 100 mg dose of an anti-IL-6 antibody of the invention every 8 weeks achieves a sustained at least 90% reduction of synovial cell growth in a subject. In a specific embodiment, the administration of a 200 mg dose of an anti-IL-6 antibody of the invention every 12 weeks achieves a sustained at least 90% reduction of synovial cell growth in a subject. Anti-IL-6 antibody may be administered by any method known in the art, for example but not limited to, via subcutaneous or intravenous injection. A subject may be a human or a non-human primate.

In one embodiment, an antibody of the invention is capable of reducing synovial inflammation in a subject. The administration of an effective dose of an anti-IL-6 antibody of the invention may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction of synovial inflammation in a subject. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. Reduction of synovial inflammation in a subject may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate.

In another embodiment, the administration of more than one dose of an anti-IL-6 antibody of the invention may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% reduction of synovial inflammation in a subject. In one embodiment, each of the more than one dose comprises the same amount of anti-IL-6 antibody. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. In a specific embodiment, the administration of a 50 mg dose of an anti-IL-6 antibody of the invention every 4 weeks achieves a sustained at least 90% reduction of synovial inflammation in a subject. In a specific embodiment, the administration of a 100 mg dose of an anti-IL-6 antibody of the invention every 8 weeks achieves a sustained at least 90% reduction of synovial inflammation in a subject. In a specific embodiment, the administration of a 200 mg dose of an anti-IL-6 antibody of the invention every 12 weeks achieves a sustained at least 90% reduction of synovial inflammation in a subject. Anti-IL-6 antibody may be administered by any method known in the art, for example but not limited to, via subcutaneous or intravenous injection. A subject may be a human or a non-human primate.

The present invention further provides methods to reduce serum concentration of free IL-6, to neutralize serum IL-6 in a subject, to neutralize IL-6 in a subject, to inhibit IL-6 mediated signalling in a subject, to reduce synovial cell growth in a subject, and to reduce synovial inflammation in a subject.

In one embodiment, a method of reducing the serum concentration of free IL-6 (i.e. not bound by anti-IL-6 antibody) in a subject comprises administering an effective dose of an anti-IL-6 antibody with extended half-life. The administration of an effective dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction in the serum concentration of free IL-6. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. The reduction in free IL-6 levels may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate. In a specific embodiment, a method of reducing the serum concentration of free IL-6 by at least about 90% in a subject comprises administering an effective dose of 1 mg, 5 mg, 10 mg, 25 mg, 50 mg, 75 mg, 100 mg, 150 mg, 200 mg, 250 mg, 300 mg, 400 mg or 500 mg anti-IL-6 antibody with extended half-life, wherein the at least 90% reduction in the serum concentration of free IL-6 lasts for at least about 1 day, at least about 2 days, at least about 3 days, at least about 4 days, at least about 5 days, at least about 6 days, at least about 7 days, at least about 10 days, at least about 15 days, or at least about 20 days.

In one embodiment, a method of reducing the serum concentration of free IL-6 in a subject comprises administering more than one dose of an anti-IL-6 antibody with extended half-life. The administration of more than one dose of an anti-IL-6 antibody may reduce the serum concentration of free IL-6 by at least about 20%, at least about 30%, at least about 40%, at least about 50%, at least about 60%, at least about 70%, at least about 80%, at least about 90%, at least about 95%, at least about 97%, at least about 99%, or at least about 100%. In one embodiment, a method of maintaining a reduced serum concentration of free IL-6 in a subject comprises administering more than one dose of an anti-IL-6 antibody with extended half-life. The administration of more than one dose of an anti-IL-6 antibody may maintain an at least about 20%, at least about 30%, at least about 40%, at least about 50%, at least about 60%, at least about 70%, at least about 80%, at least about 90%, at least about 95%, at least about 97%, at least about 99%, or at least about 100% reduction in the serum concentration of free IL-6. In one embodiment, a method of achieving a sustained reduction in the serum concentration of free IL-6 in a subject comprises administering more than one dose of an anti-IL-6 antibody with extended half-life. The administration of more than one dose of an anti-IL-6 antibody may achieve an at least about 20%, at least about 30%, at least about 40%, at least about 50%, at least about 60%, at least about 70%, at least about 80%, at least about 90%, at least about 95%, at least about 97%, at least about 99%, or at least about 100% sustained reduction in the serum concentration of free IL-6. In one embodiment, each of the more than one dose comprises the same amount of anti-IL-6 antibody. A single dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. Anti-IL-6 antibody may be administered by any method known in the art, for example but not limited to, via subcutaneous or intravenous injection. A subject may be a human or a non-human primate.

In a specific embodiment, a method of reducing the serum concentration of free IL-6 in a subject by at least 90% comprises administering a 50 mg dose of an anti-IL-6 antibody every 4 weeks. In a specific embodiment, a method of reducing the serum concentration of free IL-6 in a subject by at least 90% comprises administering a 100 mg dose of an anti-IL-6 antibody every 8 weeks. In a specific embodiment, a method of reducing the serum concentration of free IL-6 in a subject by at least 90% comprises administering a 200 mg dose of an anti-IL-6 antibody every 12 weeks. In a specific embodiment, a method of maintaining an at least 90% reduced serum concentration of free IL-6 in a subject comprises administering a 50 mg dose of an anti-IL-6 antibody every 4 weeks. In a specific embodiment, a method of maintaining an at least 90% reduced serum concentration of free IL-6 in a subject comprises administering a 100 mg dose of an anti-IL-6 antibody every 8 weeks. In a specific embodiment, a method of maintaining an at least 90% reduced serum concentration of free IL-6 in a subject comprises administering a 200 mg dose of an anti-IL-6 antibody every 12 weeks. In a specific embodiment, a method of achieving a sustained at least 90% reduction in the serum concentration of free IL-6 in a subject comprises administering a 50 mg dose of an anti-IL-6 antibody every 4 weeks. In a specific embodiment, a method of achieving a sustained at least 90% reduction in the serum concentration of free IL-6 in a subject comprises administering a 100 mg dose of an anti-IL-6 antibody every 8 weeks. In a specific embodiment, a method of achieving a sustained at least 90% reduction in the serum concentration of free IL-6 in a subject comprises administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In a specific embodiment, a method of reducing the serum concentration of free IL-6 in a subject by at least 90% comprises (a) administering a loading dose of 100 mg anti-IL-6 antibody and (b) administering a 50 mg dose of an anti-IL-6 antibody every 4 weeks. In a specific embodiment, a method of reducing the serum concentration of free IL-6 in a subject by at least 90% comprises (a) administering a loading dose of 200 mg anti-IL-6 antibody and (b) administering a 100 mg dose of an anti-IL-6 antibody every 8 weeks. In a specific embodiment, a method of reducing the serum concentration of free IL-6 in a subject by at least 90% comprises (a) administering a loading dose of 400 mg anti-IL-6 antibody and (b) administering a 200 mg dose of an anti-IL-6 antibody every 12 weeks. In a specific embodiment, a method of maintaining an at least 90% reduced serum concentration of free IL-6 in a subject comprises (a) administering a loading dose of 100 mg anti-IL-6 antibody and (b) administering a 50 mg dose of an anti-IL-6 antibody every 4 weeks. In a specific embodiment, a method of maintaining an at least 90% reduced serum concentration of free IL-6 in a subject comprises (a) administering a loading dose of 200 mg anti-IL-6 antibody and (b) administering a 100 mg dose of an anti-IL-6 antibody every 8 weeks. In a specific embodiment, a method of maintaining an at least 90% reduced serum concentration of free IL-6 in a subject comprises (a) administering a loading dose of 400 mg anti-IL-6 antibody and (b) administering a 200 mg dose of an anti-IL-6 antibody every 12 weeks. In a specific embodiment, a method of achieving a sustained at least 90% reduction in the serum concentration of free IL-6 in a subject comprises (a) administering a loading dose of 100 mg anti-IL-6 antibody and (b) administering a 50 mg dose of an anti-IL-6 antibody every 4 weeks. In a specific embodiment, a method of achieving sustained at least 90% reduction in the serum concentration of free IL-6 in a subject comprises (a) administering a loading dose of 200 mg anti-IL-6 antibody and (b) administering a 100 mg dose of an anti-IL-6 antibody every 8 weeks. In a specific embodiment, a method of achieving sustained at least 90% reduction in the serum concentration of free IL-6 in a subject comprises (a) administering a loading dose of 400 mg anti-IL-6 antibody and (b) administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In one embodiment, a method of neutralizing serum IL-6 in a subject comprises administering an effective dose of an anti-IL-6 antibody with extended half-life. The administration of an effective dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% neutralization of serum IL-6. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. The neutralization of serum IL-6 may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate. In a specific embodiment, a method of neutralizing at least about 90% of serum IL-6 in a subject comprises administering an effective dose of 1 mg, 5 mg, 10 mg, 25 mg, 50 mg, 75 mg, 100 mg, 150 mg, 200 mg, 250 mg, 300 mg, 400 mg or 500 mg anti-IL-6 antibody with extended half-life, wherein the at least 90% neutralization of serum IL-6 lasts for at least about 1 day, at least about 2 days, at least about 3 days, at least about 4 days, at least about 5 days, at least about 6 days, at least about 7 days, at least about 10 days, at least about 15 days, or at least about 20 days.

In one embodiment, a method of neutralizing serum IL-6 in a subject comprises administering more than one dose of an anti-IL-6 antibody with extended half-life. The administration of more than one dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% neutralization of serum IL-6. In one embodiment, a method of maintaining serum IL-6 neutralization in a subject comprises administering more than one dose of an anti-IL-6 antibody with extended half-life. The administration of more than one dose of an anti-IL-6 antibody may maintain an at least about 20%, at least about 30%, at least about 40%, at least about 50%, at least about 60%, at least about 70%, at least about 80%, at least about 90%, at least about 95%, at least about 97%, at least about 99%, or at least about 100% neutralization of serum IL-6. In one embodiment, a method of achieving a sustained neutralization of serum IL-6 in a subject comprises administering more than one dose of an anti-IL-6 antibody with extended half-life. The administration of more than one dose of an anti-IL-6 antibody may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% neutralization of serum IL-6. In one embodiment, each of the more than one dose comprises the same amount of anti-IL-6 antibody. A single dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. Anti-IL-6 antibody may be administered by any method known in the art, for example but not limited to, via subcutaneous or intravenous injection. A subject may be a human or a non-human primate.

In a specific embodiment, a method of neutralizing at least about 90% of serum IL-6 in a subject comprises administering a 50 mg dose of an anti-IL-6 antibody every 4 weeks. In a specific embodiment, a method of neutralizing at least about 90% of serum IL-6 in a subject comprises administering a 100 mg dose of an anti-IL-6 antibody every 8 weeks. In a specific embodiment, a method of neutralizing at least about 90% of serum IL-6 in a subject comprises administering a 200 mg dose of an anti-IL-6 antibody every 12 weeks. In a specific embodiment, a method of maintaining an at least 90% neutralization of serum IL-6 in a subject comprises administering a 50 mg dose of an anti-IL-6 antibody every 4 weeks. In a specific embodiment, a method of maintaining an at least 90% neutralization of serum IL-6 in a subject comprises administering a 100 mg dose of an anti-IL-6 antibody every 8 weeks. In a specific embodiment, a method of maintaining an at least 90% neutralization of serum IL-6 in a subject comprises administering a 200 mg dose of an anti-IL-6 antibody every 12 weeks. In a specific embodiment, a method of achieving a sustained at least 90% neutralization of serum IL-6 in a subject comprises administering a 50 mg dose of an anti-IL-6 antibody every 4 weeks. In a specific embodiment, a method of achieving a sustained at least 90% neutralization of serum IL-6 in a subject comprises administering a 100 mg dose of an anti-IL-6 antibody every 8 weeks. In a specific embodiment, a method of achieving a sustained at least 90% neutralization of serum IL-6 in a subject comprises administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In a specific embodiment, a method of neutralizing at least about 90% of serum IL-6 in a subject comprises (a) administering a loading dose of 100 mg anti-IL-6 antibody and (b) administering a 50 mg dose of an anti-IL-6 antibody every 4 weeks. In a specific embodiment, a method of neutralizing at least about 90% of serum IL-6 in a subject comprises (a) administering a loading dose of 200 mg anti-IL-6 antibody and (b) administering a 100 mg dose of an anti-IL-6 antibody every 8 weeks. In a specific embodiment, a method of neutralizing at least about 90% of serum IL-6 in a subject comprises (a) administering a loading dose of 400 mg anti-IL-6 antibody and (b) administering a 200 mg dose of an anti-IL-6 antibody every 12 weeks. In a specific embodiment, a method of maintaining an at least 90% neutralization of serum IL-6 in a subject comprises (a) administering a loading dose of 100 mg anti-IL-6 antibody and (b) administering a 50 mg dose of an anti-IL-6 antibody every 4 weeks. In a specific embodiment, a method of maintaining an at least 90% neutralization of serum IL-6 in a subject comprises (a) administering a loading dose of 200 mg anti-IL-6 antibody and (b) administering a 100 mg dose of an anti-IL-6 antibody every 8 weeks. In a specific embodiment, a method of maintaining an at least 90% neutralization of serum IL-6 in a subject comprises (a) administering a loading dose of 400 mg anti-IL-6 antibody and (b) administering a 200 mg dose of an anti-IL-6 antibody every 12 weeks. In a specific embodiment, a method of achieving a sustained at least 90% neutralization of serum IL-6 in a subject comprises (a) administering a loading dose of 100 mg anti-IL-6 antibody and (b) administering a 50 mg dose of an anti-IL-6 antibody every 4 weeks. In a specific embodiment, a method of achieving a sustained at least 90% neutralization of serum IL-6 in a subject comprises (a) administering a loading dose of 200 mg anti-IL-6 antibody and (b) administering a 100 mg dose of an anti-IL-6 antibody every 8 weeks. In a specific embodiment, a method of achieving a sustained at least 90% neutralization of serum IL-6 in a subject comprises (a) administering a loading dose of 400 mg anti-IL-6 antibody and (b) administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In one embodiment, a method of neutralizing IL-6 in a subject comprises administering an effective dose of an anti-IL-6 antibody with extended half-life. The administration of an effective dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% neutralization of IL-6. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. The neutralization of IL-6 may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate. In a specific embodiment, a method of neutralizing at least about 90% of IL-6 in a subject comprises administering an effective dose of 1 mg, 5 mg, 10 mg, 25 mg, 50 mg, 75 mg, 100 mg, 150 mg, 200 mg, 250 mg, 300 mg, 400 mg or 500 mg anti-IL-6 antibody with extended half-life, wherein the at least 90% neutralization of IL-6 lasts for at least about 1 day, at least about 2 days, at least about 3 days, at least about 4 days, at least about 5 days, at least about 6 days, at least about 7 days, at least about 10 days, at least about 15 days, or at least about 20 days.

In one embodiment, a method of neutralizing IL-6 in a subject comprises administering more than one dose of an anti-IL-6 antibody with extended half-life. The administration of more than one dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% neutralization of IL-6. In one embodiment, a method of maintaining IL-6 neutralization in a subject comprises administering more than one dose of an anti-IL-6 antibody with extended half-life. The administration of more than one dose of an anti-IL-6 antibody may maintain an at least about 20%, at least about 30%, at least about 40%, at least about 50%, at least about 60%, at least about 70%, at least about 80%, at least about 90%, at least about 95%, at least about 97%, at least about 99%, or at least about 100% neutralization of IL-6. In one embodiment, a method of achieving a sustained neutralization of IL-6 in a subject comprises administering more than one dose of an anti-IL-6 antibody with extended half-life. The administration of more than one dose of an anti-IL-6 antibody may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% neutralization of IL-6. In one embodiment, each of the more than one dose comprises the same amount of anti-IL-6 antibody. A single dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. Anti-IL-6 antibody may be administered by any method known in the art, for example but not limited to, via subcutaneous or intravenous injection. A subject may be a human or a non-human primate.

In a specific embodiment, a method of neutralizing at least about 90% of IL-6 in a subject comprises administering a 50 mg dose of an anti-IL-6 antibody every 4 weeks. In a specific embodiment, a method of neutralizing at least about 90% of IL-6 in a subject comprises administering a 100 mg dose of an anti-IL-6 antibody every 8 weeks. In a specific embodiment, a method of neutralizing at least about 90% of IL-6 in a subject comprises administering a 200 mg dose of an anti-IL-6 antibody every 12 weeks. In a specific embodiment, a method of maintaining an at least 90% neutralization of IL-6 in a subject comprises administering a 50 mg dose of an anti-IL-6 antibody every 4 weeks. In a specific embodiment, a method of maintaining an at least 90% neutralization of IL-6 in a subject comprises administering a 100 mg dose of an anti-IL-6 antibody every 8 weeks. In a specific embodiment, a method of maintaining an at least 90% neutralization of IL-6 in a subject comprises administering a 200 mg dose of an anti-IL-6 antibody every 12 weeks. In a specific embodiment, a method of achieving a sustained at least 90% neutralization of IL-6 in a subject comprises administering a 50 mg dose of an anti-IL-6 antibody every 4 weeks. In a specific embodiment, a method of achieving a sustained at least 90% neutralization of IL-6 in a subject comprises administering a 100 mg dose of an anti-IL-6 antibody every 8 weeks. In a specific embodiment, a method of achieving a sustained at least 90% neutralization of IL-6 in a subject comprises administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In a specific embodiment, a method of neutralizing at least about 90% of IL-6 in a subject comprises (a) administering a loading dose of 100 mg anti-IL-6 antibody and (b) administering a 50 mg dose of an anti-IL-6 antibody every 4 weeks. In a specific embodiment, a method of neutralizing at least about 90% of IL-6 in a subject comprises (a) administering a loading dose of 200 mg anti-IL-6 antibody and (b) administering a 100 mg dose of an anti-IL-6 antibody every 8 weeks. In a specific embodiment, a method of neutralizing at least about 90% of IL-6 in a subject comprises (a) administering a loading dose of 400 mg anti-IL-6 antibody and (b) administering a 200 mg dose of an anti-IL-6 antibody every 12 weeks. In a specific embodiment, a method of maintaining an at least 90% neutralization of IL-6 in a subject comprises (a) administering a loading dose of 100 mg anti-IL-6 antibody and (b) administering a 50 mg dose of an anti-IL-6 antibody every 4 weeks. In a specific embodiment, a method of maintaining an at least 90% neutralization of IL-6 in a subject comprises (a) administering a loading dose of 200 mg anti-IL-6 antibody and (b) administering a 100 mg dose of an anti-IL-6 antibody every 8 weeks. In a specific embodiment, a method of maintaining an at least 90% neutralization of IL-6 in a subject comprises (a) administering a loading dose of 400 mg anti-IL-6 antibody and (b) administering a 200 mg dose of an anti-IL-6 antibody every 12 weeks. In a specific embodiment, a method of achieving a sustained at least 90% neutralization of IL-6 in a subject comprises (a) administering a loading dose of 100 mg anti-IL-6 antibody and (b) administering a 50 mg dose of an anti-IL-6 antibody every 4 weeks. In a specific embodiment, a method of achieving a sustained at least 90% neutralization of IL-6 in a subject comprises (a) administering a loading dose of 200 mg anti-IL-6 antibody and (b) administering a 100 mg dose of an anti-IL-6 antibody every 8 weeks. In a specific embodiment, a method of achieving a sustained at least 90% neutralization of IL-6 in a subject comprises (a) administering a loading dose of 400 mg anti-IL-6 antibody and (b) administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In one embodiment, a method of inhibiting IL-6 mediated signalling in a subject comprises administering an effective dose of an anti-IL-6 antibody with extended half-life. The administration of an effective dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% inhibition of IL-6 mediated signalling. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. The inhibition of IL-6 mediated signalling may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate. In a specific embodiment, a method of inhibiting at least about 90% of IL-6 mediated signalling in a subject comprises administering an effective dose of 1 mg, 5 mg, 10 mg, 25 mg, 50 mg, 75 mg, 100 mg, 150 mg, 200 mg, 250 mg, 300 mg, 400 mg or 500 mg anti-IL-6 antibody with extended half-life, wherein the at least 90% inhibition of IL-6 mediated signalling lasts for at least about 1 day, at least about 2 days, at least about 3 days, at least about 4 days, at least about 5 days, at least about 6 days, at least about 7 days, at least about 10 days, at least about 15 days, or at least about 20 days.

In one embodiment, a method of inhibiting IL-6 mediated signalling in a subject comprises administering more than one dose of an anti-IL-6 antibody with extended half-life. The administration of more than one dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% inhibition of IL-6 mediated signalling. In one embodiment, a method of maintaining inhibition of IL-6 mediated signalling in a subject comprises administering more than one dose of an anti-IL-6 antibody with extended half-life. The administration of more than one dose of an anti-IL-6 antibody may maintain an at least about 20%, at least about 30%, at least about 40%, at least about 50%, at least about 60%, at least about 70%, at least about 80%, at least about 90%, at least about 95%, at least about 97%, at least about 99%, or at least about 100% inhibition of IL-6 mediated signalling. In one embodiment, a method of achieving a sustained inhibition of IL-6 mediated signalling in a subject comprises administering more than one dose of an anti-IL-6 antibody with extended half-life. The administration of more than one dose of an anti-IL-6 antibody may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% inhibition of IL-6 mediated signalling. In one embodiment, each of the more than one dose comprises the same amount of anti-IL-6 antibody. A single dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. Anti-IL-6 antibody may be administered by any method known in the art, for example but not limited to, via subcutaneous or intravenous injection. A subject may be a human or a non-human primate.

In a specific embodiment, a method of inhibiting at least about 90% of IL-6 mediated signalling in a subject comprises administering a 50 mg dose of an anti-IL-6 antibody every 4 weeks. In a specific embodiment, a method of inhibiting at least about 90% of IL-6 mediated signalling in a subject comprises administering a 100 mg dose of an anti-IL-6 antibody every 8 weeks. In a specific embodiment, a method of inhibiting at least about 90% of IL-6 mediated signalling in a subject comprises administering a 200 mg dose of an anti-IL-6 antibody every 12 weeks. In a specific embodiment, a method of maintaining an at least 90% inhibition of IL-6 mediated signalling in a subject comprises administering a 50 mg dose of an anti-IL-6 antibody every 4 weeks. In a specific embodiment, a method of maintaining an at least 90% inhibition of IL-6 mediated signalling in a subject comprises administering a 100 mg dose of an anti-IL-6 antibody every 8 weeks. In a specific embodiment, a method of maintaining an at least 90% inhibition of IL-6 mediated signalling in a subject comprises administering a 200 mg dose of an anti-IL-6 antibody every 12 weeks. In a specific embodiment, a method of achieving a sustained at least 90% inhibition of IL-6 mediated signalling in a subject comprises administering a 50 mg dose of an anti-IL-6 antibody every 4 weeks. In a specific embodiment, a method of achieving a sustained at least 90% inhibition of IL-6 mediated signalling in a subject comprises administering a 100 mg dose of an anti-IL-6 antibody every 8 weeks. In a specific embodiment, a method of achieving a sustained at least 90% inhibition of IL-6 mediated signalling in a subject comprises administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In a specific embodiment, a method of inhibiting at least about 90% of IL-6 mediated signalling in a subject comprises (a) administering a loading dose of 100 mg anti-IL-6 antibody and (b) administering a 50 mg dose of an anti-IL-6 antibody every 4 weeks. In a specific embodiment, a method of inhibiting at least about 90% of IL-6 mediated signalling in a subject comprises (a) administering a loading dose of 200 mg anti-IL-6 antibody and (b) administering a 100 mg dose of an anti-IL-6 antibody every 8 weeks. In a specific embodiment, a method of inhibiting at least about 90% of IL-6 mediated signalling in a subject comprises (a) administering a loading dose of 400 mg anti-IL-6 antibody and (b) administering a 200 mg dose of an anti-IL-6 antibody every 12 weeks. In a specific embodiment, a method of maintaining an at least 90% inhibition of IL-6 mediated signalling in a subject comprises (a) administering a loading dose of 100 mg anti-IL-6 antibody and (b) administering a 50 mg dose of an anti-IL-6 antibody every 4 weeks. In a specific embodiment, a method of maintaining an at least 90% inhibition of IL-6 mediated signalling in a subject comprises (a) administering a loading dose of 200 mg anti-IL-6 antibody and (b) administering a 100 mg dose of an anti-IL-6 antibody every 8 weeks. In a specific embodiment, a method of maintaining an at least 90% inhibition of IL-6 mediated signalling in a subject comprises (a) administering a loading dose of 400 mg anti-IL-6 antibody and (b) administering a 200 mg dose of an anti-IL-6 antibody every 12 weeks. In a specific embodiment, a method of achieving a sustained at least 90% inhibition of IL-6 mediated signalling in a subject comprises (a) administering a loading dose of 100 mg anti-IL-6 antibody and (b) administering a 50 mg dose of an anti-IL-6 antibody every 4 weeks. In a specific embodiment, a method of achieving a sustained at least 90% inhibition of IL-6 mediated signalling in a subject comprises (a) administering a loading dose of 200 mg anti-IL-6 antibody and (b) administering a 100 mg dose of an anti-IL-6 antibody every 8 weeks. In a specific embodiment, a method of achieving a sustained at least 90% inhibition of IL-6 mediated signalling in a subject comprises (a) administering a loading dose of 400 mg anti-IL-6 antibody and (b) administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In one embodiment, a method of reducing synovial cell growth in a subject comprises administering an effective dose of an anti-IL-6 antibody with extended half-life. The administration of an effective dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial cell growth. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. The reduction in synovial cell growth may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate. In a specific embodiment, a method of reducing synovial cell growth by at least about 90% in a subject comprises administering an effective dose of 1 mg, 5 mg, 10 mg, 25 mg, 50 mg, 75 mg, 100 mg, 150 mg, 200 mg, 250 mg, 300 mg, 400 mg or 500 mg anti-IL-6 antibody with extended half-life, wherein the at least 90% reduction in synovial cell growth lasts for at least about 1 day, at least about 2 days, at least about 3 days, at least about 4 days, at least about 5 days, at least about 6 days, at least about 7 days, at least about 10 days, at least about 15 days, or at least about 20 days.

In one embodiment, a method of reducing synovial cell growth in a subject comprises administering more than one dose of an anti-IL-6 antibody with extended half-life. The administration of more than one dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial cell growth. In one embodiment, a method of maintaining reduction in synovial cell growth in a subject comprises administering more than one dose of an anti-IL-6 antibody with extended half-life. The administration of more than one dose of an anti-IL-6 antibody may maintain an at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial cell growth. In one embodiment, a method of achieving a sustained reduction in synovial cell growth in a subject comprises administering more than one dose of an anti-IL-6 antibody with extended half-life. The administration of more than one dose of an anti-IL-6 antibody may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial cell growth. In one embodiment, each of the more than one dose comprises the same amount of anti-IL-6 antibody. A single dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. Anti-IL-6 antibody may be administered by any method known in the art, for example but not limited to, via subcutaneous or intravenous injection. A subject may be a human or a non-human primate.

In a specific embodiment, a method of reducing synovial cell growth by at least about 90% in a subject comprises administering a 50 mg dose of an anti-IL-6 antibody every 4 weeks. In a specific embodiment, a method of reducing synovial cell growth by at least about 90% in a subject comprises administering a 100 mg dose of an anti-IL-6 antibody every 8 weeks. In a specific embodiment, a method of reducing synovial cell growth by at least about 90% in a subject comprises administering a 200 mg dose of an anti-IL-6 antibody every 12 weeks. In a specific embodiment, a method of maintaining an at least 90% reduction in synovial cell growth in a subject comprises administering a 50 mg dose of an anti-IL-6 antibody every 4 weeks. In a specific embodiment, a method of maintaining an at least 90% reduction in synovial cell growth in a subject comprises administering a 100 mg dose of an anti-IL-6 antibody every 8 weeks. In a specific embodiment, a method of maintaining an at least 90% reduction in synovial cell growth in a subject comprises administering a 200 mg dose of an anti-IL-6 antibody every 12 weeks. In a specific embodiment, a method of achieving a sustained at least 90% reduction in synovial cell growth in a subject comprises administering a 50 mg dose of an anti-IL-6 antibody every 4 weeks. In a specific embodiment, a method of achieving a sustained at least 90% reduction in synovial cell growth in a subject comprises administering a 100 mg dose of an anti-IL-6 antibody every 8 weeks. In a specific embodiment, a method of achieving a sustained at least 90% reduction in synovial cell growth in a subject comprises administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In a specific embodiment, a method of reducing synovial cell growth by at least about 90% in a subject comprises (a) administering a loading dose of 100 mg anti-IL-6 antibody and (b) administering a 50 mg dose of an anti-IL-6 antibody every 4 weeks. In a specific embodiment, a method of reducing synovial cell growth by at least about 90% in a subject comprises (a) administering a loading dose of 200 mg anti-IL-6 antibody and (b) administering a 100 mg dose of an anti-IL-6 antibody every 8 weeks. In a specific embodiment, a method of reducing synovial cell growth by at least about 90% in a subject comprises (a) administering a loading dose of 400 mg anti-IL-6 antibody and (b) administering a 200 mg dose of an anti-IL-6 antibody every 12 weeks. In a specific embodiment, a method of maintaining an at least 90% reduction in synovial cell growth in a subject comprises (a) administering a loading dose of 100 mg anti-IL-6 antibody and (b) administering a 50 mg dose of an anti-IL-6 antibody every 4 weeks. In a specific embodiment, a method of maintaining an at least 90% reduction in synovial cell growth in a subject comprises (a) administering a loading dose of 200 mg anti-IL-6 antibody and (b) administering a 100 mg dose of an anti-IL-6 antibody every 8 weeks. In a specific embodiment, a method of maintaining an at least 90% reduction in synovial cell growth in a subject comprises (a) administering a loading dose of 400 mg anti-IL-6 antibody and (b) administering a 200 mg dose of an anti-IL-6 antibody every 12 weeks. In a specific embodiment, a method of achieving a sustained at least 90% reduction in synovial cell growth in a subject comprises (a) administering a loading dose of 100 mg anti-IL-6 antibody and (b) administering a 50 mg dose of an anti-IL-6 antibody every 4 weeks. In a specific embodiment, a method of achieving a sustained at least 90% reduction in synovial cell growth in a subject comprises (a) administering a loading dose of 200 mg anti-IL-6 antibody and (b) administering a 100 mg dose of an anti-IL-6 antibody every 8 weeks. In a specific embodiment, a method of achieving a sustained at least 90% reduction in synovial cell growth in a subject comprises (a) administering a loading dose of 400 mg anti-IL-6 antibody and (b) administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In one embodiment, a method of reducing synovial inflammation in a subject comprises administering an effective dose of an anti-IL-6 antibody with extended half-life. The administration of an effective dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial inflammation. An effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. The reduction in synovial inflammation may last for at least about 1 day, at least about 2 days, at least about 3 days, at least about 4 days, at least about 5 days, at least about 6 days, at least about 7 days, at least about 10 days, at least about 15 days, or at least about 20 days. A subject may be a human or a non-human primate. In a specific embodiment, a method of reducing synovial inflammation by at least about 90% in a subject comprises administering an effective dose of 1 mg, 5 mg, 10 mg, 25 mg, 50 mg, 75 mg, 100 mg, 150 mg, 200 mg, 250 mg, 300 mg, 400 mg or 500 mg anti-IL-6 antibody with extended half-life, wherein the at least 90% reduction in synovial inflammation lasts for at least about 1 day, at least about 2 days, at least about 3 days, at least about 4 days, at least about 5 days, at least about 6 days, at least about 7 days, at least about 10 days, at least about 15 days, or at least about 20 days.

In one embodiment, a method of reducing synovial inflammation in a subject comprises administering more than one dose of an anti-IL-6 antibody with extended half-life. The administration of more than one dose of an anti-IL-6 antibody may achieve at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial inflammation. In one embodiment, a method of maintaining reduction in synovial inflammation in a subject comprises administering more than one dose of an anti-IL-6 antibody with extended half-life. The administration of more than one dose of an anti-IL-6 antibody may maintain an at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial inflammation. In one embodiment, a method of achieving a sustained reduction in synovial inflammation in a subject comprises administering more than one dose of an anti-IL-6 antibody with extended half-life. The administration of more than one dose of an anti-IL-6 antibody may achieve a sustained at least about 20%, at least about 30%, at least about 40%, at least about 50%, at least about 60%, at least about 70%, at least about 80%, at least about 90%, at least about 95%, at least about 97%, at least about 99%, or at least about 100% reduction in synovial inflammation. In one embodiment, each of the more than one dose comprises the same amount of anti-IL-6 antibody. A single dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In another embodiment, an initial loading dose is followed by subsequent maintenance doses. The initial loading dose may comprise twice, 3 times, 4 times, 5 times, or 10 times more of the anti-IL-6 antibody than the maintenance doses. In one embodiment, the time interval separating doses is constant. Anti-IL-6 antibody doses may be administered once a week, once every two weeks, once every three weeks, once every four weeks, once every eight weeks or once every twelve weeks. Anti-IL-6 antibody may be administered by any method known in the art, for example but not limited to, via subcutaneous or intravenous injection. A subject may be a human or a non-human primate.

In a specific embodiment, a method of reducing synovial inflammation by at least about 90% in a subject comprises administering a 50 mg dose of an anti-IL-6 antibody every 4 weeks. In a specific embodiment, a method of reducing synovial inflammation by at least about 90% in a subject comprises administering a 100 mg dose of an anti-IL-6 antibody every 8 weeks. In a specific embodiment, a method of reducing synovial inflammation by at least about 90% in a subject comprises administering a 200 mg dose of an anti-IL-6 antibody every 12 weeks. In a specific embodiment, a method of maintaining an at least 90% reduction in synovial inflammation in a subject comprises administering a 50 mg dose of an anti-IL-6 antibody every 4 weeks. In a specific embodiment, a method of maintaining an at least 90% reduction in synovial inflammation in a subject comprises administering a 100 mg dose of an anti-IL-6 antibody every 8 weeks. In a specific embodiment, a method of maintaining an at least 90% reduction in synovial inflammation in a subject comprises administering a 200 mg dose of an anti-IL-6 antibody every 12 weeks.

In a specific embodiment, a method of achieving a sustained at least 90% reduction in synovial inflammation in a subject comprises administering a 50 mg dose of an anti-IL-6 antibody every 4 weeks. In a specific embodiment, a method of achieving a sustained at least 90% reduction in synovial inflammation in a subject comprises administering a 100 mg dose of an anti-IL-6 antibody every 8 weeks. In a specific embodiment, a method of achieving a sustained at least 90% reduction in synovial inflammation in a subject comprises administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

In a specific embodiment, a method of reducing synovial inflammation by at least about 90% in a subject comprises (a) administering a loading dose of 100 mg anti-IL-6 antibody and (b) administering a 50 mg dose of an anti-IL-6 antibody every 4 weeks. In a specific embodiment, a method of reducing synovial inflammation by at least about 90% in a subject comprises (a) administering a loading dose of 200 mg anti-IL-6 antibody and (b) administering a 100 mg dose of an anti-IL-6 antibody every 8 weeks. In a specific embodiment, a method of reducing synovial inflammation by at least about 90% in a subject comprises (a) administering a loading dose of 400 mg anti-IL-6 antibody and (b) administering a 200 mg dose of an anti-IL-6 antibody every 12 weeks. In a specific embodiment, a method of maintaining an at least 90% reduction in synovial inflammation in a subject comprises (a) administering a loading dose of 100 mg anti-IL-6 antibody and (b) administering a 50 mg dose of an anti-IL-6 antibody every 4 weeks. In a specific embodiment, a method of maintaining an at least 90% reduction in synovial inflammation in a subject comprises (a) administering a loading dose of 200 mg anti-IL-6 antibody and (b) administering a 100 mg dose of an anti-IL-6 antibody every 8 weeks. In a specific embodiment, a method of maintaining an at least 90% reduction in synovial inflammation in a subject comprises (a) administering a loading dose of 400 mg anti-IL-6 antibody and (b) administering a 200 mg dose of an anti-IL-6 antibody every 12 weeks. In a specific embodiment, a method of achieving a sustained at least 90% reduction in synovial inflammation in a subject comprises (a) administering a loading dose of 100 mg anti-IL-6 antibody and (b) administering a 50 mg dose of an anti-IL-6 antibody every 4 weeks. In a specific embodiment, a method of achieving a sustained at least 90% reduction in synovial inflammation in a subject comprises (a) administering a loading dose of 200 mg anti-IL-6 antibody and (b) administering a 100 mg dose of an anti-IL-6 antibody every 8 weeks. In a specific embodiment, a method of achieving a sustained at least 90% reduction in synovial inflammation in a subject comprises (a) administering a loading dose of 400 mg anti-IL-6 antibody and (b) administering a 200 mg dose of an anti-IL-6 antibody every 12 weeks. Anti-IL-6 antibody may be administered by any method known in the art, for example but not limited to, via subcutaneous or intravenous injection. A subject may be a human or a non-human primate.

Further aspects of the present invention provide for compositions containing binding members of the invention, and their use in methods of binding, inhibiting and/or neutralising IL-6, including methods of treatment of the human or animal body by therapy.

Binding members according to the invention may be used in a method of treatment or diagnosis, such as a method of treatment (which may include prophylactic treatment) of a disease or disorder in the human or animal body (e.g. in a human patient), which comprises administering to said patient an effective amount of a binding member of the invention. Conditions treatable in accordance with the present invention include any in which IL-6 plays a role, as discussed in detail elsewhere herein.

In one embodiment, a method of treating a human in need thereof comprises administering a therapeutically effective dose of an anti-IL-6 antibody with extended half-life. In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE in a human comprises administering a therapeutically effective dose of an anti-IL-6 antibody with extended half-life. In a specific embodiment, a method of treating rheumatoid arthritis in a human comprises administering a therapeutically effective dose of an anti-IL-6 antibody with extended half-life. In a specific embodiment, a method of treating inflammatory bowel disease or SLE in a human comprises administering a therapeutically effective dose of an anti-IL-6 antibody with extended half-life. In one embodiment, a therapeutically effective dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In one embodiment, a therapeutically effective dose may comprise about 0.1-5 mg/kg, about 0.1-2 mg/kg, about 0.1-1 mg/kg, about 0.3-2 mg/kg, about 0.3-1 mg/kg, about 0.5-2 mg/kg, or about 0.5-1 mg/kg anti-IL-6 antibody. In another embodiment, a therapeutically effective dose may comprises about 20-500 mg, about 20-200 mg, about 20-100 mg, about 50-500 mg, about 50-200 mg, or about 50-100 mg anti-IL-6 antibody. An anti-IL-6 antibody may be administered using any method known in the art, for example but not limited to, via subcutaneous or intravenous injection. In a specific embodiment, a method of treating rheumatoid arthritis, inflammatory bowel disease or SLE in a human comprises administering 1 mg, 5 mg, 10 mg, 25 mg, 50 mg, 75 mg, 100 mg, 150 mg, 200 mg, 250 mg, 300 mg, 400 mg or 500 mg of an anti-IL-6 antibody with extended half-life. In a specific embodiment, a method of treating rheumatoid arthritis, inflammatory bowel disease or SLE in a human comprises administering about 0.1-5 mg/kg, about 0.1-2 mg/kg, about 0.1-1 mg/kg, about 0.3-2 mg/kg, about 0.3-1 mg/kg, about 0.5-2 mg/kg, or about 0.5-1 mg/kg of an anti-IL-6 antibody with extended half-life. In a specific embodiment, a method of treating rheumatoid arthritis, inflammatory bowel disease or SLE in a human comprises administering about 20-500 mg, about 20-200 mg, about 20-100 mg, about 50-500 mg, about 50-200 mg, or about 50-100 mg anti-IL-6 antibody of an anti-IL-6 antibody with extended half-life.

In one embodiment, a method of treating a human in need thereof comprises administering more than one dose of an anti-IL-6 antibody with extended half-life. In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE in a human comprises administering more than one dose of an anti-IL-6 antibody with extended half-life. In a specific embodiment, a method of treating rheumatoid arthritis, inflammatory bowel disease or SLE in a human comprises administering more than one dose of an anti-IL-6 antibody with extended half-life. In one embodiment, each of the more than one dose comprises the same amount of anti-IL-6 antibody. In one embodiment, a single dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In one embodiment, a single dose may comprise about 0.1-5 mg/kg, about 0.1-2 mg/kg, about 0.1-1 mg/kg, about 0.3-2 mg/kg, about 0.3-1 mg/kg, about 0.5-2 mg/kg, or about 0.5-1 mg/kg anti-IL-6 antibody. In another embodiment, a single dose may comprises about 20-500 mg, about 20-200 mg, about 20-100 mg, about 50-500 mg, about 50-200 mg, or about 50-100 mg anti-IL-6 antibody. In one embodiment, each of the more than one dose comprises the same amount of anti-IL-6 antibody. In one embodiment, an initial loading dose is followed by subsequent maintenance doses. In one embodiment, an initial loading dose may comprise twice, 3 times, 4 times, 5 times, or 10 times more of the anti-IL-6 antibody than the maintenance doses. In one embodiment, a loading dose may comprise 1 mg, 5 mg, 10 mg, 25 mg, 50 mg, 75 mg, 100 mg, 150 mg, 200 mg, 250 mg, 300 mg, 400 mg or 500 mg of an anti IL-6 antibody described herein. In one embodiment, a loading dose may comprise about 0.1-5 mg/kg, about 0.1-2 mg/kg, about 0.1-1 mg/kg, about 0.3-2 mg/kg, about 0.3-1 mg/kg, about 0.5-2 mg/kg, or about 0.5-1 mg/kg anti-IL-6 antibody. In another embodiment, a loading dose may comprise about 20-500 mg, about 20-200 mg, about 20-100 mg, about 50-500 mg, about 50-200 mg, or about 50-100 mg anti-IL-6 antibody. In one embodiment, the time interval separating the administration of doses is constant. Anti-IL-6 antibody doses may be administered once a week, once every two weeks, once every three weeks, once every four weeks, once every eight weeks, once every twelve weeks, once every sixteen weeks or once every six months. Anti-IL-6 antibody may be administered using any method known in the art, for example but not limited to, via subcutaneous or intravenous injection.

In one embodiment, a method of treating a human in need thereof comprises administering a 50 mg dose of an anti-IL-6 antibody every 4 weeks. In one embodiment, a method of treating a human in need thereof comprises administering a 100 mg dose of an anti-IL-6 antibody every 8 weeks. In one embodiment, a method of treating a human in need thereof comprises administering a 200 mg dose of an anti-IL-6 antibody every 12 weeks. In one embodiment, a method of treating a human in need thereof comprises (a) administering a loading dose of 100 mg anti-IL-6 antibody and (b) administering a 50 mg dose of an anti-IL-6 antibody every 4 weeks. In one embodiment, a method of treating a human in need thereof comprises (a) administering a loading dose of 200 mg anti-IL-6 antibody and (b) administering a 100 mg dose of an anti-IL-6 antibody every 8 weeks. In one embodiment, a method of treating a human in need thereof comprises (a) administering a loading dose of 400 mg anti-IL-6 antibody and (b) administering a 200 mg dose of an anti-IL-6 antibody every 12 weeks.

In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE comprises administering a 50 mg dose of an anti-IL-6 antibody every 4 weeks. In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE comprises administering a 100 mg dose of an anti-IL-6 antibody every 8 weeks. In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE comprises administering a 200 mg dose of an anti-IL-6 antibody every 12 weeks. In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE comprises (a) administering a loading dose of 100 mg anti-IL-6 antibody and (b) administering a 50 mg dose of an anti-IL-6 antibody every 4 weeks. In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE comprises (a) administering a loading dose of 200 mg anti-IL-6 antibody and (b) administering a 100 mg dose of an anti-IL-6 antibody every 8 weeks. In one embodiment, a method of treating rheumatoid arthritis, juvenile chronic arthritis, systemic onset juvenile arthritis, seronegative spondyloarthropathies (including ankylosing spondylitis, psoriatic arthritis and Reiter's disease), psoriasis or SLE comprises (a) administering a loading dose of 400 mg anti-IL-6 antibody and (b) administering a 200 mg dose of an anti-IL-6 antibody every 12 weeks.

In one embodiment, a method of treating rheumatoid arthritis, inflammatory bowel disease or SLE in a human comprises administering a 50 mg dose of an anti-IL-6 antibody every 4 weeks. In one embodiment, a method of treating rheumatoid arthritis, inflammatory bowel disease or SLE in a human comprises administering a 100 mg dose of an anti-IL-6 antibody every 8 weeks. In one embodiment, a method of treating rheumatoid arthritis, inflammatory bowel disease or SLE in a human comprises administering a 200 mg dose of an anti-IL-6 antibody every 12 weeks. In one embodiment, a method of treating rheumatoid arthritis, inflammatory bowel disease or SLE in a human comprises (a) administering a loading dose of 100 mg anti-IL-6 antibody and (b) administering a 50 mg dose of an anti-IL-6 antibody every 4 weeks. In one embodiment, a method of treating rheumatoid arthritis, inflammatory bowel disease or SLE in a human comprises (a) administering a loading dose of 200 mg anti-IL-6 antibody and (b) administering a 100 mg dose of an anti-IL-6 antibody every 8 weeks. In one embodiment, a method of treating rheumatoid arthritis, inflammatory bowel disease or SLE in a human comprises (a) administering a loading dose of 400 mg anti-IL-6 antibody and (b) administering a 200 mg dose of an anti-IL-6 antibody every 12 weeks.

### Anti-IL-6 Antibodies

Binding members according to the invention have been shown to neutralise IL-6 with high potency. Neutralisation means inhibition of a biological activity of IL-6. Binding members of the invention may neutralise one or more activities of IL-6. The inhibited biological activity is typically IL-6 binding to one or more of its binding partners. For example, the inhibited biological activity may be binding of IL-6 to transmembrane and/or soluble IL-6Rα. This may be demonstrated in the following assays, which are described briefly here and in more detail below: The TF-1 assay shows that binding members according to the invention inhibit IL-6 binding to membrane IL-6Ra as the TF-1 cells do not appear to produce soluble IL-6Ra. As such, the binding members of the invention therefore inhibit IL-6 binding to the membrane receptor. In the synovial fibroblast assay, binding members according to the invention inhibit IL-6 binding to soluble IL-6Ra since sIL-6Ra needs to be added to this assay for it to work. The added IL-1beta induces production of endogenous IL-6 which when inhibited by a binding member of this invention prevents VEGF production.

In accordance with the invention, binding of human or non-human primate, e.g. cynomolgus, IL-6 to IL-6Rα may be inhibited, e.g. a binding member may inhibit binding of mature human IL-6 to IL-6Rα.

Inhibition in biological activity may be partial or total. Binding members may inhibit IL-6 biological activity by 100%, or at least 95%, at least 90%, at least 85%, at least 80%, at least 75%, at least 70%, at least 60%, or at least 50% of the activity in absence of the binding member.

Neutralising potency of a binding member may be determined. Potency is normally expressed as an IC₅₀ value, in nM unless otherwise stated. In functional assays, IC₅₀ is the concentration of a binding member that reduces a biological response by 50% of its maximum. In ligand-binding studies, IC₅₀ is the concentration that reduces formation of the ligand-receptor complex by 50% of the maximal specific binding level. IC₅₀ may be calculated by plotting % of maximal biological response as a function of the log of the binding member concentration, and using a software program, such as Prism (GraphPad) or Origin (Origin Labs) to fit a sigmoidal function to the data to generate IC₅₀ values. Potency may be determined or measured using one or more assays known to the skilled person and/or as described or referred to herein.

Neutralisation of IL-6 activity by a binding member in an assay described herein, e.g. the TF-1 proliferation assay or other cell-based assays described below, indicates that the binding member binds and neutralises IL-6. Other methods that may be used for determining binding of a binding member to IL-6 include ELISA, Western blotting, immunoprecipitation, affinity chromatography and biochemical assays.

Binding members described herein were demonstrated to bind and neutralise biological effects of endogenous human IL-6, as shown in an assay of inhibition of VEGF release from human synovial fibroblasts in response to endogenous human IL-6, reported in Examples 1.7 and 2.7 herein. In this assay, synovial fibroblasts from rheumatoid arthritis patients produce IL-6 in response to stimulation with IL-1β and soluble IL-6Rα, leading to IL-6 induced secretion of VEGF. The IL-6 produced by the human synovial fibroblasts thus represents endogenous human IL-6. Endogenous IL-6 is the molecular target for medical treatment in humans, so neutralisation of endogenous IL-6 is an important indicator of the therapeutic potential of the binding members. Since the assays were conducted with synovial fibroblasts obtained from rheumatoid arthritis patients, the results are particularly relevant to use of the binding members for treating rheumatoid arthritis. Neutralising potency of optimised antibody molecules tested in the VEGF release assay surpassed that of the known anti IL-6 antibody CNTO-328.

A binding member according to the invention may have an IC₅₀ of less than 50 nM, e.g. less than 5 nM, e.g. less than 1 nM in an assay of inhibition of VEGF release from human synovial fibroblasts stimulated with 0.6 pM human IL-1β and 2.4 nM soluble human IL-6Rα.

Endogenous IL-6 is known to be a mixture of glycosylated and unglycosylated forms. Binding of a binding member of the invention to endogenous IL-6 has been demonstrated in the synovial fibroblast assay since this assay utilises IL-6 from human synovial fibroblasts i.e. endogenous IL-6.

A binding member of the invention may inhibit IL-6 induced proliferation of TF-1 cells. TF-1 is a human premyeloid cell line established from a patient with erythroleukaemia (Kitamura et al 1989). The TF-1 cell line requires the presence of a growth factor for survival and proliferation. The individual growth factors TF-1 cells can respond to include IL-6, GM-CSF and Oncostatin M A binding member of the invention may have an IC₅₀ of less than 100 nM, e.g. less than 20 nM, 10 nM or 1 nM, e.g. less than 100 pM, 70 pM, 50 pM, 40 pM, 30 pM, 20 pM or 10 pM, in an assay for inhibition of proliferation of TF-1 cells in response to 20 pM human IL-6. As described herein (see Example 1.5), a parent IgG “CAN022D10” was shown to have an IC₅₀ in the TF-1 proliferation assay of about 93 nM, and we subsequently generated optimised variants of CAN022D10 having substantially increased potency (IC₅₀ generally less than 100 pM), as shown in Examples 2.2, 2.5 and 2.6 (Tables 3, 4 and 5, respectively). Notably, IC₅₀ values for some of the optimised clones were measured to be low as 5 pM or less, for example the germlined IgG Antibody 7, Antibody 17 and Antibody 18, representing extremely high neutralising potency of these antibodies.

A binding member of the invention may inhibit IL-6 induced proliferation of B9 cells. B9 cells are a sub-clone of the murine B-cell hybridoma cell line, B13.29, selected on the basis of their specific response to IL-6. B9 cells require IL-6 for survival and proliferation and respond to very low concentrations of IL-6. As such, proliferation of these cells in the presence of an IL-6 antibody can be assessed and the affinity of the antibody determined. Example 2.10 herein shows that Antibody 18 inhibited B9 cell proliferation in response to IL-6, and showed high affinity in this assay.

Auto-antibody production in rheumatoid arthritis is mostly of the IgM class. SKW6.4 is a clonal IgM secreting human lymphoblastoid B cell line. Upon stimulation with IL-6 these cells secrete IgM, thus this assay was perceived to be relevant to rheumatoid arthritis. SKW6.4 cells may be used in an assay to determine potency of binding members for neutralising IL-6, by determining inhibition of IgM secretion in response to IL-6. A binding member of the invention may have an IC₅₀ of less than 10 pM, e.g. less than 5 pM, in an SKW6.4 cell assay of inhibition of IgM secretion in response to 100 pM human IL-6. Antibody 18 was shown to neutralise effects of IL-6 in this assay—see Example 2.11 (Table 9).

The invention provides high affinity binding members for human IL-6. High affinity for IL-6 from cynomolgus monkey was also demonstrated. A binding member of the invention may bind human IL-6 and/or cynomolgus IL-6 with a KD of not more than 1 nM, e.g. not more than 100 pM, 50 pM, 30 pM or 10 pM. The KD may be determined by surface plasmon resonance, e.g. BIAcore®. BIAcore® measurements of affinity are described herein in Example 2.9. Remarkably, the affinity of Antibodies 7 and 18 was found to be beyond the limit measurable using the BIAcore® instrument, indicating a KD value below 10 pM.

As described elsewhere herein, surface plasmon resonance involves passing an analyte in fluid phase over a ligand attached to a support, and determining binding between analyte and ligand. Surface plasmon resonance may for example be performed whereby IL-6 is passed in fluid phase over a binding member attached to a support. Surface plasmon resonance data may be fitted to a monovalent analyte data model. An affinity constant Kd may be calculated from the ratio of rate constants kd/ka as determined by surface plasmon resonance using a monovalent analyte data model.

Affinity of a binding member for IL-6 may alternatively be calculated by Schild analysis, e.g. based on an assay of inhibition of TF-1 cell proliferation in response to varied concentrations of human IL-6. A binding member of the invention may have an affinity of less than 10 pM, e.g. less than 1 pM, as calculated by Schild analysis. As reported in Example 2.10 herein, the affinity of Antibody 18 for human IL-6 was calculated as 0.4 pM using Schild analysis.

A binding member of the invention may optionally not cross-react with one or more, or all, of the following: leukaemia inhibitory factor (LIF), ciliary neurotrophic factor (CNTF), IL-11 or oncostatin M.

A binding member of the invention may optionally not cross-react with rat IL-6, mouse IL-6 and/or dog IL-6.

Cross-reactivity of binding members for binding other proteins or non-human IL-6 may be tested for example in a time resolved fluorescence assay for inhibition of human IL-6 binding to the binding member immobilised on a support, such as the DELFIA® epitope competition assay as described in Example 1.6. For example, any or all of LIF, CNTF, IL-11, oncostatin M, rat IL-6 and mouse IL-6 may show no inhibition, less than 50% inhibition, or may have an IC₅₀ greater than 0.5 mM or greater than 1 mM in the time resolved fluorescence assay for inhibition of labelled human IL-6 binding to the binding member immobilised on a support. For example, any or all of LIF, CNTF, IL-11, oncostatin M, rat IL-6 and mouse IL-6 may show no inhibition or may have an IC₅₀ at least 10- or 100-fold greater than that of unlabelled human IL-6 in the time resolved fluorescence assay for testing cross-reactivity. In this assay, labelled wild type mature human IL-6 is used at a final concentration of the Kd of its interaction with the binding member.

A binding member of the invention may cross-react with cynomolgus IL-6. Cross-reactivity may be determined as inhibition of labelled human IL-6 binding to the binding member immobilised on a support, in the time resolved fluorescence assay described above. For example, cynomolgus IL-6 may have an IC₅₀ of less than 5 nM, e.g. less than 2.5 nM, e.g. about 1 nM, in this time resolved fluorescence assay. Cynomolgus IL-6 may have an IC₅₀ less than 10-fold different, e.g. less than 5-fold different, from the IC₅₀ of unlabelled human IL-6 in this assay.

In one embodiment, an anti-IL-6 antibody binds an epitope on IL-6 that is conserved between the human and cynomolgus IL-6 sequences, and is different in the mouse, rat and dog IL-6 sequence compared with the human sequence.

In one embodiment, binding members bind the “site 1” region of IL-6, which is the region that interacts with IL-6Rα. Binding members of the invention may thus competitively inhibit IL-6 binding to IL-6Rα, thereby neutralising biological effects of IL-6 that are mediated through IL-6Rα.

A binding member of the invention may bind human IL-6 at Phe 102 and/or Ser204. A binding member of the invention may also bind human IL-6 at Arg207. Optionally a binding member may bind flanking residues or structurally neighbouring residues in the IL-6 molecule, in addition to binding Phe102 and/or Ser 204. By convention, residue numbering corresponds to full length human IL-6 (SEQ ID NO: 15). However, binding may be determined using mature human IL-6. Binding to IL-6 residues is as determined by site directed mutagenesis, as explained below.

Mutagenesis of single amino acids and regions of proteins in order to correlate structure with activity is well known to one skilled in the art and has been used to define regions of proteins that bind to antibodies (Lu et al., (2005) Biochemistry 44:11106-14). Binding to and/or neutralisation of mutant human IL-6 may be used to assess whether a binding member binds Phe102, Ser204 and/or Arg207. Absence of binding or neutralisation, or significantly reduced binding or neutralisation, with mutant IL-6 compared with wild-type indicates that a binding member binds the mutated residue.

Binding to a residue in IL-6 may be determined using IL-6 mutated at the selected residue in a time resolved fluorescence assay of inhibition of labelled wild type human IL-6 binding to the binding member immobilised on a support, wherein the labelled wild type mature human IL-6 is at a final concentration equal to the Kd of its interaction with the binding member. Where the mutant IL-6 does not inhibit binding of labelled wild type IL-6 to the binding member, or where the mutant IL-6 has an IC₅₀ greater than that of unlabelled wild type IL-6 (e.g. more than 10-fold or 100-fold greater), this indicates that the mutated residue is bound by the binding member.

A binding member of the invention may optionally not bind and/or neutralise mutant human IL-6 having a mutation at residue Phe102, Ser204 and/or Arg207, e.g. mutation Phe102Glu, Ser204Glu, Ser204Tyr and/or Arg207Glu.

A binding member of the invention may comprise an antibody molecule, e g a human antibody molecule. The binding member normally comprises an antibody VH and/or VL domain VH and VL domains of binding members are also provided as part of the invention. Within each of the VH and VL domains are complementarity determining regions, (“CDRs”), and framework regions, (“FRs”). A VH domain comprises a set of HCDRs, and a VL domain comprises a set of LCDRs. An antibody molecule may comprise an antibody VH domain comprising a VH CDR1, CDR2 and CDR3 and a framework. It may alternatively or also comprise an antibody VL domain comprising a VL CDR1, CDR2 and CDR3 and a framework. A VH or VL domain framework comprises four framework regions, FR1, FR2, FR3 and FR4, interspersed with CDRs in the following structure: FR1-CDR1-FR2-CDR2-FR3-CDR3-FR4.

Examples of antibody VH and VL domains and CDRs according to the present invention are as listed in the sequence listing that forms part of the present disclosure. Further CDRs are disclosed in PCT Publication No. WO 2008/065378. All VH and VL sequences, CDR sequences, sets of CDRs and sets of HCDRs and sets of LCDRs disclosed herein and in PCT Publication No. WO 2008/065378 represent aspects and embodiments of the invention. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. Unless otherwise stated, a “set of CDRs” includes HCDRs and LCDRs. Typically binding members of the invention are monoclonal antibodies.

A binding member of the invention may comprise an antigen-binding site within a non-antibody molecule, normally provided by one or more CDRs e.g. a set of CDRs in a non-antibody protein scaffold, as discussed further below.

As noted above, a binding member in accordance with the present invention modulates and may neutralise a biological activity of IL-6. As described herein, IL-6-binding members of the present invention may be optimised for neutralizing potency. Generally, potency optimisation involves mutating the sequence of a selected binding member (normally the variable domain sequence of an antibody) to generate a library of binding members, which are then assayed for potency and the more potent binding members are selected. Thus selected “potency-optimised” binding members tend to have a higher potency than the binding member from which the library was generated. Nevertheless, high potency binding members may also be obtained without optimisation, for example a high potency binding member may be obtained directly from an initial screen e.g. a biochemical neutralization assay. A “potency optimized” binding member refers to a binding member with an optimized potency of neutralization of a particular activity or downstream function of IL-6. Assays and potencies are described in more detail elsewhere herein. The present invention provides both potency-optimized and non-optimized binding members, as well as methods for potency optimization from a selected binding member. The present invention thus allows the skilled person to generate binding members having high potency.

In a further aspect, the present invention provides a method of obtaining one or more binding members able to bind the antigen, the method including bringing into contact a library of binding members according to the invention and said antigen, and selecting one or more binding members of the library able to bind said antigen.

The library may be displayed on particles or molecular complexes, e.g. replicable genetic packages, such as yeast, bacterial or bacteriophage (e.g. T7) particles, viruses, cells or covalent, ribosomal or other in vitro display systems, each particle or molecular complex containing nucleic acid encoding the antibody VH variable domain displayed on it, and optionally also a displayed VL domain if present. Phage display is described in WO92/01047 and e.g. U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160 and U.S. Pat. No. 6,521,404, each of which is herein incorporated by reference in their entirety.

Following selection of binding members able to bind the antigen and displayed on bacteriophage or other library particles or molecular complexes, nucleic acid may be taken from a bacteriophage or other particle or molecular complex displaying a said selected binding member. Such nucleic acid may be used in subsequent production of a binding member or an antibody VH or VL variable domain by expression from nucleic acid with the sequence of nucleic acid taken from a bacteriophage or other particle or molecular complex displaying a said selected binding member.

Variants of the VH and VL domains and CDRs of the present invention, including those for which amino acid sequences are set out herein, and which can be employed in binding members of the invention can be obtained by means of methods of sequence alteration or mutation and screening for antigen binding members with desired characteristics. Examples of desired characteristics include but are not limited to:
Increased binding affinity for antigen relative to known antibodies which are specific for the antigen
Increased neutralization of an antigen activity relative to known antibodies which are specific for the antigen if the activity is known
Specified competitive ability with a known antibody or ligand to the antigen at a specific molar ratio
Ability to immunoprecipitate complex
Ability to bind to a specified epitope
   Linear epitope, e.g. peptide sequence identified using peptide-binding scan as described herein, e.g. using peptides screened in linear and/or constrained conformation
   Conformational epitope, formed by non-continuous residues
Ability to modulate a new biological activity of IL-6, or downstream molecule.
   Such methods are also provided herein.

Variants of antibody molecules disclosed herein may be produced and used in the present invention. Following the lead of computational chemistry in applying multivariate data analysis techniques to the structure/property-activity relationships (Wold, et al. Multivariate data analysis in chemistry. Chemometrics—Mathematics and Statistics in Chemistry (Ed.: B. Kowalski), D. Reidel Publishing Company, Dordrecht, Holland, 1984 (ISBN 90-277-1846-6)) quantitative activity-property relationships of antibodies can be derived using well-known mathematical techniques, such as statistical regression, pattern recognition and classification (Norman et al. Applied Regression Analysis. Wiley-Interscience; 3rd edition (April 1998) ISBN: 0471170828; Kandel, Abraham & Backer, Eric. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. & Frank, Eibe. Data Mining: Practical Machine Learning Tools and Techniques with Java Implementations. Morgan Kaufmann; (Oct. 11, 1999), ISBN: 1558605525; Denison David G. T. (Editor), Christopher C. Holmes, Bani K. Mallick, Adrian F. M. Smith. Bayesian Methods for Nonlinear Classification and Regression (Wiley Series in Probability and Statistics). John Wiley & Sons; (July 2002), ISBN: 0471490369; Ghose, Amp K. & Viswanadhan, Vellarkad N. Combinatorial Library Design and Evaluation Principles, Software, Tools, and Applications in Drug Discovery. ISBN: 0-8247-0487-8). The properties of antibodies can be derived from empirical and theoretical models (for example, analysis of likely contact residues or calculated physicochemical property) of antibody sequence, functional and three-dimensional structures and these properties can be considered singly and in combination.

An antibody antigen-binding site composed of a VH domain and a VL domain is typically formed by six loops of polypeptide: three from the light chain variable domain (VL) and three from the heavy chain variable domain (VH). Analysis of antibodies of known atomic structure has elucidated relationships between the sequence and three-dimensional structure of antibody combining sites (Chothia C. et al. (1992) J. Molecular Biology 227, 799-817; Al-Lazikani, et al. (1997) J. Molecular Biology 273(4), 927-948). These relationships imply that, except for the third region (loop) in VH domains, binding site loops have one of a small number of main-chain conformations: canonical structures. The canonical structure formed in a particular loop has been shown to be determined by its size and the presence of certain residues at key sites in both the loop and in framework regions (Chothia C. et al. (1992) J. Molecular Biology 227, 799-817; Al-Lazikani, et al. (1997) J. Molecular Biology 273(4), 927-948).

This study of sequence-structure relationship can be used for prediction of those residues in an antibody of known sequence, but of an unknown three-dimensional structure, which are important in maintaining the three-dimensional structure of its CDR loops and hence maintain binding specificity. These predictions can be backed up by comparison of the predictions to the output from lead optimization experiments. In a structural approach, a model can be created of the antibody molecule (Chothia, et al. (1986) Science, 223, 755-758) using any freely available or commercial package, such as WAM (Whitelegg, N. R. u. & Rees, A. R (2000). Prot. Eng., 12, 815-824). A protein visualisation and analysis software package, such as Insight II (Accelrys, Inc.) or Deep View (Guex, N. and Peitsch, M. C. (1997) Electrophoresis 18, 2714-2723) may then be used to evaluate possible substitutions at each position in the CDR. This information may then be used to make substitutions likely to have a minimal or beneficial effect on activity.

The techniques required to make substitutions within amino acid sequences of CDRs, antibody VH or VL domains and binding members generally are available in the art. Variant sequences may be made, with substitutions that may or may not be predicted to have a minimal or beneficial effect on activity, and tested for ability to bind and/or neutralize IL-6 and/or for any other desired property.

Variable domain amino acid sequence variants of any of the VH and VL domains whose sequences are specifically disclosed herein may be employed in accordance with the present invention, as discussed.

Variants of VL domains of the invention, and binding members or antibody molecules comprising them, include VL domains in which Arginine is not present at Kabat residue 108, e.g. where Kabat residue 108 is a different residue or is deleted. For example, an antibody molecule, such as an antibody molecule lacking a constant domain, e g an scFv, may comprise a VL domain having a VL domain sequence or variant thereof as described herein, in which Arginine at Kabat residue 108 an amino acid residue other than Arginine or is deleted.

A further aspect of the invention is an antibody molecule comprising a VH domain that has at least 60, 70, 80, 85, 90, 95, 98 or 99% amino acid sequence identity with a VH domain of Antibody 18 shown in the appended sequence listing, and/or comprising a VL domain that has at least 60, 70, 80, 85, 90, 95, 98 or 99% amino acid sequence identity with a VL domain of Antibody 18 shown in the appended sequence listing. Algorithms that can be used to calculate % identity of two amino acid sequences include e.g. BLAST (Altschul et al. (1990) J. Mol. Biol. 215: 405-410), FASTA (Pearson and Lipman (1988) PNAS USA 85: 2444-2448), or the Smith-Waterman algorithm (Smith and Waterman (1981) J. Mol Biol. 147: 195-197), e.g. employing default parameters.

Particular variants may include one or more amino acid sequence alterations (addition, deletion, substitution and/or insertion of an amino acid residue).

Alterations may be made in one or more framework regions and/or one or more CDRs. The alterations normally do not result in loss of function, so a binding member comprising a thus-altered amino acid sequence may retain an ability to bind and/or neutralize IL-6. It may retain the same quantitative binding and/or neutralizing ability as a binding member in which the alteration is not made, e.g. as measured in an assay described herein. The binding member comprising a thus-altered amino acid sequence may have an improved ability to bind and/or neutralize IL-6.

Alteration may comprise replacing one or more amino acid residue with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences of the invention are described elsewhere herein. Naturally occurring amino acids include the 20 “standard” 1-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. (Voet & Voet, Biochemistry, 2nd Edition, (Wiley) 1995). Those amino acid residues that are derivatised at their N-alpha position will only be located at the N-terminus of an amino-acid sequence. Normally in the present invention an amino acid is an 1-amino acid, but it may be a d-amino acid. Alteration may therefore comprise modifying an 1-amino acid into, or replacing it with, a d-amino acid. Methylated, acetylated and/or phosphorylated forms of amino acids are also known, and amino acids in the present invention may be subject to such modification.

Amino acid sequences in antibody domains and binding members of the invention may comprise non-natural or non-standard amino acids described above. Non-standard amino acids (e.g. d-amino acids) may be incorporated into an amino acid sequence during synthesis, or by modification or replacement of the “original” standard amino acids after synthesis of the amino acid sequence.

Use of non-standard and/or non-naturally occurring amino acids increases structural and functional diversity, and can thus increase the potential for achieving desired IL-6-binding and neutralizing properties in a binding member of the invention. Additionally, d-amino acids and analogues have been shown to have different pharmacokinetic profiles compared with standard 1-amino acids, owing to in vivo degradation of polypeptides having 1-amino acids after administration to an animal e g a human, meaning that d-amino acids are advantageous for some in vivo applications.

Novel VH or VL regions carrying CDR-derived sequences of the invention may be generated using random mutagenesis of one or more selected VH and/or VL genes to generate mutations within the entire variable domain. Such a technique is described by Gram et al. (Gram et al., (1992) PNAS USA, 89:3576-3580), who used error-prone PCR. In some embodiments one or two amino acid substitutions are made within an entire variable domain or set of CDRs.

Another method that may be used is to direct mutagenesis to CDR regions of VH or VL genes. Such techniques are disclosed by Barbas et al. (Barbas et al., (1994) PNAS USA, 91:3809-3813) and Schier et al. (Schier et al., (1996) J. Mol. Biol. 263:551-567).

All the above-described techniques are known as such in the art and the skilled person will be able to use such techniques to provide binding members of the invention using routine methodology in the art.

A further aspect of the invention provides a method for obtaining an antibody antigen-binding site for IL-6, the method comprising providing by way of addition, deletion, substitution or insertion of one or more amino acids in the amino acid sequence of a VH domain set out herein a VH domain which is an amino acid sequence variant of the VH domain, optionally combining the VH domain thus provided with one or more VL domains, and testing the VH domain or VH/VL combination or combinations to identify a binding member or an antibody antigen-binding site for IL-6 and optionally with one or more desired properties, e.g. ability to neutralize IL-6 activity. Said VL domain may have an amino acid sequence which is substantially as set out herein. An analogous method may be employed in which one or more sequence variants of a VL domain disclosed herein are combined with one or more VH domains.

As noted above, a CDR amino acid sequence substantially as set out herein may be carried as a CDR in a human antibody variable domain or a substantial portion thereof. The HCDR3 sequences substantially as set out herein represent embodiments of the present invention and each of these may be carried as a HCDR3 in a human heavy chain variable domain or a substantial portion thereof.

Variable domains employed in the invention may be obtained or derived from any germline or rearranged human variable domain, or may be a synthetic variable domain based on consensus or actual sequences of known human variable domains. A variable domain can be derived from a non-human antibody. A CDR sequence of the invention (e.g. CDR3) may be introduced into a repertoire of variable domains lacking a CDR (e.g. CDR3), using recombinant DNA technology. For example, Marks et al. (Marks et al (1992) Bio/Technology 10:779-783) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire may be combined with a CDR3 of a particular antibody. Using analogous techniques, the CDR3-derived sequences of the present invention may be shuffled with repertoires of VH or VL domains lacking a CDR3, and the shuffled complete VH or VL domains combined with a cognate VL or VH domain to provide binding members of the invention. The repertoire may then be displayed in a suitable host system, such as the phage display system of WO92/01047, which is herein incorporated by reference in its entirety, or any of a subsequent large body of literature, including Kay, Winter & McCafferty (Kay, B. K., Winter, J., and McCafferty, J. (1996) Phage Display of Peptides and Proteins: A Laboratory Manual, San Diego: Academic Press), so that suitable binding members may be selected. A repertoire may consist of from anything from 104 individual members upwards, for example at least 105, at least 106, at least 107, at least 108, at least 109 or at least 1010 members or more. Other suitable host systems include, but are not limited to yeast display, bacterial display, T7 display, viral display, cell display, ribosome display and covalent display.

A method of preparing a binding member for IL-6 antigen is provided, which method comprises:

(a) providing a starting repertoire of nucleic acids encoding a VH domain which either include a CDR3 to be replaced or lack a CDR3 encoding region;

(b) combining said repertoire with a donor nucleic acid encoding an amino acid sequence substantially as set out herein for a VH CDR3 such that said donor nucleic acid is inserted into the CDR3 region in the repertoire, so as to provide a product repertoire of nucleic acids encoding a VH domain;

(c) expressing the nucleic acids of said product repertoire;

(d) selecting a binding member for IL-6; and

(e) recovering said binding member or nucleic acid encoding it.

Again, an analogous method may be employed in which a VL CDR3 of the invention is combined with a repertoire of nucleic acids encoding a VL domain that either include a CDR3 to be replaced or lack a CDR3 encoding region.

Similarly, one or more, or all three CDRs may be grafted into a repertoire of VH or VL domains that are then screened for a binding member or binding members for IL-6.

Similarly, other VH and VL domains, sets of CDRs and sets of HCDRs and/or sets of LCDRs disclosed herein may be employed.

A substantial portion of an immunoglobulin variable domain may comprise at least the three CDR regions, together with their intervening framework regions. The portion may also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of binding members of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including antibody constant regions, other variable domains (for example in the production of diabodies) or detectable/functional labels as discussed in more detail elsewhere herein.

Although in some aspects of the invention, binding members comprise a pair of VH and VL domains, single binding domains based on either VH or VL domain sequences form further aspects of the invention. It is known that single immunoglobulin domains, especially VH domains, are capable of binding target antigens in a specific manner. For example, see the discussion of dAbs above.

In the case of either of the single binding domains, these domains may be used to screen for complementary domains capable of forming a two-domain binding member able to bind IL-6. This may be achieved by phage display screening methods using the so-called hierarchical dual combinatorial approach as disclosed in WO92/01047, herein incorporated by reference in its entirety, in which an individual colony containing either an H or L chain clone is used to infect a complete library of clones encoding the other chain (L or H) and the resulting two-chain binding member is selected in accordance with phage display techniques, such as those described in that reference. This technique is also disclosed in Marks et al, ibid. (Marks et al (1992) Bio/Technology 10:779-783).

Binding members of the present invention may further comprise antibody constant regions or parts thereof, e.g. human antibody constant regions or parts thereof. For example, a VL domain may be attached at its C-terminal end to antibody light chain constant domains including human Cκ or Cλ chains. Similarly, a binding member based on a VH domain may be attached at its C-terminal end to all or part (e.g. a CH1 domain) of an immunoglobulin heavy chain derived from any antibody isotype, e.g. IgG, IgA, IgE and IgM and any of the isotype sub-classes, particularly IgG1 and IgG4. IgG1 is advantageous, due to its effector function and ease of manufacture. Any synthetic or other constant region variant that has these properties and stabilizes variable regions may also be useful in the present invention.

Binding members of the invention may be labelled with a detectable or functional label. Thus, a binding member or antibody molecule can be present in the form of an immunoconjugate so as to obtain a detectable and/or quantifiable signal. An immunoconjugates may comprise an antibody molecule of the invention conjugated with detectable or functional label. A label can be any molecule that produces or can be induced to produce a signal, including but not limited to fluorescers, radiolabels, enzymes, chemiluminescers or photosensitizers. Thus, binding may be detected and/or measured by detecting fluorescence or luminescence, radioactivity, enzyme activity or light absorbance.

Suitable labels include, by way of illustration and not limitation,
enzymes, such as alkaline phosphatase, glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase e.g. horseradish peroxidase;
dyes;
fluorescent labels or fluorescers, such as fluorescein and its derivatives, fluorochrome, rhodamine compounds and derivatives, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates e.g. Europium etc (Perkin Elmer and Cis Biointernational),
chemoluminescent labels or chemiluminescers, such as isoluminol, luminol and the dioxetanes;
bio-luminescent labels, such as luciferase and luciferin;
sensitizers;
coenzymes;
enzyme substrates;
radiolabels including but not limited to bromine77, carbon14, cobalt57, fluorine8, gallium67, gallium 68, hydrogen3 (tritium), indium111, indium 113m, iodine123m, iodine125, iodine126, iodine131, iodine133, mercury107, mercury/203, phosphorous32, rhenium99m, rhenium101, rhenium105, ruthenium95, ruthenium97, ruthenium103, ruthenium105, scandium47, selenium75, sulphur35, technetium99, technetium99m, tellurium121m, tellurium122m, tellurium125m, thulium165, thulium167, thulium168, yttrium199 and other radiolabels mentioned herein;
particles, such as latex or carbon particles; metal sol; crystallite; liposomes; cells, etc., which may be further labelled with a dye, catalyst or other detectable group;
molecules such as biotin, digoxygenin or 5-bromodeoxyuridine;
toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

Suitable enzymes and coenzymes are disclosed in Litman, et al., U54275149, and Boguslaski, et al., U54318980, each of which are herein incorporated by reference in their entireties. Suitable fluorescers and chemiluminescers are disclosed in Litman, et al., U54275149, which is incorporated herein by reference in its entirety. Labels further include chemical moieties, such as biotin that may be detected via binding to a specific cognate detectable moiety, e.g. labelled avidin or streptavidin. Detectable labels may be attached to antibodies of the invention using conventional chemistry known in the art.

Immunoconjugates or their functional fragments can be prepared by methods known to the person skilled in the art. They can be coupled to enzymes or to fluorescent labels directly or by the intermediary of a spacer group or of a linking group, such as a polyaldehyde, like glutaraldehyde, ethylenediaminetetraacetic acid (EDTA), diethylene-triaminepentaacetic acid (DPTA), or in the presence of coupling agents, such as those mentioned above for the therapeutic conjugates. Conjugates containing labels of fluorescein type can be prepared by reaction with an isothiocyanate.

The methods known to the person skilled in the art existing for coupling the therapeutic radioisotopes to the antibodies either directly or via a chelating agent, such as EDTA, DTPA mentioned above can be used for the radioelements which can be used in diagnosis. It is likewise possible to perform labelling with sodium125 by the chloramine T method (Hunter W. M. and Greenwood F. C. (1962) Nature 194:495) or else with technetium99m by the technique of Crockford et al., (U.S. Pat. No. 4,424,200, herein incorporated by reference in its entirety) or attached via DTPA as described by Hnatowich (U.S. Pat. No. 4,479,930, herein incorporated by reference in its entirety).

There are numerous methods by which the label can produce a signal detectable by external means, for example, by visual examination, electromagnetic radiation, heat, and chemical reagents. The label can also be bound to another binding member that binds the antibody of the invention, or to a support.

The label can directly produce a signal, and therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a second wavelength. This second wavelength emission may also transfer energy to a labelled acceptor molecule, and the resultant energy dissipated from the acceptor molecule by emission of light for example fluorescence resonance energy transfer (FRET). Other labels that directly produce a signal include radioactive isotopes and dyes.

Alternately, the label may need other components to produce a signal, and the signal producing system would then include all the components required to produce a measurable signal, which may include substrates, coenzymes, enhancers, additional enzymes, substances that react with enzymic products, catalysts, activators, cofactors, inhibitors, scavengers, metal ions, and a specific binding substance required for binding of signal generating substances. A detailed discussion of suitable signal producing systems can be found in Ullman, et al. U.S. Pat. No. 5,185,243, which is herein incorporated herein by reference in its entirety.

The present invention provides a method comprising binding of a binding member as provided herein to IL-6. As noted, such binding may take place in vivo, e.g. following administration of a binding member, or nucleic acid encoding a binding member, or it may take place in vitro, for example in ELISA, Western blotting, immunocytochemistry, immunoprecipitation, affinity chromatography, and biochemical or cell-based assays, such as a TF-1 cell proliferation assay.

The present invention also provides for measuring levels of antigen directly, by employing a binding member according to the invention for example in a biosensor system. For instance, the present invention comprises a method of detecting and/or measuring binding to IL-6, comprising, (i) exposing said binding member to IL-6 and (ii) detecting binding of said binding member to IL-6, wherein binding is detected using any method or detectable label described herein. This, and any other binding detection method described herein, may be interpreted directly by the person performing the method, for instance, by visually observing a detectable label. Alternatively, this method, or any other binding detection method described herein, may produce a report in the form of an autoradiograph, a photograph, a computer printout, a flow cytometry report, a graph, a chart, a test tube or container or well containing the result, or any other visual or physical representation of a result of the method.

The amount of binding of binding member to IL-6 may be determined. Quantitation may be related to the amount of the antigen in a test sample, which may be of diagnostic interest. Screening for IL-6 binding and/or the quantitation thereof may be useful, for instance, in screening patients for diseases or disorders referred to herein and/or any other disease or disorder involving aberrant IL-6 expression and/or activity.

A diagnostic method of the invention may comprise (i) obtaining a tissue or fluid sample from a subject, (ii) exposing said tissue or fluid sample to one or more binding members of the present invention; and (iii) detecting bound IL-6 as compared with a control sample, wherein an increase in the amount of IL-6 binding as compared with the control may indicate an aberrant level of IL-6 expression or activity. Tissue or fluid samples to be tested include blood, serum, urine, biopsy material, tumors, or any tissue suspected of containing aberrant IL-6 levels. Subjects testing positive for aberrant IL-6 levels or activity may also benefit from the treatment methods disclosed later herein.

Those skilled in the art are able to choose a suitable mode of determining binding of the binding member to an antigen according to their preference and general knowledge, in light of the methods disclosed herein.

The reactivities of binding members in a sample may be determined by any appropriate means. Radioimmunoassay (RIA) is one possibility. Radioactive labelled antigen is mixed with unlabelled antigen (the test sample) and allowed to bind to the binding member. Bound antigen is physically separated from unbound antigen and the amount of radioactive antigen bound to the binding member determined. The more antigen there is in the test sample the less radioactive antigen will bind to the binding member. A competitive binding assay may also be used with non-radioactive antigen, using antigen or an analogue linked to a reporter molecule. The reporter molecule may be a fluorochrome, phosphor or laser dye with spectrally isolated absorption or emission characteristics. Suitable fluorochromes include fluorescein, rhodamine, phycoerythrin and Texas Red, and lanthanide chelates or cryptates. Suitable chromogenic dyes include diaminobenzidine.

Other reporters include macromolecular colloidal particles or particulate material, such as latex beads that are colored, magnetic or paramagnetic, and biologically or chemically active agents that can directly or indirectly cause detectable signals to be visually observed, electronically detected or otherwise recorded. These molecules may be enzymes, which catalyze reactions that develop, or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The signals generated by individual binding member-reporter conjugates may be used to derive quantifiable absolute or relative data of the relevant binding member binding in samples (normal and test).

A kit comprising a binding member according to any aspect or embodiment of the present invention is also provided as an aspect of the present invention. In the kit, the binding member may be labelled to allow its reactivity in a sample to be determined, e.g. as described further below. Further the binding member may or may not be attached to a solid support. Components of a kit are generally sterile and in sealed vials or other containers. Kits may be employed in diagnostic analysis or other methods for which binding members are useful. A kit may contain instructions for use of the components in a method, e.g. a method in accordance with the present invention. Ancillary materials to assist in or to enable performing such a method may be included within a kit of the invention. The ancillary materials include a second, different binding member which binds to the first binding member and is conjugated to a detectable label (e.g., a fluorescent label, radioactive isotope or enzyme). Antibody-based kits may also comprise beads for conducting an immunoprecipitation. Each component of the kits is generally in its own suitable container. Thus, these kits generally comprise distinct containers suitable for each binding member. Further, the kits may comprise instructions for performing the assay and methods for interpreting and analyzing the data resulting from the performance of the assay.

The present invention also provides the use of a binding member as above for measuring antigen levels in a competition assay, that is to say a method of measuring the level of antigen in a sample by employing a binding member as provided by the present invention in a competition assay. This may be where the physical separation of bound from unbound antigen is not required. Linking a reporter molecule to the binding member so that a physical or optical change occurs on binding is one possibility. The reporter molecule may directly or indirectly generate detectable signals, which may be quantifiable. The linkage of reporter molecules may be directly or indirectly, covalently, e.g. via a peptide bond or non-covalently. Linkage via a peptide bond may be as a result of recombinant expression of a gene fusion encoding antibody and reporter molecule.

In various aspects and embodiments, the present invention extends to a binding member that competes for binding to IL-6 with any binding member defined herein, e.g. Antibody 18, e.g. in IgG1 format. Competition between binding members may be assayed easily in vitro, for example by tagging a specific reporter molecule to one binding member which can be detected in the presence of other untagged binding member(s), to enable identification of binding members which bind the same epitope or an overlapping epitope. Competition may be determined for example using ELISA in which IL-6 is immobilized to a plate and a first tagged or labelled binding member along with one or more other untagged or unlabelled binding members is added to the plate. Presence of an untagged binding member that competes with the tagged binding member is observed by a decrease in the signal emitted by the tagged binding member.

For example, the present invention includes a method of identifying an IL-6 binding compound, comprising (i) immobilizing IL-6 to a support, (ii) contacting said immobilized IL-6 simultaneously or in a step-wise manner with at least one tagged or labelled binding member according to the invention and one or more untagged or unlabelled test binding compounds, and (iii) identifying a new IL-6 binding compound by observing a decrease in the amount of bound tag from the tagged binding member. Such methods can be performed in a high-throughput manner using a multiwell or array format. Such assays may be also be performed in solution. See, for instance, U.S. Pat. No. 5,814,468, which is herein incorporated by reference in its entirety. As described above, detection of binding may be interpreted directly by the person performing the method, for instance, by visually observing a detectable label, or a decrease in the presence thereof. Alternatively, the binding methods of the invention may produce a report in the form of an autoradiograph, a photograph, a computer printout, a flow cytometry report, a graph, a chart, a test tube or container or well containing the result, or any other visual or physical representation of a result of the method.

Competition assays can also be used in epitope mapping. In one instance epitope mapping may be used to identify the epitope bound by an IL-6 binding member which optionally may have optimized neutralizing and/or modulating characteristics. Such an epitope can be linear or conformational. A conformational epitope can comprise at least two different fragments of IL-6, wherein said fragments are positioned in proximity to each other when IL-6 is folded in its tertiary or quaternary structure to form a conformational epitope which is recognized by an inhibitor of IL-6, such as an IL-6-binding member. In testing for competition a peptide fragment of the antigen may be employed, especially a peptide including or consisting essentially of an epitope of interest. A peptide having the epitope sequence plus one or more amino acids at either end may be used. Binding members according to the present invention may be such that their binding for antigen is inhibited by a peptide with or including the sequence given.

The present invention further provides an isolated nucleic acid encoding a binding member of the present invention. Nucleic acid may include DNA and/or RNA. In one, the present invention provides a nucleic acid that codes for a CDR or set of CDRs or VH domain or VL domain or antibody antigen-binding site or antibody molecule, e.g. scFv or IgG1, of the invention as defined above.

The present invention also provides constructs in the form of plasmids, vectors, transcription or expression cassettes which comprise at least one polynucleotide as above.

The present invention also provides a recombinant host cell that comprises one or more constructs as above. A nucleic acid encoding any CDR or set of CDRs or VH domain or VL domain or antibody antigen-binding site or antibody molecule, e.g. scFv or IgG1 as provided, itself forms an aspect of the present invention, as does a method of production of the encoded product, which method comprises expression from encoding nucleic acid therefor. Expression may conveniently be achieved by culturing under appropriate conditions recombinant host cells containing the nucleic acid. Following production by expression a VH or VL domain, or binding member may be isolated and/or purified using any suitable technique, then used as appropriate.

Nucleic acid according to the present invention may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

A yet further aspect provides a method of production of an antibody VH variable domain, the method including causing expression from encoding nucleic acid. Such a method may comprise culturing host cells under conditions for production of said antibody VH variable domain.

Analogous methods for production of VL variable domains and binding members comprising a VH and/or VL domain are provided as further aspects of the present invention.

A method of production may comprise a step of isolation and/or purification of the product. A method of production may comprise formulating the product into a composition including at least one additional component, such as a pharmaceutically acceptable excipient.

Systems for cloning and expression of a polypeptide in a variety of different host cells are well known. Suitable host cells include bacteria, mammalian cells, plant cells, filamentous fungi, yeast and baculovirus systems and transgenic plants and animals. The expression of antibodies and antibody fragments in prokaryotic cells is well established in the art. For a review, see for example Plückthun (Pluckthun, A. (1991) Bio/Technology 9: 545-551). A common bacterial host is E. coli.

Expression in eukaryotic cells in culture is also available to those skilled in the art as an option for production of a binding member (Chadd H E and Chamow S M (2001) Current Opinion in Biotechnology 12: 188-194; Andersen D C and Krummen L (2002) Current Opinion in Biotechnology 13: 117; Larrick J W and Thomas D W (2001) Current Opinion in Biotechnology 12:411-418) Mammalian cell lines available in the art for expression of a heterologous polypeptide include Chinese hamster ovary (CHO) cells, HeLa cells, baby hamster kidney cells, NS0 mouse melanoma cells, YB2/0 rat myeloma cells, human embryonic kidney cells, human embryonic retina cells and many others.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids e.g. phagemid, or viral e.g. ‘phage, as appropriate (Sambrook and Russell, Molecular Cloning: a Laboratory Manual: 3rd edition, 2001, Cold Spring Harbor Laboratory Press). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. (Ausubel et al. eds., Short Protocols in Molecular Biology: A Compendium of Methods from Current Protocols in Molecular Biology, John Wiley & Sons, 4^{th} edition 1999).

A further aspect of the present invention provides a host cell containing nucleic acid as disclosed herein. Such a host cell may be in vitro and may be in culture. Such a host cell may be in vivo. In vivo presence of the host cell may allow intra-cellular expression of the binding members of the present invention as “intrabodies” or intra-cellular antibodies. Intrabodies may be used for gene therapy.

A still further aspect provides a method comprising introducing nucleic acid of the invention into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques may include calcium chloride transformation, electroporation and transfection using bacteriophage.

The introduction may be followed by causing or allowing expression from the nucleic acid, e.g. by culturing host cells under conditions for expression of the gene. The purification of the expressed product may be achieved by methods known to one of skill in the art.

Nucleic acid of the invention may be integrated into the genome (e.g. chromosome) of the host cell. Integration may be promoted by inclusion of sequences that promote recombination with the genome, in accordance with standard techniques.

The present invention also provides a method that comprises using a construct as stated above in an expression system in order to express a binding member or polypeptide as above.

There is evidence for involvement of IL-6 in a variety of disorders, as discussed elsewhere herein. The binding members of the present invention may therefore be used in a method of diagnosis or treatment of a disorder associated with IL-6. Such a disorder may for example be an inflammatory and/or autoimmune disorder such as for example, rheumatoid arthritis, osteoarthritis, cachexia, chronic obstructive pulmonary disease (COPD), Juvenile idiopathic arthritis, asthma, systemic lupus erythematosus, inflammatory bowel disease, Crohn's disease or atherosclerosis. A binding member of the present invention may also be used to treat a disorder such as a tumor and/or cancer. Furthermore, a binding member of the present invention may be used to treat and/or prevent pain resulting from or associated with the diseases and conditions listed herein. Binding members of the present invention may also be used in method of diagnosis or treatment of at least one IL-6 related disease, in a patient, animal, organ, tissue or cell, including, but not limited to: obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute-, allergic-, atrophic rhinitis and chronic rhinitis including rhinitis caseosa, hypertrophic rhinitis, rhinitis pumlenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis, sinusitis, idiopathic pulmonary fibrosis (IPF); sarcoidosis, farmer's lung and related diseases, adult respiratory distress syndrome, hypersensitivity pneumonitis, fibroid lung and idiopathic interstitial pneumonia; rheumatoid arthritis, juvenile chronic arthritis, systemic onset juvenile arthritis, seronegative spondyloarthropathies (including ankylosing spondylitis, psoriatic arthritis and Reiter's disease), Behcet's disease, Siogren's syndrome and systemic sclerosis, gout, osteoporosis and osteoarthritis; psoriasis, atopical dermatitis, contact dermatitis and other eczmatous dermatoses, allergic contact dermatitis, seborrhoetic dermatitis, Lichen planus, scleroderma, Pemphigus, bullous pemphigoid, Epidermolysis bullosa, urticaria, angiodermas, vasculitides, erythemas, cutaneous eosinophilias, uveitis, Alopecia areata, allergic conjunctivitis and vernalvemal conjunctivitis; (gastrointestinal tract) gastric ulcer, Coeliac disease, proctitis, eosinopilic gastro-enteritis, mastocytosis, inflammatory bowel disease, Crohn's disease, ulcerative colitis, antiphospholipid syndrome)), food-related allergies which have effects remote from the gut, e.g., migraine, rhinitis and eczema; cachexia, multiple sclerosis, atherosclerosis, Acquired Immunodeficiency Syndrome (AIDS), mesangial proliferative glomerulonephritis, nephrotic syndrome, nephritis, glomerular nephritis, acute renal failure, hemodialysis, uremia, localised or discoid lupus erythematosus, systemic lupus erythematosus, Castleman's Disease, Hashimoto's thyroiditis, myasthenia gravis, type I diabetes, type B insulin-resistant diabetes, sickle cell anaemia, iridocyclitis/uveitis/optic neuritis, nephritic syndrome, eosinophilia fascitis, hyper IgE syndrome, systemic vasculitis/wegener's granulomatosis, orchitis/vasectomy reversal procedures, lepromatous leprosy, alcohol-induced hepatitis, sezary syndrome and idiopathic thrombocytopenia purpura; post-operative adhesions, nephrosis, systemic inflammatory response syndrome, sepsis syndrome, gram positive sepsis, gram negative sepsis, culture negative sepsis, fungal sepsis, neutropenic fever, acute pancreatitis, urosepsis, Graves disease, Raynaud's disease, antibody-mediatated cytotoxicity, type III hypersensitivity reactions, POEMS syndrome (polyneuropathy, organomegaly, endocrinopathy, monoclonal gammopathy, and skin changes syndrome), mixed connective tissue disease, idiopathic Addison's disease, diabetes mellitus, chronic active hepatitis, primary billiary cirrhosis, vitiligo, post-MI (cardiotomy) syndrome, type IV hypersensitivity, granulomas due to intracellular organisms, Wilson's disease, hemachromatosis, alpha-I-antitrypsin deficiency, diabetic retinopathy, hashimoto's thyroiditis, hypothalamic-pituitary-adrenal axis evaluation, thyroiditis, encephalomyelitis, neonatal chronic lung disease, familial hematophagocytic lymphohistiocytosis, alopecia, radiation therapy (e.g., including but not limited to asthenia, anemia, cachexia, and the like), chronic salicylate intoxication, sleep apnea, obesity, heart failure, and meningococcemia; acute and chronic following, for example, transplantation of kidney, heart, liver, lung, pancreas, bone marrow, bone, small bowel, skin, cartilage and cornea; and chronic graft versus host disease; leukaemia, acute lymphoblastic leukaemia (ALL), acute leukaemia, T-cell, B-cell, or FAB ALL, chromic myelocytic leukaemia (CIVIL), acute myeloid leukaemia (AML), chronic lymphocytic leukaemia (CLL), hairy cell leukaemia, myelodyplastic syndrome (MDS), any lymphoma, Hodgkin's disease, non-hodgkin's lymphoma, any malignant lymphoma, Burkitt's lymphoma, multiple myeloma, Kaposi's sarcoma, renal cell carcinoma, colorectal carcinoma, prostatic carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, cancer related bone resorption, cancer related bone pain; the suppression of cancer metastasis; the amelioration of cancer cachexia; Cystic fibrosis, stroke, re-perfusion injury in the heart, brain, peripheral limbs and other organs; Burn wounds, trauma/haemorrhage, ionizing radiation exposure, chronic skin ulcers; reproductive organ diseases (e.g. disorders of ovulation, menstruation and implantation, pre-term labour, pre-eclampsia, endometriosis); acute or chronic bacterial infection, acute and chronic parasitic or infectious processes, including bacterial, viral and fungal infections, HIV infection/HIV neuropathy, meningitis, hepatitis (A, B or C, or other viral hepatitis the like), septic arthritis, peritonitis, pneumonia, epiglottitis, e. coli 0157: h7, hemolytic uremic syndrome/thrombotic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, Lyme disease, influenza A, epstein-barr virus, vital-associated hemaphagocytic syndrome, viral encephalitis/aseptic meningitis, depression and the like. Accordingly, the invention provides a method of treating an IL-6 related disorder, comprising administering to a patient in need thereof an effective amount of one or more binding members of the present invention alone or in a combined therapeutic regimen with another appropriate medicament known in the art or described herein.

In one embodiment the IL-6 related disorder is depression—also referred to herein as major depressive disorder. Major depressive disorder (also known as and referred to herein as clinical depression, major depression, unipolar depression, or unipolar disorder) is a mental disorder characterized by an all-encompassing low mood accompanied by low self-esteem, and loss of interest or pleasure in normally enjoyable activities. The term “major depressive disorder” was selected by the American Psychiatric Association to designate this symptom cluster as a mood disorder in the 1980 version of the Diagnostic and Statistical Manual of Mental Disorders (DSM-III) classification, and has become widely used since. The general term depression is often used to denote the disorder, but as it can also be used in reference to other types of psychological depression, more precise terminology is preferred for the disorder in clinical and research use. Major depression is a disabling condition which adversely affects a person's family, work or school life, sleeping and eating habits, and general health.

Depression is highly comorbid with diseases involving systemic inflammation. Systemic inflammation is observed in many depressed patients, as reflected in elevated plasma bio-markers of inflammation. Additionally, activated cytokine signaling pathways may be detected in blood and CSF of depressed patients. Moreover, cytokines (IFN-a, IL-2) may induce symptoms of major depressive disorder in medically ill patients with no history of psychiatric illness. Accordingly, the invention provides a method of treating a depression, comprising administering to a patient in need thereof an effective amount of one or more binding members of the present invention alone or in a combined therapeutic regimen with another appropriate medicament known in the art, e.g., anti-depressants, such as selective serotonin reuptake inhibitors (SSRIs), such as sertraline, escitalopram, fluoxetine, paroxetine, and citalopram; or described herein.

The binding members of the invention also have analgesic properties. As such, they are appropriate as analgesics for treating and/or preventing pain associated with the diseases listed herein as well as chronic and acute pain resulting from or associated with wounds, medical procedures, surgeries, injury, trauma, etc. For example, the binding members may be used as analgics post-surgery analgesics. They may also be used to treat or prevent pain resulting from or associated with ankylosing spondylitis, inflammatory lower back pain, neuropathic pain, painful neuroma, fibromyalgia, headaches, e.g., chronic head aches and migraines, pancreatitis, spinal nerve compression syndromes and non-malignant skeletal pain, inflammatory osteoarthritic pain, rheumatoid arthritic pain, cancer pain, e.g., bone cancer pain.

The binding members of the invention also may be used to treat pulmonary hypertension associated with several diseases such as, but not limited to, COPD, scleroderma, systemic lupus erythematosus, POEMs as well as idiopathic pulmonary hypertension. Elevated IL-6 levels have been reported in patients with pulmonary hypertension associated with many of these conditions (Savale, L. et al Respir. Res. (2009) 10, 6 and references therein; Steiner, M. K. et al Circ. Res. (2009) 104(2) 236-244 and references therein). IL-6-deficient mice exposed to hypoxia show reduced right ventricular systolic blood pressure and reduced right ventricular hypertrophy when compared to WT mice exposed to hypoxia (Savale, L. et al Respir. Res. (2009) 10, 6 and references therein). Furthermore, IL-6-overexpressing transgenic mice develop enhanced right ventricular hypertrophy and enhanced right ventricular systolic blood pressure under hypoxic conditions when compared to non-transgenic controls (Steiner, M. K. et al Circ. Res. (2009) 104(2) 236-244 and references therein) and exogenously administered IL-6 aggravates the development of pulmonary hypertension in mice exposed to chronic hypoxia (Golembesk) S. M. et al Chest (2005) 128(6 Suppl) 572S-573S).

Further, patients with stable COPD have been observed to have increased serum levels of IL-6 over healthy controls (Yanbaeva, D. G. et al BMC Med Genet (2009) 10, 23; Savale, L. et al Am. J. Respir. Crit Care Med. (2009) 179(7), 566-571; Eickhoff P. et al Am. J. Respir. Crit Care Med. (2008) 178(12) 1211-1218). Enhanced IL-6 levels have been associated with impaired lung function in COPD patients (R. E. et al Chest (2008) 133(1) 19-25; Thorleifesson, S. J. et al Respir. Med. (2009) 103(10) 1548-1553). Several studies have also reported increased levels of IL-6 in sputum and/or serum at the onset of COPD exacerbations when compared with IL-6 levels measured at the resolution of the exacerbation or IL-6 levels measured in stable COPD patients (Valipour, A. et al Clinical Science (2008) 115(7), 225-232; Groenewegen, K. H. et al Respir. Med. (2007) 101(11) 2409-2415; Perera, W. R. et al Eur. Respir. J. (2007) 29(3), 527-534) Enhanced IL-6 levels have also been associated with more frequent exacerbators (Bhowmik, A. et al Thorax (2000) 55(2) 114-120). Treatment of mice with anti-IL-6 antibodies or mice deficient in IL-6 show reduced pulmonary inflammation in certain animal models, for example ozone-induced pulmonary inflammation and bleomycin-induced pulmonary inflammation and fibrosis (Saito, F. et al Am. J. Respir. Cell Mol. Biol. (2008) 38(5) 566-571; Lang, J. E. et al Am. J. Physiol. Lung Cell Mol. Physiol. (2008) 294(5) L1013-L1020; Johnston, R. A. et al Am. J. Physiol. Lung Cell Mol. Physiol. (2005) 288(2) L390-L397). Higher IL-6 levels have also been associated with certain co-morbidities of COPD, for example pulmonary hypertension (Chaouat, A. et al Chest (2009) 136(3) 678-687; Eddahibi, S. et al Proceedings of the American Thoracic Society (2006) 3(6), 475-476).

Evidence for involvement of IL-6 in certain other disorders is well understood. The data presented herein and in PCT Publication No. WO 2008/065378 further indicates that binding members of the invention can be used to treat such disorders, including preventative treatment and reduction of severity of the disorders. Accordingly, the invention provides a method of treating or reducing the severity of at least one symptom of any of the disorders mentioned herein, comprising administering to a patient in need thereof an effective amount of one or more binding members of the present invention alone or in a combined therapeutic regimen with another appropriate medicament known in the art or described herein such that the severity of at least one symptom of any of the above disorders is reduced.

Thus, the binding members of the present invention are useful as therapeutic agents in the treatment of diseases or disorders involving IL-6 and/or IL-6Ra expression and/or activity, especially aberrant expression/activity. A method of treatment may comprise administering an effective amount of a binding member of the invention to a patient in need thereof, wherein aberrant expression and/or activity of IL-6 and/or IL-6Ra is decreased. A method of treatment may comprise (i) identifying a patient demonstrating aberrant IL-6:IL-6Ra levels or activity, for instance using the diagnostic methods described above, and (ii) administering an effective amount of a binding member of the invention to the patient, wherein aberrant expression and/or activity of IL-6Ra and/or IL-6 is decreased. An effective amount according to the invention is an amount that decreases the aberrant expression and/or activity of IL-6 and/or IL-6Ra so as to decrease or lessen the severity of at least one symptom of the particular disease or disorder being treated, but not necessarily cure the disease or disorder.

The invention also provides a method of antagonising at least one effect of IL-6, comprising contacting with or administering an effective amount of one or more binding members of the present invention such that said at least one effect of IL-6 is antagonised. Effects of IL-6 that may be antagonised by the methods of the invention include IL-6 binding to gp130, and downstream effects that arise as a consequence of this binding.

Accordingly, further aspects of the invention provide methods of treatment comprising administration of a binding member as provided, pharmaceutical compositions comprising such a binding member, and use of such a binding member in the manufacture of a medicament for administration, for example in a method of making a medicament or pharmaceutical composition comprising formulating the binding member with a pharmaceutically acceptable excipient. A pharmaceutically acceptable excipient may be a compound or a combination of compounds entering into a pharmaceutical composition not provoking secondary reactions and which allows, for example, facilitation of the administration of the active compound(s), an increase in its lifespan and/or in its efficacy in the body, an increase in its solubility in solution or else an improvement in its conservation. These pharmaceutically acceptable vehicles are well known and will be adapted by the person skilled in the art as a function of the nature and of the mode of administration of the active compound(s) chosen.

Binding members of the present invention will usually be administered in the form of a pharmaceutical composition, which may comprise at least one component in addition to the binding member. Thus pharmaceutical compositions according to the present invention, and for use in accordance with the present invention, may comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, inhaled, intra-tracheal, topical, intra-vesicular or by injection, as discussed below.

The present invention relates to sterile, stable pharmaceutical formulations comprising an antibody of the invention.

The present invention provides methods of stabilizing an antibody of the invention.

The present invention further relates to processes of making a sterile, stable formulation comprising an antibody of the invention.

All formulations of antibodies of the invention described herein are collectively referred to as “formulations of the invention”, “liquid formulations of the invention”, “high concentration stable liquid formulations of the invention”, “antibody liquid formulations of the invention”, “reconstituted liquid formulations of the invention” or “antibody formulations of the invention”.

The phrase “pharmaceutically acceptable” as used herein means approved by a regulatory agency of the Federal or a state government, or listed in the U.S. Pharmacopeia, European Pharmacopia or other generally recognized pharmacopeia for use in animals, and more particularly in humans.

The terms “stability” and “stable” as used herein in the context of a liquid formulation comprising an antibody (including antibody fragment thereof) of the invention refer to the resistance of the antibody (including antibody fragment thereof) in the formulation to aggregation, degradation or fragmentation under given manufacture, preparation, transportation and storage conditions. The “stable” formulations of the invention retain biological activity under given manufacture, preparation, transportation and storage conditions. The stability of said antibody (including antibody fragment thereof) can be assessed by degrees of aggregation, degradation or fragmentation, as measured by HPSEC, reverse phase chromatography, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of an antibody (including antibody fragment thereof) in histidine, pH 6.0-6.5 and optionally one or more excipient, which reference formulation regularly gives a single monomer peak (e.g., ≧97% area) by HPSEC. The overall stability of a formulation comprising an antibody (including antibody fragment thereof) can be assessed by various immunological assays including, for example, ELISA and radioimmunoassay using isolated antigen molecules.

The phrase “low to undetectable levels of aggregation” as used herein refers to samples containing no more than about 5%, no more than about 4%, no more than about 3%, no more than about 2%, no more than about 1% and no more than about 0.5% aggregation by weight of protein as measured by high performance size exclusion chromatography (HPSEC) or static light scattering (SLS) techniques.

The term “low to undetectable levels of fragmentation” as used herein refers to samples containing equal to or more than about 80%, about 85%, about 90%, about 95%, about 98% or about 99% of the total protein, for example, in a single peak as determined by HPSEC or reverse phase echromatography, or in two peaks (e.g., heavy- and light-chains) (or as many peaks as there are subunits) by reduced Capillary Gel Electrophoresis (rCGE), representing the non-degraded antibody or a non-degraded fragment thereof, and containing no other single peaks having more than about 5%, more than about 4%, more than about 3%, more than about 2%, more than about 1%, or more than about 0.5% of the total protein in each. The term “reduced Capillary Gel Electrophoresis” as used herein refers to capillary gel electrophoresis under reducing conditions sufficient to reduce disulfide bonds in an antibody.

The present invention relates to stable, high concentration formulations of antibodies of the invention. In one embodiment, a formulation of the invention is a liquid formulation. In another embodiment, a formulation of the invention is a lyophilized formulation. In a further embodiment, a formulation of the invention is a reconstituted liquid formulation.

In one embodiment, a formulation of the invention is a stable liquid formulation. In one embodiment, a liquid formulation of the invention is an aqueous formulation. In a specific embodiment, a liquid formulation of the invention is an aqueous formulation wherein the aqueous carrier is distilled water.

In one embodiment, a formulation of the invention is sterile.

In one embodiment, a formulation of the invention is homogeneous.

In one embodiment, a formulation of the invention is isotonic.

The invention encompasses stable liquid formulations comprising a single antibody of interest (including antibody fragment thereof), for example, an antibody that specifically binds to IL-6. The invention also encompasses stable liquid formulations comprising two or more antibodies of interest (including antibody fragments thereof), for example, antibodies that specifically bind to IL-6 polypeptide(s).

In one embodiment, a formulation of the invention comprises at least about 1 mg/ml, at least about 5 mg/ml, at least about 10 mg/ml, at least about 20 mg/ml, at least about 30 mg/ml, at least about 40 mg/ml, at least about 50 mg/ml, at least about 60 mg/ml, at least about 70 mg/ml, at least about 80 mg/ml, at least about 90 mg/ml, at least about 100 mg/ml, at least about 110 mg/ml, at least about 120 mg/ml, at least about 130 mg/ml, at least about 140 mg/ml, at least about 150 mg/ml, at least about 160 mg/ml, at least about 170 mg/ml, at least about 180 mg/ml, at least about 190 mg/ml, at least about 200 mg/ml, at least about 250 mg/ml, or at least about 300 mg/ml of an anti-IL-6 antibody of the invention. In a specific embodiment, a formulation of the invention comprises at least about 100 mg/ml of an anti-IL-6 antibody of the invention. In a specific embodiment, a formulation of the invention comprises at least about 125 mg/ml of an anti-IL-6 antibody of the invention. In a specific embodiment, a formulation of the invention comprises at least about 130 mg/ml of an anti-IL-6 antibody of the invention. In a specific embodiment, a formulation of the invention comprises at least about 150 mg/ml of an anti-IL-6 antibody of the invention. In a specific embodiment, a formulation of the invention comprises at least about 90 mg/ml of an anti-IL-6 antibody of the invention. In another embodiment, a formulation of the invention comprises between about 1 mg/ml and about 25 mg/ml, between about 1 mg/ml and about 200 mg/ml, between about 25 mg/ml and about 200 mg/ml, between about 50 mg/ml and about 200 mg/ml, between about 75 mg/ml and about 200 mg/ml, between about 100 mg/ml and about 200 mg/ml, between about 125 mg/ml and about 200 mg/ml, between about 150 mg/ml and about 200 mg/ml, between about 25 mg/ml and about 150 mg/ml, between about 50 mg/ml and about 150 mg/ml, between about 75 mg/ml and about 150 mg/ml, between about 100 mg/ml and about 150 mg/ml, between about 125 mg/ml and about 150 mg/ml, between about 25 mg/ml and about 125 mg/ml, between about 50 mg/ml and about 125 mg/ml, between about 75 mg/ml and about 125 mg/ml, between about 100 mg/ml and about 125 mg/ml, between about 25 mg/ml and about 100 mg/ml, between about 50 mg/ml and about 100 mg/ml, between about 75 mg/ml and about 100 mg/ml, between about 25 mg/ml and about 75 mg/ml, between about 50 mg/ml and about 75 mg/ml, or between about 25 mg/ml and about 50 mg/ml of an anti-IL-6 antibody of the invention. In a specific embodiment, a formulation of the invention comprises between about 90 mg/ml and about 110 mg/ml of an anti-IL-6 antibody of the invention. In a specific embodiment, a formulation of the invention comprises between about 100 mg/ml and about 210 mg/ml of an anti-IL-6 antibody of the invention. In a further embodiment, a formulation described herein comprises about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 250 mg/ml, or about 300 mg/ml of an anti-IL-6 antibody of the invention. In a specific embodiment, a formulation of the invention comprises about 100 mg/ml of an anti-IL-6 antibody of the invention. In a specific embodiment, a formulation of the invention comprises about 125 mg/ml of an anti-IL-6 antibody of the invention. In a specific embodiment, a formulation of the invention comprises about 130 mg/ml of an anti-IL-6 antibody of the invention. In a specific embodiment, a formulation of the invention comprises about 150 mg/ml of an anti-IL-6 antibody of the invention. In a specific embodiment, a formulation of the invention comprises about 200 mg/ml of an anti-IL-6 antibody of the invention.

In one embodiment, a formulation of the invention comprises at least 1 mg/ml, at least 5 mg/ml, at least 10 mg/ml, at least 20 mg/ml, at least 30 mg/ml, at least 40 mg/ml, at least 50 mg/ml, at least 60 mg/ml, at least 70 mg/ml, at least 80 mg/ml, at least 90 mg/ml, at least 100 mg/ml, at least 110 mg/ml, at least 120 mg/ml, at least 130 mg/ml, at least 140 mg/ml, at least 150 mg/ml, at least 160 mg/ml, at least 170 mg/ml, at least 180 mg/ml, at least 190 mg/ml, at least 200 mg/ml, at least 250 mg/ml, or at least 300 mg/ml of an anti-IL-6 antibody of the invention. In a specific embodiment, a formulation of the invention comprises at least 100 mg/ml of an anti-IL-6 antibody of the invention. In a specific embodiment, a formulation of the invention comprises at least 125 mg/ml of an anti-IL-6 antibody of the invention. In a specific embodiment, a formulation of the invention comprises at least 150 mg/ml of an anti-IL-6 antibody of the invention. In a specific embodiment, a formulation of the invention comprises at least 175 mg/ml of an anti-IL-6 antibody of the invention. In a specific embodiment, a formulation of the invention comprises at least 200 mg/ml of an anti-IL-6 antibody of the invention. In another embodiment, a formulation of the invention comprises between 1 mg/ml and 25 mg/ml, between 1 mg/ml and 200 mg/ml, between 25 mg/ml and 200 mg/ml, between 50 mg/ml and 200 mg/ml, between 75 mg/ml and 200 mg/ml, between 100 mg/ml and 200 mg/ml, between 125 mg/ml and 200 mg/ml, between 150 mg/ml and 200 mg/ml, between 25 mg/ml and 150 mg/ml, between 50 mg/ml and 150 mg/ml, between 75 mg/ml and 150 mg/ml, between 100 mg/ml and 150 mg/ml, between 125 mg/ml and 150 mg/ml, between 25 mg/ml and 125 mg/ml, between 50 mg/ml and 125 mg/ml, between 75 mg/ml and 125 mg/ml, between 100 mg/ml and 125 mg/ml, between 25 mg/ml and 100 mg/ml, between 50 mg/ml and 100 mg/ml, between 75 mg/ml and 100 mg/ml, between 25 mg/ml and 75 mg/ml, between 50 mg/ml and 75 mg/ml, or between 25 mg/ml and 50 mg/ml of an anti-IL-6 antibody of the invention. In a specific embodiment, a formulation of the invention comprises between 90 mg/ml and 110 mg/ml of an anti-IL-6 antibody of the invention. In a specific embodiment, a formulation of the invention comprises between 100 mg/ml and 210 mg/ml of an anti-IL-6 antibody of the invention. In a further embodiment, a formulation described herein comprises 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 250 mg/ml, or 300 mg/ml of an anti-IL-6 antibody of the invention. In a specific embodiment, a formulation of the invention comprises 100 mg/ml of an anti-IL-6 antibody of the invention. In a specific embodiment, a formulation of the invention comprises 125 mg/ml of an anti-IL-6 antibody of the invention. In a specific embodiment, a formulation of the invention comprises 150 mg/ml of an anti-IL-6 antibody of the invention. In a specific embodiment, a formulation of the invention comprises 175 mg/ml of an anti-IL-6 antibody of the invention. In a specific embodiment, a formulation of the invention comprises 200 mg/ml of an anti-IL-6 antibody of the invention.

Optionally, the formulations of the invention may further comprise common excipients and/or additives such as buffering agents, saccharides, salts and surfactants. Additionally or alternatively, the formulations of the invention may further comprise common excipients and/or additives, such as, but not limited to, solubilizers, diluents, binders, stabilizers, salts, lipophilic solvents, amino acids, chelators, preservatives, or the like.

In certain embodiments, the buffering agent is selected from the group consisting of histidine, citrate, phosphate, glycine, and acetate. In other embodiments the saccharide excipient is selected from the group consisting of trehalose, sucrose, mannitol, maltose and raffinose. In still other embodiments the surfactant is selected from the group consisting of polysorbate 20, polysorbate 40, polysorbate 80, and Pluronic F68. In yet other embodiments the salt is selected from the group consisting of NaCl, KCl, MgCl2, and CaCl2

Optionally, the formulations of the invention may further comprise other common auxiliary components, such as, but not limited to, suitable excipients, polyols, solubilizers, diluents, binders, stabilizers, lipophilic solvents, chelators, preservatives, or the like.

The formulations of the invention include a buffering or pH adjusting agent to provide improved pH control. In one embodiment, a formulation of the invention has a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, a formulation of the invention has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a formulation of the invention has a pH of about 6.0.

The formulations of the invention include a buffering or pH adjusting agent to provide improved pH control. In one embodiment, a formulation of the invention has a pH of between 3.0 and 9.0, between 4.0 and 8.0, between 5.0 and 8.0, between 5.0 and 7.0, between 5.0 and 6.5, between 5.5 and 8.0, between 5.5 and 7.0, or between 5.5 and 6.5. In a further embodiment, a formulation of the invention has a pH of 3.0, 3.5, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.5, 8.0, 8.5, or 9.0. In a specific embodiment, a formulation of the invention has a pH of 6.0. One of skill in the art understands that the pH of a formulation generally should not be equal to the isoelectric point of the particular antibody (including antibody fragment thereof) to be used in the formulation.

Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the invention as buffering agents include, but are not limited to, glycine and histidine. In certain embodiments, the buffering agent is selected from the group consisting of histidine, citrate, phosphate, glycine, and acetate. In a specific embodiment, the buffering agent is histidine. In another specific embodiment, the buffering agent is citrate. The purity of the buffering agent should be at least 98%, or at least 99%, or at least 99.5%. As used herein, the term “purity” in the context of histidine refers to chemical purity of histidine as understood in the art, e.g., as described in The Merck Index, 13th ed., O'Neil et al. ed. (Merck & Co., 2001).

Buffering agents are typically used at concentrations between about 1 mM and about 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2nd Edition, Chapter 5, p. 194, De Luca and Boylan, “Formulation of Small Volume Parenterals”, Table 5: Commonly used additives in Parenteral Products. In one embodiment, the buffering agent is at a concentration of about 1 mM, or of about 5 mM, or of about 10 mM, or of about 15 mM, or of about 20 mM, or of about 25 mM, or of about 30 mM, or of about 35 mM, or of about 40 mM, or of about 45 mM, or of about 50 mM, or of about 60 mM, or of about 70 mM, or of about 80 mM, or of about 90 mM, or of about 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between about 5 mM and about 50 mM. In another specific embodiment, the buffering agent is at a concentration of between 5 mM and 20 mM.

In a further embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between 5 mM and 50 mM. In another specific embodiment, the buffering agent is at a concentration of between 5 mM and 20 mM.

In certain embodiments, a formulation of the invention comprises a buffering agent. In one embodiment, said buffering agent is selected from the group consisting of histidine, citrate, phosphate, glycine, and acetate. In a specific embodiment, a formulation of the invention comprises histidine as a buffering agent.

In one embodiment, a formulation of the invention comprises at least about 1 mM, at least about 5 mM, at least about 10 mM, at least about 20 mM, at least about 30 mM, at least about 40 mM, at least about 50 mM, at least about 75 mM, at least about 100 mM, at least about 150 mM, or at least about 200 mM histidine. In another embodiment, a formulation of the invention comprises between about 1 mM and about 200 mM, between about 1 mM and about 150 mM, between about 1 mM and about 100 mM, between about 1 mM and about 75 mM, between about 10 mM and about 200 mM, between about 10 mM and about 150 mM, between about 10 mM and about 100 mM, between about 10 mM and about 75 mM, between about 10 mM and about 50 mM, between about 10 mM and about 40 mM, between about 10 mM and about 30 mM, between about 20 mM and about 75 mM, between about 20 mM and about 50 mM, between about 20 mM and about 40 mM, or between about 20 mM and about 30 mM histidine. In a further embodiment of the invention comprises about 1 mM, about 5 mM, about 10 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, or about 200 mM histidine. In a specific embodiment, a formulation of the invention comprises about 10 mM histidine.

In one embodiment, a formulation of the invention comprises at least 1 mM, at least 5 mM, at least 10 mM, at least 20 mM, at least 30 mM, at least 40 mM, at least 50 mM, at least 75 mM, at least 100 mM, at least 150 mM, or at least 200 mM histidine. In another embodiment, a formulation of the invention comprises between 1 mM and 200 mM, between 1 mM and 150 mM, between 1 mM and 100 mM, between 1 mM and 75 mM, between 10 mM and 200 mM, between 10 mM and 150 mM, between 10 mM and 100 mM, between 10 mM and 75 mM, between 10 mM and 50 mM, between 10 mM and 40 mM, between 10 mM and 30 mM, between 20 mM and 75 mM, between 20 mM and 50 mM, between 20 mM and 40 mM, or between 20 mM and 30 mM histidine. In a further embodiment of the invention comprises 1 mM, 5 mM, 10 mM, 20 mM, 25 mM, 30 mM, 35 mM, 40 mM, 45 mM, 50 mM, 60 mM, 70 mM, 80 mM, 90 mM, 100 mM, 150 mM, or 200 mM histidine. In a specific embodiment, a formulation of the invention comprises 10 mM histidine.

In certain embodiments, the formulations of the invention comprise a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume. In one embodiment, the carbohydrate excipient is present at between about 0.1% to about 20%. In another embodiment, the carbohydrate excipient is present at between about 0.1% to about 15%. In a specific embodiment, the carbohydrate excipient is present at between about 0.1% to about 5%, or between about 1% to about 20%, or between about 5% to about 15%, or between about 8% to about 10%, or between about 10% and about 15%, or between about 15% and about 20%. In another specific embodiment, the carbohydrate excipient is present at between 0.1% to 20%, or between 5% to 15%, or between 8% to 10%, or between 10% and 15%, or between 15% and 20%. In still another specific embodiment, the carbohydrate excipient is present at between about 0.1% to about 5%. In still another specific embodiment, the carbohydrate excipient is present at between about 5% to about 10%. In yet another specific embodiment, the carbohydrate excipient is present at between about 15% to about 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

In certain embodiments, the formulations of the invention comprise a carbohydrate excipient. Carbohydrate excipients can act, e.g., as viscosity enhancing agents, stabilizers, bulking agents, solubilizing agents, and/or the like. Carbohydrate excipients are generally present at between 1% to 99% by weight or volume. In one embodiment, the carbohydrate excipient is present at between 0.1% to 20%. In another embodiment, the carbohydrate excipient is present at between 0.1% to 15%. In a specific embodiment, the carbohydrate excipient is present at between 0.1% to 5%, or between 1% to 20%, or between 5% to 15%, or between 8% to 10%, or between 10% and 15%, or between 15% and 20%. In another specific embodiment, the carbohydrate excipient is present at between 0.1% to 20%, or between 5% to 15%, or between 8% to 10%, or between 10% and 15%, or between 15% and 20%. In still another specific embodiment, the carbohydrate excipient is present at between 0.1% to 5%. In still another specific embodiment, the carbohydrate excipient is present at between 5% to 10%. In yet another specific embodiment, the carbohydrate excipient is present at between 15% to 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

Carbohydrate excipients suitable for use in the formulations of the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In one embodiment, the carbohydrate excipients for use in the present invention are selected from the group consisting of, sucrose, trehalose, lactose, mannitol, and raffinose. In a specific embodiment, the carbohydrate excipient is trehalose. In another specific embodiment, the carbohydrate excipient is mannitol. In yet another specific embodiment, the carbohydrate excipient is sucrose. In still another specific embodiment, the carbohydrate excipient is raffinose. The purity of the carbohydrate excipient should be at least 98%, or at least 99%, or at least 99.5%.

In one embodiment, a formulation of the invention comprises at least about 1%, at least about 2%, at least about 4%, at least about 8%, at least about 20%, at least about 30%, or at least about 40% trehalose. In another embodiment, a formulation of the invention comprises between about 1% and about 40%, between about 1% and about 30%, between about 1% and about 20%, between about 2% and about 40%, between about 2% and about 30%, between about 2% and about 20%, between about 4% and about 40%, between about 4% and about 30%, or between about 4% and about 20% trehalose. In a further embodiment, a formulation of the invention comprises about 1%, about 2%, about 4%, about 8%, about 20%, about 30%, or about 40% trehalose. In a specific embodiment, a formulation of the invention comprises about 4% trehalose.

In one embodiment, a formulation of the invention comprises at least 1%, at least 2%, at least 4%, at least 8%, at least 20%, at least 30%, or at least 40% trehalose. In another embodiment, a formulation of the invention comprises between 1% and 40%, between 1% and 30%, between 1% and 20%, between 2% and 40%, between 2% and 30%, between 2% and 20%, between 4% and 40%, between 4% and 30%, or between 4% and 20% trehalose. In a further embodiment, a formulation of the invention comprises 1%, 2%, 4%, 8%, 20%, 30%, or 40% trehalose.

In one embodiment, a formulation of the invention comprises an excipient. In a specific embodiment, a formulation of the invention comprises at least one excipient selected from the group consisting of: sugar, salt, surfactant, amino acid, polyol, chelating agent, emulsifier and preservative. In one embodiment, a formulation of the invention comprises a salt. In one embodiment, a formulation of the invention comprises a salt selected from the group consisting of: NaCl, KCl, CaCl₂, and MgCl₂. In a specific embodiment, a formulation of the invention comprises NaCl.

In one embodiment, a formulation of the invention comprises at least about 10 mM, at least about 25 mM, at least about 50 mM, at least about 75 mM, at least about 80 mM, at least about 100 mM, at least about 125 mM, at least about 150 mM, at least about 175 mM. at least about 200 mM, or at least about 300 mM sodium chloride. In a further embodiment, a formulation described herein comprises between about 10 mM and about 300 mM, between about 10 mM and about 200 mM, between about 10 mM and about 175 mM, between about 10 mM and about 150 mM, between about 25 mM and about 300 mM, between about 25 mM and about 200 mM, between about 25 mM and about 175 mM, between about 25 mM and about 150 mM, between about 50 mM and about 300 mM, between about 50 mM and about 200 mM, between about 50 mM and about 175 mM, between about 50 mM and about 150 mM, between about 75 mM and about 300 mM, between about 75 mM and about 200 mM, between about 75 mM and about 175 mM, between about 75 mM and about 150 mM, between about 100 mM and about 300 mM, between about 100 mM and about 200 mM, between about 100 mM and about 175 mM, or between about 100 mM and about 150 mM sodium chloride. In a further embodiment, a formulation of the invention comprises about 10 mM. about 25 mM, about 50 mM, about 75 mM, about 80 mM, about 100 mM, about 125 mM, about 150 mM, about 175 mM, about 200 mM, or about 300 mM sodium chloride.

In one embodiment, a formulation of the invention comprises at least 10 mM, at least 25 mM, at least 50 mM, at least 75 mM, at least 80 mM, at least 100 mM, at least 125 mM, at least 150 mM, at least 175 mM, at least 200 mM, or at least 300 mM sodium chloride. In a further embodiment, a formulation described herein comprises between 10 mM and 300 mM, between 10 mM and 200 mM, between 10 mM and 175 mM, between 10 mM and 150 mM, between 25 mM and 300 mM, between 25 mM and 200 mM, between 25 mM and 175 mM, between 25 mM and 150 mM, between 50 mM and 300 mM, between 50 mM and 200 mM, between 50 mM and 175 mM, between 50 mM and 150 mM, between 75 mM and 300 mM, between 75 mM and 200 mM, between 75 mM and 175 mM, between 75 mM and 150 mM, between 100 mM and 300 mM, between 100 mM and 200 mM, between 100 mM and 175 mM, or between 100 mM and 150 mM sodium chloride. In a further embodiment, a formulation of the invention comprises 10 mM. 25 mM, 50 mM, 75 mM, 80 mM, 100 mM, 125 mM, 150 mM, 175 mM, 200 mM, or 300 mM sodium chloride.

In one embodiment, a formulation of the invention comprises an amino acid. In one embodiment, a formulation of the invention comprises an amino acid salt. In one embodiment, a formulation of the invention comprises an amino acid selected from the group consisting of lysine, arginine, and histidine. In one embodiment, a formulation of the invention comprises at least about 25 mM of an amino acid, at least about 50 mM of an amino acid, at least about 100 mM of an amino acid, at least about 150 mM of an amino acid, at least about 200 mM of an amino acid, at least about 250 mM of an amino acid, at least about 300 mM of an amino acid, at least about 350 mM of an amino acid, or at least about 400 mM of an amino acid. In another embodiment, a formulation of the invention comprises between about 25 mM and about 250 mM, between about 25 mM and about 300 mM, between about 25 mM and about 350 mM, between about 25 mM and about 400 mM, between about 50 mM and about 250 mM, between about 50 mM and about 300 mM, between about 50 mM and about 350 mM, between about 50 mM and about 400 mM, between about 100 mM and about 250 mM, between about 100 mM and about 300 mM, between about 100 mM and about 400 mM, between about 150 mM and about 250 mM, between about 150 mM and about 300 mM, or between about 150 mM and about 400 mM of an amino acid. In a further embodiment, a formulation of the invention comprises about 25 mM, about 50 mM, about 100 mM, about 150 mM, about 200 mM, about 250 mM, about 300 mM, about 350 mM, or about 400 mM of an amino acid. In a specific embodiment, a formulation of the invention comprises about 25 mM of an amino acid. In a specific embodiment, a formulation of the invention comprises about 50 mM of an amino acid. In a specific embodiment, a formulation of the invention comprises about 75 mM of an amino acid. In a specific embodiment, a formulation of the invention comprises about 100 mM of an amino acid. In a specific embodiment, a formulation of the invention comprises about 200 mM of an amino acid.

In one embodiment, a formulation of the invention comprises trehalose and an amino acid. In one embodiment, a formulation of the invention comprises trehalose and an amino acid at a molar ratio of about 0.1, about 0.5, about 0.75, about 1, about 5, about 10, about 20, about 30, about 40, about 50, about 60, about 70, about 80, about 90, about 100, about 200, or about 300, In one embodiment, a formulation of the invention comprises trehalose and an amino acid at a molar ratio of about 1.5, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, or about 4. In a specific embodiment, a formulation of the invention comprises trehalose and an amino acid at a molar ratio of about 2.1. In a specific embodiment, a formulation of the invention comprises trehalose and an amino acid at a molar ratio of about 2.2. In a specific embodiment, a formulation of the invention comprises trehalose and an amino acid at a molar ratio of about 2.4. In a specific embodiment, a formulation of the invention comprises trehalose and an amino acid at a molar ratio of about 2.5. In a specific embodiment, a formulation of the invention comprises trehalose and an amino acid at a molar ratio of about 2.6. In a specific embodiment, a formulation of the invention comprises trehalose and an amino acid at a molar ratio of about 2.7.

The formulations of the invention may further comprise a surfactant. The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Pharmaceutically acceptable surfactants like polysorbates (e.g. polysorbates 20 or 80); polyoxamers (e.g. poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e g lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g. Pluronics, PF68 etc), can optionally be added to the formulations of the invention to reduce aggregation. Surfactants are particularly useful if a pump or plastic container is used to administer the formulation. The presence of a pharmaceutically acceptable surfactant mitigates the propensity for the protein to aggregate. In a specific embodiment, the formulations of the invention comprise a polysorbate which is at a concentration ranging from between about 0.001% to about 1%, or about 0.001% to about 0.1%, or about 0.01% to about 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysothate is polysorbate-80. In a specific embodiment, the formulations of the invention comprise a polysorbate which is at a concentration ranging from between 0.001% to 1%, or 0.001% to 0.1%, or 0.01% to 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysothate is polysorbate-80.

In one embodiment, a formulation of the invention comprises a surfactant. In one embodiment, a formulation of the invention comprises Polysorbate 20, Polysorbate 40, Polysothate 60, or Polysothate 80. In a specific embodiment, a formulation of the invention comprises Polysorbate 80.

In one embodiment, a formulation of the invention comprises at least about 0.001%, at least about 0.002%, at least about 0.005%, at least about 0.01%, at least about 0.02%, at least about 0.05%, at least about 0.1%, at least about 0.2%, or at least about 0.5% Polysothate 80. In another embodiment, a formulation of the invention comprises between about 0.001% and about 0.5%, between about 0.001% and about 0.2%, between about 0.001% and about 0.1%, between about 0.001% and about 0.05%, between about 0.002% and about 0.5%, between about 0.002% and about 0.2%, between about 0.002% and about 0.1%, between about 0.002% and about 0.05%, between about 0.005% and about 0.5%, between about 0.005% and about 0.2%, between about 0.005% and about 0.1%, between about 0.005% and about 0.05%, between about 0.01% and about 0.5%, between about 0.01% and about 0.2%, between about 0.01% and about 0.1%, or between about 0.01% and about 0.05% Polysorbate 80. In a further embodiment, a formulation of the invention comprises about 0.001%, about 0.002%, about 0.005%, about 0.01%, about 0.02%, about 0.05%, about 0.1%, about 0.2%, and about 0.5% Polysorbate 80. In a specific embodiment, a formulation of the invention comprises about 0.02% Polysorbate 80. In a specific embodiment, a formulation of the invention comprises about 0.04% Polysorbate 80. In a specific embodiment, a formulation of the invention comprises about 0.05% Polysorbate 80.

In one embodiment, a formulation of the invention comprises at least 0.001%, at least 0.002%, at least 0.005%, at least 0.01%, at least 0.02%, at least 0.05%, at least 0.1%, at least 0.2%, or at least 0.5% Polysorbate 80. In another embodiment, a formulation of the invention comprises between 0.001% and 0.5%, between 0.001% and 0.2%, between 0.001% and 0.1%, between 0.001% and 0.05%, between 0.002% and 0.5%, between 0.002% and 0.2%, between 0.002% and 0.1%, between 0.002% and 0.05%, between 0.005% and 0.5%, between 0.005% and 0.2%, between 0.005% and 0.1%, between 0.005% and 0.05%, between 0.01% and 0.5%, between 0.01% and 0.2%, between 0.01% and 0.1%, or between 0.01% and 0.05% Polysorbate 80. In a further embodiment, a formulation of the invention comprises 0.001%, 0.002%, 0.005%, 0.01%, 0.02%, 0.05%, 0.1%, 0.2%, and 0.5% Polysorbate 80. In a specific embodiment, a formulation of the invention comprises 0.02% Polysorbate 80. In a specific embodiment, a formulation of the invention comprises 0.04% Polysorbate 80. In a specific embodiment, a formulation of the invention comprises 0.05% Polysorbate 80.

Optionally, the formulations of the invention may further comprise other common excipients and/or additives including, but not limited to, diluents, binders, stabilizers, lipophilic solvents, preservatives, adjuvants, or the like. Pharmaceutically acceptable excipients and/or additives may be used in the formulations of the invention. Commonly used excipients/additives, such as pharmaceutically acceptable chelators (for example, but not limited to, EDTA, DTPA or EGTA) can optionally be added to the formulations of the invention to reduce aggregation. These additives are particularly useful if a pump or plastic container is used to administer the formulation.

Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (for example, but not limited to, hexahydrate), alkylparaben (methyl, ethyl, propyl, butyl and the like), benzalkonium chloride, benzethonium chloride, sodium dehydroacetate and thimerosal, or mixtures thereof can optionally be added to the formulations of the invention at any suitable concentration such as between about 0.001% to about 5%, or any range or value therein. The concentration of preservative used in the formulations of the invention is a concentration sufficient to yield a microbial effect. Such concentrations are dependent on the preservative selected and are readily determined by the skilled artisan.

Other contemplated excipients/additives, which may be utilized in the formulations of the invention include, for example, flavoring agents, antimicrobial agents, sweeteners, antioxidants, antistatic agents, lipids such as phospholipids or fatty acids, steroids such as cholesterol, protein excipients such as serum albumin (human serum albumin (HSA), recombinant human albumin (rHA)), gelatin, casein, salt-forming counterions such as sodium and the like. These and additional known pharmaceutical excipients and/or additives suitable for use in the formulations of the invention are known in the art, e.g., as listed in “Remington: The Science & Practice of Pharmacy”, 21st ed., Lippincott Williams & Wilkins, (2005), and in the “Physician's Desk Reference”, 60th ed., Medical Economics, Montvale, N.J. (2005). Pharmaceutically acceptable carriers can be routinely selected that are suitable for the mode of administration, solubility and/or stability of Fc variant protein as well known in the art or as described herein.

It will be understood by one skilled in the art that the formulations of the invention may be isotonic with human blood, that is the formulations of the invention have essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonity of the formulation. Tonicity modifiers suitable for this invention include, but are not limited to, saccharides, salts and amino acids.

In certain embodiments, the formulations of the present invention have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm.

In certain embodiments, the formulations of the present invention have an osmotic pressure from 100 mOSm to 1200 mOSm, or from 200 mOSm to 1000 mOSm, or from 200 mOSm to 800 mOSm, or from 200 mOSm to 600 mOSm, or from 250 mOSm to 500 mOSm, or from 250 mOSm to 400 mOSm, or from 250 mOSm to 350 mOSm.

Concentration of any one or any combination of various components of the formulations of the invention are adjusted to achieve the desired tonicity of the final formulation. For example, the ratio of the carbohydrate excipient to antibody may be adjusted according to methods known in the art (e.g., U.S. Pat. No. 6,685,940). In certain embodiments, the molar ratio of the carbohydrate excipient to antibody may be from about 100 moles to about 1000 moles of carbohydrate excipient to about 1 mole of antibody, or from about 200 moles to about 6000 moles of carbohydrate excipient to about 1 mole of antibody, or from about 100 moles to about 510 moles of carbohydrate excipient to about 1 mole of antibody, or from about 100 moles to about 600 moles of carbohydrate excipient to about 1 mole of antibody.

Concentration of any one or any combination of various components of the formulations of the invention are adjusted to achieve the desired tonicity of the final formulation. For example, the ratio of the carbohydrate excipient to antibody may be adjusted according to methods known in the art (e.g., U.S. Pat. No. 6,685,940). In certain embodiments, the molar ratio of the carbohydrate excipient to antibody may be from 100 moles to 1000 moles of carbohydrate excipient to 1 mole of antibody, or from 200 moles to 6000 moles of carbohydrate excipient to 1 mole of antibody, or from 100 moles to 510 moles of carbohydrate excipient to 1 mole of antibody, or from 100 moles to 600 moles of carbohydrate excipient to 1 mole of antibody.

The desired isotonicity of the final formulation may also be achieved by adjusting the salt concentration of the formulations. Salts that are pharmaceutically acceptable and suitable for this invention as tonicity modifiers include, but are not limited to, sodium chloride, sodium succinate, sodium sulfate, potassium chloride, magnesium chloride, magnesium sulfate, and calcium chloride. In specific embodiments, formulations of the inventions comprise NaCl, MgCl₂, and/or CaCl₂. In one embodiment, concentration of NaCl is between about 75 mM and about 150 mM. In another embodiment, concentration of MgCl₂ is between about 1 mM and about 100 mM. Amino acids that are pharmaceutically acceptable and suitable for this invention as tonicity modifiers include, but are not limited to, proline, alanine, L-arginine, asparagine, L-aspartic acid, glycine, serine, lysine, and histidine.

In one embodiment the formulations of the invention are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with antibodies, even trace amounts of harmful and dangerous endotoxin must be removed. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the invention should be sterile. The formulations of the invention may be sterilized by various sterilization methods, including sterile filtration, radiation, etc. In one embodiment, the antibody formulation is filter-sterilized with a presterilized 0.22-micron filter. Sterile compositions for injection can be formulated according to conventional pharmaceutical practice as described in “Remington: The Science & Practice of Pharmacy”, 21st ed., Lippincott Williams & Wilkins, (2005). Formulations comprising antibodies, such as those disclosed herein, ordinarily will be stored in lyophilized form or in solution. It is contemplated that sterile compositions comprising antibodies are placed into a container having a sterile access port, for example, an intravenous solution bag or vial having an adapter that allows retrieval of the formulation, such as a stopper pierceable by a hypodermic injection needle. In one embodiment, a composition of the invention is provided as a pre-filled syringe.

In one embodiment, a formulation of the invention is a lyophilized formulation. The term “lyophilized” or “freeze-dried” includes a state of a substance that has been subjected to a drying procedure such as lyophilization, where at least 50% of moisture has been removed.

The phrase “bulking agent” includes a compound that is pharmaceutically acceptable and that adds bulk to a lyo cake. Bulking agents known to the art include, for example, carbohydrates, including simple sugars such as dextrose, ribose, fructose and the like, alcohol sugars such as mannitol, inositol and sorbitol, disaccharides including trehalose, sucrose and lactose, naturally occurring polymers such as starch, dextrans, chitosan, hyaluronate, proteins (e.g., gelatin and serum albumin), glycogen, and synthetic monomers and polymers.

A “lyoprotectant” is a molecule which, when combined with a protein of interest, significantly prevents or reduces chemical and/or physical instability of the protein upon lyophilization and subsequent storage. Lyoprotectants include, but are not limited to, sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; Pluronics™; and combinations thereof. Additional examples of lyoprotectants include, but are not limited to, glycerin and gelatin, and the sugars mellibiose, melezitose, raffinose, mannotriose and stachyose. Examples of reducing sugars include, but are not limited to, glucose, maltose, lactose, maltulose, iso-maltulose and lactulose. Examples of non-reducing sugars include, but are not limited to, non-reducing glycosides of polyhydroxy compounds selected from sugar alcohols and other straight chain polyalcohols. Examples of sugar alcohols include, but are not limited to, monoglycosides, compounds obtained by reduction of disaccharides such as lactose, maltose, lactulose and maltulose. The glycosidic side group can be either glucosidic or galactosidic. Additional examples of sugar alcohols include, but are not limited to, glucitol, maltitol, lactitol and iso-maltulose. In specific embodiments, trehalose or sucrose is used as a lyoprotectant.

The lyoprotectant is added to the pre-lyophilized formulation in a “lyoprotecting amount” which means that, following lyophilization of the protein in the presence of the lyoprotecting amount of the lyoprotectant, the protein essentially retains its physical and chemical stability and integrity upon lyophilization and storage.

In one embodiment, the molar ratio of a lyoprotectant (e.g., trehalose) and anti-IL-6 antibody molecules of a formulation of the invention is at least about 10, at least about 50, at least about 100, at least about 200, or at least about 300. In another embodiment, the molar ratio of a lyoprotectant (e.g., trehalose) and anti-IL-6 antibody molecules of a formulation of the invention is about 1, is about 2, is about 5, is about 10, about 50, about 100, about 200, or about 300.

A “reconstituted” formulation is one which has been prepared by dissolving a lyophilized antibody formulation in a diluent such that the antibody is dispersed in the reconstituted formulation. The reconstituted formulation is suitable for administration (e.g. parenteral administration) to a patient to be treated with the protein of interest and, in certain embodiments of the invention, may be one which is suitable for intravenous administration.

The “diluent” of interest herein is one which is pharmaceutically acceptable (safe and non-toxic for administration to a human) and is useful for the preparation of a liquid formulation, such as a formulation reconstituted after lyophilization. In some embodiments, diluents include, but are not limited to, sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

In one embodiment, a formulation of the invention is a lyophilized formulation comprising an IL-6 antibody of the invention, wherein at least about 90%, at least about 95%, at least about 97%, at least about 98%, or at least about 99% of said antibody may be recovered from a vial upon shaking said vial for 4 hours at a speed of 400 shakes per minute wherein said vial is filled to half of its volume with said formulation. In another embodiment, a formulation of the invention is a lyophilized formulation comprising an IL-6 antibody of the invention, wherein at least about 90%, at least about 95%, at least about 97%, at least about 98%, or at least about 99% of said antibody may be recovered from a vial upon subjecting the formulation to three freeze/thaw cycles wherein said vial is filled to half of its volume with said formulation. In a further embodiment, a formulation of the invention is a lyophilized formulation comprising an IL-6 antibody of the invention, wherein at least about 90%, at least about 95%, at least about 97%, at least about 98%, or at least about 99% of said antibody may be recovered by reconstituting a lyophilized cake generated from said formulation.

In one embodiment, a formulation of the invention is a lyophilized formulation comprising an IL-6 antibody of the invention, wherein at least about 90%, at least about 95%, at least about 97%, at least about 98%, or at least about 99% of said antibody may be recovered from a vial upon shaking said vial for 4 hours at a speed of 400 shakes per minute wherein said vial is filled to half of its volume with said formulation. In another embodiment, a formulation of the invention is a lyophilized formulation comprising an IL-6 antibody of the invention, wherein at least about 90%, at least about 95%, at least about 97%, at least about 98%, or at least about 99% of said antibody may be recovered from a vial upon subjecting the formulation to three freeze/thaw cycles wherein said vial is filled to half of its volume with said formulation. In a further embodiment, a formulation of the invention is a lyophilized formulation comprising an IL-6 antibody of the invention, wherein at least about 90%, at least about 95%, at least about 97%, at least about 98%, or at least about 99% of said antibody may be recovered by reconstituting a lyophilized cake generated from said formulation.

In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 40° C. for at least about 1 week, at least about 2 weeks, at least about 3 weeks, at least about 4 weeks, at least about 5 weeks, or at least about 6 weeks. In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 40° C. for at least about 1 month, at least about 2 months, at least about 3 months, at least about 4 months, at least about 5 months, or at least about 6 months.

In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 5° C. for at least about 1 year, at least about 2 years, at least about 3 years, at least about 4 years, or at least about 5 years.

In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 40° C. for about 1 week, about 2 weeks, about 3 weeks, about 4 weeks, about 5 weeks, or about 6 weeks. In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 40° C. for about 1 month, about 2 months, about 3 months, about 4 months, about 5 months, or about 6 months.

In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a lyophilized formulation of the invention comprises anti-IL-6 antibody molecules of the invention, wherein at least about 90%, at least about 95%, at least about 97%, at least about 98%, or at least about 99% of said antibody is recovered by reconstituting said lyophilized formulation upon storage at about 5° C. for about 1 year, about 2 years, about 3 years, about 4 years, or about 5 years.

In one embodiment, a formulation of the invention is a reconstituted formulation. In certain embodiments, a reconstituted liquid formulation of the invention is prepared from a lyophilized formulation described herein.

In one embodiment, a reconstituted liquid formulation of the invention comprises an anti-IL-6 antibody of the invention at the same concentration as the pre-lyophilized liquid formulation.

In one embodiment, a reconstituted liquid formulation of the invention comprises an anti-IL-6 antibody of the invention at a higher concentration than the pre-lyophilized liquid formulation. In specific embodiments, a reconstituted liquid formulation of the invention comprises about 2 fold, about 3 fold, about 4 fold, about 5 fold, about 6 fold, about 7 fold, about 8 fold, about 9 fold, about 10 fold, about 15 fold, about 20 fold, about 30 fold, about 40 fold higher concentration of an anti-IL-6 antibody of the invention than the pre-lyophilized liquid formulation.

In one embodiment, a reconstituted liquid formulation of the invention comprises an anti-IL-6 antibody of the invention at a lower concentration than the pre-lyophilized liquid formulation. In specific embodiments, a reconstituted liquid formulation of the invention comprises about 2 fold, about 3 fold, about 4 fold, about 5 fold, about 6 fold, about 7 fold, about 8 fold, about 9 fold, about 10 fold, about 15 fold, about 20 fold, about 30 fold, about 40 fold lower concentration of an anti-IL-6 antibody of the invention than the pre-lyophilized liquid formulation.

In one embodiment, a reconstituted liquid formulation of the invention is an aqueous formulation. In a specific embodiment, a reconstituted liquid formulation of the invention is an aqueous formulation wherein the aqueous carrier is distilled water.

In one embodiment, a reconstituted formulation of the invention is sterile.

In one embodiment, a reconstituted formulation of the invention is homogeneous.

In one embodiment, a reconstituted formulation of the invention is isotonic. In one embodiment, a reconstituted formulation of the invention is hypotonic. In one embodiment, a reconstituted formulation of the invention is hypertonic.

In certain embodiments, reconstituted formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, reconstituted formulations of the invention contain no detectable particles greater than 40 μm, or greater than 30 μm.

In certain embodiments, after storage for about 1 hour, about 2 hours, about 3 hours, about 4 hours, about 5 hours, about 6 hours, about 7 hours, about 8 hours, about 9 hours, about 12 hours, about 15 hours, about 18 hours, or about 24 hours reconstituted liquid formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 am, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, liquid formulations of the invention contain no detectable particles greater than 40 μm, or greater than 30 μm.

In specific embodiments, the pharmaceutical compositions include, but are not limited to:

(a) a sterile liquid formulation consisting of 100 mg/ml of antibody, 25 mM histidine, 1.6 mM glycine at pH 6.0;
(b) a sterile liquid formulation consisting of 100 mg/ml of antibody and 25 mM histidine at pH 6.0;
(c) a sterile liquid formulation consisting of 5 mg/ml antibody, 20 mM Citric acid, 100 mM NACl, 1.5% mannitol, 50 □l DTPA, and 0.02% PS80 at pH 6.0;
   (d) a sterile liquid formulation consisting of 100 mg/ml of antibody, 25 mM histidine, 8% trehalose, and 0.02% PS80 at pH 6.0;
   (e) a sterile liquid formulation consisting of 20 mg/ml of antibody, 10 mM His, 2.35% (w/v) Lysine-HCl, and 0.02% PS-80 (w/v) at pH 6.0;
   (f) a sterile liquid formulation consisting of 5 mg/ml of antibody, 10 mM Sodium citrate buffer, NaCl (0.15M) and Tween 80 (0.02%) at pH 6.0;
   (g) a sterile liquid formulation consisting of 100 mg/ml of antibody, 10 mM histidine and 150 mM NaCl at pH 6.0.

In one embodiment, a formulation of the invention stabilizes an anti-IL-6 antibody of the invention. In one embodiment, a formulation of the invention prevents aggregation of an anti-IL-6 antibody of the invention. In another embodiment, a formulation of the invention prevents fragmentation of an anti-IL-6 antibody of the invention.

In one embodiment, a formulation of the invention is stable upon storage at about 40° C. for at least about 1 week, at least about 2 weeks, at least about 3 weeks, or at least about 4 weeks. In one embodiment, a formulation of the invention is stable upon storage at about 40° C. for at least about 1 month, at least about 2 months, at least about 3 months, at least about 4 months, at least about 5 months, or at least about 6 months. In a specific embodiment, a formulation of the invention is stable upon storage in a pre-filled syringe.

In one embodiment, a formulation of the invention is stable upon storage at about 25° C. for at least about 1 week, at least about 2 weeks, at least about 3 weeks, or at least about 4 weeks. In one embodiment, a formulation of the invention is stable upon storage at about 25° C. for at least about 1 month, at least about 2 months, at least about 3 months, at least about 4 months, at least about 5 months, or at least about 6 months. In a specific embodiment, a formulation of the invention is stable upon storage in a pre-filled syringe.

In one embodiment, a formulation of the invention is stable upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention is stable upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stable upon storage in a pre-filled syringe.

In one embodiment, a formulation of the invention is stable upon storage at about 40° C. for about 1 week, about 2 weeks, about 3 weeks, or about 4 weeks. In one embodiment, a formulation of the invention is stable upon storage at about 40° C. for about 1 month, about 2 months, about 3 months, about 4 months, about 5 months, or about 6 months. In a specific embodiment, a formulation of the invention is stable upon storage in a pre-filled syringe.

In one embodiment, a formulation of the invention is stable upon storage at about 25° C. for about 1 week, about 2 weeks, about 3 weeks, or about 4 weeks. In one embodiment, a formulation of the invention is stable upon storage at about 25° C. for about 1 month, about 2 months, about 3 months, about 4 months, about 5 months, or about 6 months. In a specific embodiment, a formulation of the invention is stable upon storage in a pre-filled syringe.

In one embodiment, a formulation of the invention is stable upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention is stable upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stable upon storage in a pre-filled syringe.

The present inventions provide stable formulations comprising anti-IL-6 antibodies of the invention. The stability of said antibody can be assessed by degrees of aggregation, degradation or fragmentation, as measured by HPSEC, reverse phase chromatography, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation comprising a reference antibody. For example, a reference formulation may be a reference standard frozen at −70° C. consisting of 10 mg/ml of a reference antibody antibody (including antibody fragment thereof) (for example, but not limited to, an antibody comprising the 16C4 variable region and an Fc region having complex N-glycoside-linked sugar chains in which fucose is not bound to N-acetylglucosamine in the reducing end in the sugar chain) in 10 mM histidine (pH 6.0) that contains 75 mMNaCl and 4% trehalose, which reference formulation regularly gives a single monomer peak (e.g., ≧95% area) by HPSEC. In certain embodiments, a reference formulation is identical to the formulation whose stability is tested; the reference formulation may be stored frozen at −70° C. during the stability testing to preserve the reference formulation in its original condition. For example, the reference standard for assessing any loss of IL-6 antigen binding activity in a formulation stored at 40° C. may be the identical formulation stored at −70° C. for 30 days. The overall stability of a formulation comprising an antibody (including antibody fragment thereof) may also be assessed by various immunological assays including, for example, ELISA and radioimmunoassay using isolated antigen molecules. Furthermore, the stability of a formulation comprising an antibody may also be assessed using various assays designed to measure a functional characteristic of the antibody, for example, assays designed to measure antigen binding affinity, in vitro ADCC activity, in vivo depletion activity, in vitro CDC activity, inhibition assays, cell proliferation assays, etc.

In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 40° C. for at least about 1 week, at least about 2 weeks, at least about 3 weeks, or at least about 4 weeks. In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 25° C. for at least about 1 week, at least about 2 weeks, at least about 3 weeks, or at least about 4 weeks. In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention comprises an anti-IL-6 antibody of the invention having an extended in vivo half life.

In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 40° C. for about 1 week, about 2 weeks, about 3 weeks, or about 4 weeks. In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 40° C. for about 1 month, about 2 months, about 3 months, about 4 months, about 5 months, or about 6 months. In a specific embodiment, a formulation of the invention comprises an anti-IL-6 antibody of the invention having an extended in vivo half life.

In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 25° C. for about 1 week, about 2 weeks, about 3 weeks, or about 4 weeks. In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 25° C. for about 1 month, about 2 months, about 3 months, about 4 months, about 5 months, or about 6 months. In a specific embodiment, a formulation of the invention comprises an anti-IL-6 antibody of the invention having an extended in vivo half life.

In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-IL-6 antibody of the invention that has an IL-6 binding activity that is at least 50%, at least 60%, at least 70%, at least 80%, at least 90%, at least 95%, or at least 99% of the IL-6 binding activity of a reference antibody, wherein said formulation was stored at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 40° C. for at least about 1 week, at least about 2 weeks, at least about 3 weeks, or at least about 4 weeks. In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life. As used herein, the terms “at most’ and “no more than” have the same meaning.

In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 40° C. for at least about 1 week, at least about 2 weeks, at least about 3 weeks, or at least about 4 weeks. In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 25° C. for at least about 1 week, at least about 2 weeks, at least about 3 weeks, or at least about 4 weeks. In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention comprises an anti-IL-6 antibody of the invention having an extended in vivo half life.

In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 40° C. for about 1 week, about 2 weeks, about 3 weeks, or about 4 weeks. In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 40° C. for about 1 month, about 2 months, about 3 months, about 4 months, about 5 months, or about 6 months. In a specific embodiment, a formulation of the invention comprises an anti-IL-6 antibody of the invention having an extended in vivo half life.

In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 25° C. for about 1 week, about 2 weeks, about 3 weeks, or about 4 weeks. In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 25° C. for about 1 month, about 2 months, about 3 months, about 4 months, about 5 months, or about 6 months. In a specific embodiment, a formulation of the invention comprises an anti-IL-6 antibody of the invention having an extended in vivo half life.

In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-IL-6 antibody of the invention, wherein the antibody loses at most 50%, at most 40%, at most 30%, at most 20%, at most 10%, at most 5%, or at most 1% of its IL-6 binding activity during storage of the formulation at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 25° C. for at least about 1 week, at least about 2 weeks, at least about 3 weeks, or at least about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 40° C. for about 1 week, about 2 weeks, about 3 weeks, or about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 25° C. for about 1 week, about 2 weeks, about 3 weeks, or about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 25° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-IL-6 antibody of the invention, wherein less than 1%, less than 2%, less than 3%, less than 4%, less than 5%, less than 7% or less than 10% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 40° C. for at least about 1 week, at least about 2 weeks, at least about 3 weeks, or at least about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 25° C. for at least about 1 week, at least about 2 weeks, at least about 3 weeks, or at least about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 40° C. for about 1 week, about 2 weeks, about 3 weeks, or about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 40° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 25° C. for about 1 week, about 2 weeks, about 3 weeks, or about 4 weeks. In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 25° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-IL-6 antibody of the invention, wherein less than 1%, less than 2%, less than 3%, less than 4%, less than 5%, less than 7% or less than 10% of said antibody is fragmented as determined by RP-HPLC or SEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention is clear and colorless as determined by visual inspection upon storage at about 40° C. for at least about 1 week, at least about 2 weeks, at least about 3 weeks, or at least about 4 weeks. In one embodiment, a formulation of the invention is clear and colorless as determined by visual inspection upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention is clear and colorless as determined by visual inspection upon storage at about 25° C. for at least about 1 week, at least about 2 weeks, at least about 3 weeks, or at least about 4 weeks. In one embodiment, a formulation of the invention is clear and colorless as determined by visual inspection upon storage at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention is clear and colorless as determined by visual inspection upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention is clear and colorless as determined by visual inspection upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention is clear and colorless as determined by visual inspection upon storage at about 40° C. for about 1 week, about 2 weeks, about 3 weeks, or about 4 weeks. In one embodiment, a formulation of the invention is clear and colorless as determined by visual inspection upon storage at about 40° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention is clear and colorless as determined by visual inspection upon storage at about 25° C. for about 1 week, about 2 weeks, about 3 weeks, or about 4 weeks. In one embodiment, a formulation of the invention is clear and colorless as determined by visual inspection upon storage at about 25° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention is clear and colorless as determined by visual inspection upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention is clear and colorless as determined by visual inspection upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-IL-6 antibody of the invention having an extended in vivo half life.

In certain embodiments, the formulations of the invention maintain improved aggregation profiles upon storage, for example, for extended periods (for example, but not limited to 1 week, 1 month, 6 months, 1 year, 2 years, 3 years or 5 years) at room temperature or 4° C. or for periods (such as, but not limited to 1 week, 2 weeks, 3 weeks, 1 month, 2 months, 3 months, or 6 months) at elevated temperatures such as 38° C.-42° C. In certain embodiments, the formulations maintain improved aggregation profiles upon storage while exposed to light or stored in the dark in a variety of humidity conditions including but not limited to a relative humidity of up to 10%, or up to 20%, or up to 30%, or up to 40%, or up to 50%, or up to 60%, or up to 70%, or up to 80%, or up to 90%, or up to 100%. It will be understood in the art that the term “ambient” conditions generally refers to temperatures of about 20° C. at a relative humidity of between 10% and 60% with exposure to light. Similarly, temperatures between about 2° C. and about 8° C. at a relative humidity of less then about 10% are collectively referred to as “4° C.” or “5° C.”, temperatures between about 23° C. and about 27° C. at a relative humidity of about 60% are collectively referred to as “25° C.” and temperatures between about 38° C. and about 42° C. at a relative humidity of about 75% are collectively referred to as “40° C.” In a specific embodiment, a formulation of the invention is stored in a pre-filled syringe.

In certain embodiments, after storage at 4° C. for at least one month, the formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, the formulations of the invention contain no detectable particles greater than 40 μm, or greater than 30 μm. In a specific embodiment, a formulation of the invention is stored in a pre-filled syringe.

Numerous methods useful for determining the degree of aggregation, and/or types and/or sizes of aggregates present in a protein formulation (e.g., antibody formulation of the invention) are known in the art, including but not limited to, size exclusion chromatography (SEC), high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques. For example, size exclusion chromatography (SEC) may be performed to separate molecules on the basis of their size, by passing the molecules over a column packed with the appropriate resin, the larger molecules (e.g. aggregates) will elute before smaller molecules (e.g. monomers). The molecules are generally detected by UV absorbance at 280 nm and may be collected for further characterization. High pressure liquid chromatographic columns are often utilized for SEC analysis (HP-SEC). Specific SEC methods are detailed in the section entitled “Examples” infra. Alternatively, analytical ultracentrifugation (AUC) may be utilized. AUC is an orthogonal technique which determines the sedimentation coefficients (reported in Svedberg, S) of macromolecules in a liquid sample. Like SEC, AUC is capable of separating and detecting antibody fragments/aggregates from monomers and is further able to provide information on molecular mass. Protein aggregation in the formulations may also be characterized by particle counter analysis using a coulter counter or by turbidity measurements using a turbidimeter. Turbidity is a measure of the amount by which the particles in a solution scatter light and, thus, may be used as a general indicator of protein aggregation. In addition, non-reducing polyacrylamide gel electrophoresis (PAGE) or capillary gel electrophoresis (CGE) may be used to characterize the aggregation and/or fragmentation state of antibodies or a fragment thereof in a formulation of the invention.

In one embodiment, a formulation of the invention is for parenteral administration. In one embodiment, a formulation of the invention is an injectable formulation. In specific embodiments, the formulation of the invention is suitable for intravenous, intramuscular, intraarterial, intrathecal, intracapsular, intraorbital, intracardiac, intradermal, intraperitoneal, perineural, transtracheal, subcutaneous, subcuticular, intraarticular, subcapsular, subarachnoid, intraspinal, epidural and infrasternal injection and infusion. In one embodiment, a formulation of the invention is for intravenous, subcutaneous, or intramuscular administration. In a specific embodiment, a formulation of the invention comprises an anti-IL-6 antibody of the invention wherein said formulation is for subcutaneous injection. In a specific embodiment, a formulation of the invention comprises an anti-IL-6 antibody of the invention wherein said formulation is for intravenous injection. In a specific embodiment, a formulation of the invention is stored in a pre-filled syringe. In a specific embodiment, a formulation of the invention comprises an anti-IL-6 antibody of the invention having an extended in vivo half life.

In one embodiment, a formulation of the invention is for intravenous administration wherein said formulation comprises between about 1 mg/ml and about 60 mg/ml, between about 1 mg/ml and about 50 mg/ml, between about 1 mg/ml and about 40 mg/ml, between about 10 mg/ml and about 60 mg/ml, between about 10 mg/ml and about 50 mg/ml, between about 10 mg/ml and about 40 mg/ml, between about 20 mg/ml and about 60 mg/ml, between about 20 mg/ml and about 50 mg/ml, between about 20 mg/ml and about 40 mg/ml, between about 30 mg/ml and about 60 mg/ml, between about 30 mg/ml and about 50 mg/ml, or between about 30 mg/ml and about 40 mg/ml of an anti-IL-6 antibody of the invention of the invention. In a specific embodiment, a formulation of the invention comprises an anti-IL-6 antibody of the invention having an extended in vivo half life.

In one embodiment, a formulation of the invention is for perineural or intrathecal administration wherein the formulation comprises between about 0.01 μg/ml and about 50 μg/ml, between about 0.05 μg/ml and about 45 mg/ml, between about 0.1 μg/ml and about 30 μg/ml, between about 0.15 μg/ml and about 25 μg/ml, between about 0.2 μg/ml and about 20 μg/ml, between about 0.25 μg/ml and about 17.5 μg/ml, between about 0.5 μg/ml and about 15 μg/ml, between about 0.75 μg/ml and about 12.5 μg/ml, between about 0.6 μg/ml and about 10 μg/ml, between about 1.0 μg/ml and about 8 μg/ml, between about 1.25 μg/ml and about 7.5 μg/ml, or between about 1.5 μg/ml and about 6 μg/ml of an anti-IL-6 antibody of the invention of the invention. In a specific embodiment, a formulation of the invention comprises an anti-IL-6 antibody of the invention having an extended in vivo half life.

In one embodiment, a formulation of the invention is for subcutaneous administration wherein said formulation comprises between about 1 mg/ml and about 100 mg/ml, between about 1 mg/ml and about 150 mg/ml, between about 1 mg/ml and about 200 mg/ml, between about 25 mg/ml and about 100 mg/ml, between about 25 mg/ml and about 150 mg/ml, between about 25 mg/ml and about 200 mg/ml, between about 50 mg/ml and about 100 mg/ml, between about 50 mg/ml and about 150 mg/ml, between about 50 mg/ml and about 200 mg/ml, between about 75 mg/ml and about 100 mg/ml, between about 75 mg/ml and about 150 mg/ml or between about 75 mg/ml and about 200 mg/ml of an anti-IL-6 antibody of the invention of the invention. In a specific embodiment, a formulation of the invention is provided in a pre-filled syringe. In a specific embodiment, a formulation of the invention comprises an anti-IL-6 antibody of the invention having an extended in vivo half life.

In one embodiment, a formulation of the invention is for aerosol administration.

The present invention also provides a pharmaceutical unit dosage form suitable for parenteral administration to a human which comprises an anti-IL-6 antibody of the invention formulation in a suitable container. In one embodiment, a pharmaceutical unit dosage of the invention comprises an intravenously, subcutaneously, or intramuscularly delivered anti-IL-6 antibody of the invention formulation. In another embodiment, a pharmaceutical unit dosage of the invention comprises aerosol delivered anti-IL-6 antibody of the invention formulation. In a specific embodiment, a pharmaceutical unit dosage of the invention comprises a subcutaneously delivered anti-IL-6 antibody of the invention formulation. In another embodiment, a pharmaceutical unit dosage of the invention comprises an aerosol delivered anti-IL-6 antibody of the invention formulation. In a further embodiment, a pharmaceutical unit dosage of the invention comprises an intranasally administered anti-IL-6 antibody of the invention formulation. In one embodiment, a suitable container is a pre-filled syringe. In a specific embodiment, a formulation of the invention comprises an anti-IL-6 antibody of the invention having an extended in vivo half life.

In one embodiment, a formulation of the invention is provided in a sealed container. In a specific embodiment, a formulation of the invention is provided in a pre-filled syringe. In a specific embodiment, a formulation of the invention comprises an anti-IL-6 antibody of the invention having an extended in vivo half life.

The present invention further provided a kit comprising an anti-IL-6 antibody of the invention formulation of the invention. The invention provides a pharmaceutical pack or kit comprising one or more containers filled with a liquid formulation or lyophilized formulation of the invention. In one embodiment, a container filled with a liquid formulation of the invention is a pre-filled syringe. In a specific embodiment, the formulations of the invention comprise antibodies (including antibody fragments thereof) recombinantly fused or chemically conjugated to another moiety, including but not limited to, a heterologous protein, a heterologous polypeptide, a heterologous peptide, a large molecule, a small molecule, a marker sequence, a diagnostic or detectable agent, a therapeutic moiety, a drug moiety, a radioactive metal ion, a second antibody, and a solid support. In a specific embodiment, the formulations of the invention are formulated in single dose vials as a sterile liquid. The formulations of the invention may be supplied in 3 cc USP Type I borosilicate amber vials (West Pharmaceutical Services—Part No. 6800-0675) with a target volume of 1.2 mL. Optionally associated with such container(s) can be a notice in the form prescribed by a governmental agency regulating the manufacture, use or sale of pharmaceuticals or biological products, which notice reflects approval by the agency of manufacture, use or sale for human administration. In another embodiment, a formulation of the invention may be supplied in a pre-filled syringe.

In one embodiment, a container filled with a liquid formulation of the invention is a pre-filled syringe. Any pre-filled syringe known to one of skill in the art may be used in combination with a liquid formulation of the invention. Pre-filled syringes that may be used are described in, for example, but not limited to, PCT Publications WO05032627, WO08094984, WO9945985, WO03077976, U.S. Pat. No. 6,792,743, U.S. Pat. No. 5,607,400, U.S. Pat. No. 5,893,842, U.S. Pat. No. 7,081,107, U.S. Pat. No. 7,041,087, U.S. Pat. No. 5,989,227, U.S. Pat. No. 6,807,797, U.S. Pat. No. 6,142,976, U.S. Pat. No. 5,899,889, US Patent Publications US20070161961A1, US20050075611A1, US20070092487A1, US20040267194A1, US20060129108A1. Pre-filled syringes may be made of various materials. In one embodiment a pre-filled syringe is a glass syringe. In another embodiment a pre-filled syringe is a plastic syringe. One of skill in the art understands that the nature and/or quality of the materials used for manufacturing the syringe may influence the stability of a protein formulation stored in the syringe. For example, it is understood that silicon based lubricants deposited on the inside surface of the syringe chamber may affect particle formation in the protein formulation. In one embodiment, a pre-filled syringe comprises a silicone based lubricant. In one embodiment, a pre-filled syringe comprises baked on silicone. In another embodiment, a pre-filled syringe is free from silicone based lubricants. One of skill in the art also understands that small amounts of contaminating elements leaching into the formulation from the syringe barrel, syringe tip cap, plunger or stopper may also influence stability of the formulation. For example, it is understood that tungsten introduced during the manufacturing process may adversely affect formulation stability. In one embodiment, a pre-filled syringe may comprise tungsten at a level above 500 ppb. In another embodiment, a pre-filled syringe is a low tungsten syringe. In another embodiment, a pre-filled syringe may comprise tungsten at a level between about 500 ppb and about 10 ppb, between about 400 ppb and about 10 ppb, between about 300 ppb and about 10 ppb, between about 200 ppb and about 10 ppb, between about 100 ppb and about 10 ppb, between about 50 ppb and about 10 ppb, between about 25 ppb and about 10 ppb.

Articles of Manufacture

The present invention also encompasses a finished packaged and labeled pharmaceutical product. This article of manufacture includes the appropriate unit dosage form in an appropriate vessel or container such as a glass vial, pre-filled syringe or other container that is hermetically sealed. In one embodiment, the unit dosage form is provided as a sterile particulate free solution comprising an anti-IL-6 antibody that is suitable for parenteral administration. In another embodiment, the unit dosage form is provided as a sterile lyophilized powder comprising an anti-IL-6 antibody that is suitable for reconstitution.

In one embodiment, the unit dosage form is suitable for intravenous, intramuscular, intranasal, oral, topical or subcutaneous delivery. Thus, the invention encompasses sterile solutions suitable for each delivery route. The invention further encompasses sterile lyophilized powders that are suitable for reconstitution.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, and other monitoring information.

Specifically, the invention provides an article of manufacture comprising packaging material, such as a box, bottle, tube, vial, container, pre-filled syringe, sprayer, insufflator, intravenous (i.v.) bag, envelope and the like; and at least one unit dosage form of a pharmaceutical agent contained within said packaging material, wherein said pharmaceutical agent comprises a liquid formulation containing an antibody. The packaging material includes instruction means which indicate how that said antibody can be used to prevent, treat and/or manage one or more symptoms associated with a disease or disorder.

Pharmaceutical compositions for oral administration, such as for example single domain antibody molecules (e.g. “Nanobodies™”) etc are also envisaged in the present invention. Such oral formulations may be in tablet, capsule, powder, liquid or semi-solid form. A tablet may comprise a solid carrier, such as gelatin or an adjuvant. Liquid pharmaceutical compositions generally comprise a liquid carrier, such as water, petroleum, animal or vegetable oils, mineral oil or synthetic oil. Physiological saline solution, dextrose or other saccharide solution or glycols, such as ethylene glycol, propylene glycol or polyethylene glycol may be included.

For intra-venous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

Binding members of the present invention may be formulated in liquid, semi-solid or solid forms depending on the physicochemical properties of the molecule and the route of delivery. Formulations may include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example. Formulations of binding members will depend upon the intended route of delivery: for example, formulations for pulmonary delivery may consist of particles with physical properties that ensure penetration into the deep lung upon inhalation; topical formulations (e.g. for treatment of scarring, e.g. dermal scarring) may include viscosity modifying agents, which prolong the time that the drug is resident at the site of action. A binding member may be prepared with a carrier that will protect the binding member against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known to those skilled in the art (Robinson, J. R. ed., (1978) Sustained and Controlled Release Drug Delivery Systems, Marcel Dekker, Inc., New York).

Treatment may be given orally (such as for example single domain antibody molecules (e.g. “Nanobodies™”)) by injection (for example, subcutaneously, intra-articular, intra-venously, intra-peritoneal, intra-arterial or intra-muscularly), by inhalation, intra-tracheal, by the intra-vesicular route (instillation into the urinary bladder), or topically (for example intra-ocular, intra-nasal, rectal, into wounds, on skin). The treatment may be administered by pulse infusion, particularly with declining doses of the binding member. The route of administration can be determined by the physicochemical characteristics of the treatment, by special considerations for the disease or by the requirement to optimize efficacy or to minimize side-effects. One particular route of administration is intra-venous. Another route of administering pharmaceutical compositions of the present invention is subcutaneously. It is envisaged that treatment will not be restricted to use in the clinic. Therefore, subcutaneous injection using a needle-free device is also advantageous.

A composition may be administered alone or in combination with other treatments, either simultaneously or sequentially dependent upon the condition to be treated.

A binding member of the invention may be used as part of a combination therapy in conjunction with an additional medicinal component. Combination treatments may be used to provide significant synergistic effects, particularly the combination of a binding member of the invention with one or more other drugs. A binding member of the invention may be administered concurrently or sequentially or as a combined preparation with another therapeutic agent or agents, for the treatment of one or more of the conditions listed herein.

A binding member of the invention may be used as a chemosensitiser whereby it can increase therapeutic efficacy of cytotoxic agents, and may thus be provided for administration in combination with one or more cytotoxic agents, either simultaneously or sequentially. The binding member may also be used as a radio sensitiser whereby it can improve efficacy of radiation, and may thus be provided for administration in combination with radiation, either simultaneously or sequentially.

A binding member according to the present invention may be provided in combination or addition with one or more of the following agents:
a cytokine or agonist or antagonist of cytokine function (e.g. an agent which acts on cytokine signalling pathways, such as a modulator of the SOCS system), such as an alpha-, beta- and/or gamma-interferon; insulin-like growth factor type I (IGF-1), its receptors and associated binding proteins; interleukins (IL), e.g. one or more of IL-1 to -33, and/or an interleukin antagonist or inhibitor, such as anakinra; inhibitors of receptors of interleukin family members or inhibitors of specific subunits of such receptors, a tumor necrosis factor alpha (TNF-α) inhibitor, such as an anti-TNF monoclonal antibodies (for example infliximab, adalimumab and/or CDP-870) and/or a TNF receptor antagonist, e.g. an immunoglobulin molecule (such as etanercept) and/or a low-molecular-weight agent, such as pentoxyfylline;
a modulator of B cells, e.g. a monoclonal antibody targeting B-lymphocytes (such as CD20 (rituximab) or MRA-aIL16R) or T-lymphocytes (e.g. CTLA4-Ig, HuMax Il-15 or Abatacept);
a modulator that inhibits osteoclast activity, for example an antibody to RANKL;
   a modulator of chemokine or chemokine receptor function, such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 and CXCR6 (for the C-X-C family) and CX₃CR1 for the C-X₃-C family;
   an inhibitor of matrix metalloproteases (MMPs), i.e. one or more of the stromelysins, the collagenases and the gelatinases as well as aggrecanase, especially collagenase-1 (MMP-1), collagenase-2 (MMP-8), collagenase-3 (MMP-13), stromelysin-1 (MMP-3), stromelysin-2 (MMP-10) and/or stromelysin-3 (MMP-11) and/or MMP-9 and/or MMP-12, e.g. an agent such as doxycycline;
   a leukotriene biosynthesis inhibitor, 5-lipoxygenase (5-LO) inhibitor or 5-lipoxygenase activating protein (FLAP) antagonist, such as zileuton; ABT-761; fenleuton; tepoxalin; Abbott-79175; Abbott-85761; N-(5-substituted)-thiophene-2-alkylsulfonamides; 2,6-di-tert-butylphenolhydrazones; methoxytetrahydropyrans such as Zeneca ZD-2138; the compound SB-210661; a pyridinyl-substituted 2-cyanonaphthalene compound, such as L-739,010; a 2-cyanoquinoline compound, such as L-746,530; indole and/or a quinoline compound, such as MK-591, MK-886 and/or BAY×1005;
   a receptor antagonist for leukotrienes (LT) B4, LTC4, LTD4, and LTE4, selected from the group consisting of the phenothiazin-3-1 s, such as L-651,392; amidino compounds, such as CGS-25019c; benzoxalamines, such as ontazolast; benzenecarboximidamides, such as BIIL 284/260; and compounds, such as zafirlukast, ablukast, montelukast, pranlukast, verlukast (1VIK-679), RG-12525, Ro-245913, iralukast (CGP 45715A) and BAY×7195;
   a phosphodiesterase (PDE) inhibitor, such as a methylxanthanine, e g theophylline and/or aminophylline; and/or a selective PDE isoenzyme inhibitor, e.g. a PDE4 inhibitor and/or inhibitor of the isoform PDE4D and/or an inhibitor of PDE5;
   a histamine type 1 receptor antagonist, such as cetirizine, loratadine, desloratadine, fexofenadine, acrivastine, terfenadine, astemizole, azelastine, levocabastine, chlorpheniramine, promethazine, cyclizine, and/or mizolastine (generally applied orally, topically or parenterally);
   a proton pump inhibitor (such as omeprazole) or gastroprotective histamine type 2 receptor antagonist;
   an antagonist of the histamine type 4 receptor;
   an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride and ethylnorepinephrine hydrochloride;
   an anticholinergic agent, e.g. a muscarinic receptor (M1, M2, and M3) antagonist, such as atropine, hyoscine, glycopyrrrolate, ipratropium bromide, tiotropium bromide, oxitropium bromide, pirenzepine and telenzepine;
   a beta-adrenoceptor agonist (including beta receptor subtypes 1-4), such as isoprenaline, salbutamol, formoterol, salmeterol, terbutaline, orciprenaline, bitolterol mesylate and/or pirbuterol, e.g. a chiral enantiomer thereof;
   a chromone, e.g. sodium cromoglycate and/or nedocromil sodium;
   a glucocorticoid, such as flunisolide, triamcinolone acetonide, beclomethasone dipropionate, budesonide, fluticasone propionate, ciclesonide, and/or mometasone furoate;
   an agent that modulate nuclear hormone receptors, such as a PPAR;
   an immunoglobulin (Ig) or Ig preparation or an antagonist or antibody modulating Ig function, such as anti-IgE (e g omalizumab);
   other systemic or topically-applied anti-inflammatory agent, e.g. thalidomide or a derivative thereof, a retinoid, dithranol and/or calcipotriol;
   combinations of aminosalicylates and sulfapyridine, such as sulfasalazine, mesalazine, balsalazide, and olsalazine; and immunomodulatory agents, such as the thiopurines; and corticosteroids, such as budesonide;
   an antibacterial agent, e.g. a penicillin derivative, a tetracycline, a macrolide, a beta-lactam, a fluoroquinolone, metronidazole and/or an inhaled aminoglycoside; and/or an antiviral agent, e.g. acyclovir, famciclovir, valaciclovir, ganciclovir, cidofovir; amantadine, rimantadine; ribavirin; zanamavir and/or oseltamavir; a protease inhibitor, such as indinavir, nelfinavir, ritonavir and/or saquinavir; a nucleoside reverse transcriptase inhibitor, such as didanosine, lamivudine, stavudine, zalcitabine, zidovudine; a non-nucleoside reverse transcriptase inhibitor, such as nevirapine, efavirenz;
   a cardiovascular agent, such as a calcium channel blocker, beta-adrenoceptor blocker, angiotensin-converting enzyme (ACE) inhibitor, angiotensin-2 receptor antagonist; lipid lowering agent, such as a statin and/or librate; a modulator of blood cell morphology, such as pentoxyfylline; a thrombolytic and/or an anticoagulant, e.g. a platelet aggregation inhibitor;
   a CNS agent, such as an antidepressant (such as sertraline), anti-Parkinsonian drug (such as deprenyl, L-dopa, ropinirole, pramipexole; MAOB inhibitor, such as selegine and rasagiline; comP inhibitor, such as tasmar; A-2 inhibitor, dopamine reuptake inhibitor, NMDA antagonist, nicotine agonist, dopamine agonist and/or inhibitor of neuronal nitric oxide synthase) and an anti-Alzheimer's drug, such as donepezil, rivastigmine, tacrine, COX-2 inhibitor, propentofylline or metrifonate;
   an agent for the treatment of acute and chronic pain, e.g. a centrally or peripherally-acting analgesic, such as an opioid analogue or derivative, carbamazepine, phenytoin, sodium valproate, amitryptiline or other antidepressant agent, paracetamol, or non-steroidal anti-inflammatory agent;
   a parenterally or topically-applied (including inhaled) local anaesthetic agent, such as lignocaine or an analogue thereof;
   an anti-osteoporosis agent, e.g. a hormonal agent, such as raloxifene, or a biphosphonate, such as alendronate;
   (i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitors including VLA-4 antagonist; (vi) a cathepsin; (vii) a kinase inhibitor, e.g. an inhibitor of tyrosine kinases (such as Btk, Itk, Jak3 MAP examples of inhibitors might include Gefitinib, Imatinib mesylate), a serine/threonine kinase (e.g. an inhibitor of MAP kinase, such as p38, INK, protein kinases A, B and C and IKK), or a kinase involved in cell cycle regulation (e.g. a cylin dependent kinase); (viii) a glucose-6 phosphate dehydrogenase inhibitor; (ix) a kinin-B₁- and/or B₂-receptor antagonist; (x) an anti-gout agent, e.g. colchicine; (xi) a xanthine oxidase inhibitor, e.g. allopurinol; (xii) a uricosuric agent, e.g. probenecid, sulfinpyrazone, and/or benzbromarone; (xiii) a growth hormone secretagogue; (xiv) transforming growth factor (TGFβ); (xv) platelet-derived growth factor (PDGF); (xvi) fibroblast growth factor, e.g. basic fibroblast growth factor (bFGF); (xvii) granulocyte macrophage colony stimulating factor (GM-CSF); (xviii) capsaicin cream; (xix) a tachykinin NK₁ and/or NK₃ receptor antagonist, such as NKP-608C, SB-233412 (talnetant) and/or D-4418; (xx) an elastase inhibitor, e.g. UT-77 and/or ZD-0892; (xxi) a TNF-alpha converting enzyme inhibitor (TACE); (xxii) induced nitric oxide synthase (iNOS) inhibitor or (xxiii) a chemoattractant receptor-homologous molecule expressed on TH2 cells (such as a CRTH2 antagonist); (xxiv) an inhibitor of a P38 (xxv) agent modulating the function of Toll-like receptors (TLR) and (xxvi) an agent modulating the activity of purinergic receptors, such as P2X7; (xxvii) an inhibitor of transcription factor activation, such as NFkB, API, and/or STATS.

An inhibitor may be specific or may be a mixed inhibitor, e.g. an inhibitor targeting more than one of the molecules (e.g. receptors) or molecular classes mentioned above.

The binding member could also be used in association with a chemotherapeutic agent or another tyrosine kinase inhibitor in co-administration or in the form of an immunoconjugate. Fragments of said antibody could also be use in bispecific antibodies obtained by recombinant mechanisms or biochemical coupling and then associating the specificity of the above described antibody with the specificity of other antibodies able to recognize other molecules involved in the activity for which IL-6 is associated.

For treatment of an inflammatory disease, a binding member of the invention may be combined with one or more agents, such as non-steroidal anti-inflammatory agents (hereinafter NSAIDs) including non-selective cyclo-oxygenase (COX)-1/COX-2 inhibitors whether applied topically or systemically, such as piroxicam, diclofenac, propionic acids, such as naproxen, flurbiprofen, fenoprofen, ketoprofen and ibuprofen, fenamates, such as mefenamic acid, indomethacin, sulindac, azapropazone, pyrazolones, such as phenylbutazone, salicylates, such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intra-muscular, intra-venous or intra-articular routes); methotrexate, leflunomide; hydroxychloroquine, d-penicillamine, auranofin or other parenteral or oral gold preparations; analgesics; diacerein; intra-articular therapies, such as hyaluronic acid derivatives; and nutritional supplements, such as glucosamine.

A binding member of the invention can also be used in combination with an existing therapeutic agent for the treatment of cancer. Suitable agents to be used in combination include:

(i) antiproliferative/antineoplastic drugs and combinations thereof, as used in medical oncology, such as Gleevec (imatinib mesylate), alkylating agents (for example cis-platin, carboplatin, cyclophosphamide, nitrogen mustard, melphalan, chlorambucil, busulphan and nitrosoureas); antimetabolites (for example antifolates, such as fluoropyrimidines like 5-fluorouracil and tegafur, raltitrexed, methotrexate, cytosine arabinoside, hydroxyurea, gemcitabine and paclitaxel); antitumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecins);
(ii) cytostatic agents, such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase, such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors, such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents, such as those which inhibit the effects of vascular endothelial growth factor (for example the anti-vascular endothelial cell growth factor antibody bevacizumab, compounds, such as those disclosed in International Patent Applications WO 97/22596, WO 97/30035, WO 97/32856 and WO 98/13354, each of which is incorporated herein in its entirety) and compounds that work by other mechanisms (for example linomide, inhibitors of integrin αvβ3 function and angiostatin);
(vi) vascular damaging agents, such as combretastatin A4 and compounds disclosed in International Patent Applications WO 99/02166, WO 00/40529, WO 00/41669, WO 01/92224, WO 02/04434 and WO 02/08213 (each of which is incorporated herein in its entirety);
(vii) antisense therapies, for example those which are directed to the targets listed above, such as ISIS 2503, an anti-ras antisense;
(viii) gene therapy approaches, including for example approaches to replace aberrant genes, such as aberrant p53 or aberrant BRCA1 or BRCA2, GDEPT (gene directed enzyme pro-drug therapy) approaches, such as those using cytosine deaminase, thymidine kinase or a bacterial nitroreductase enzyme and approaches to increase patient tolerance to chemotherapy or radiotherapy, such as multi-drug resistance gene therapy; and
(ix) immunotherapeutic approaches, including for example ex vivo and in vivo approaches to increase the immunogenicity of patient tumor cells, such as transfection with cytokines, such as interleukin 2, interleukin 4 or granulocyte macrophage colony stimulating factor, approaches to decrease T-cell anergy, approaches using transfected immune cells, such as cytokine-transfected dendritic cells, approaches using cytokine-transfected tumor cell lines and approaches using anti-idiotypic antibodies.

A binding member of the invention and one or more of the above additional medicinal components may be used in the manufacture of a medicament. The medicament may be for separate or combined administration to an individual, and accordingly may comprise the binding member and the additional component as a combined preparation or as separate preparations. Separate preparations may be used to facilitate separate and sequential or simultaneous administration, and allow administration of the components by different routes e.g. oral and parenteral administration.

In accordance with the present invention, compositions provided may be administered to mammals. Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the type of binding member, the method of administration, the scheduling of administration and other factors known to medical practitioners. Prescription of treatment, e.g. decisions on dosage etc, is within the responsibility of general practitioners and other medical doctors and may depend on the severity of the symptoms and/or progression of a disease being treated. Appropriate doses of antibody are well known in the art (Ledermann J. A. et al. (1991) Int. J. Cancer 47: 659-664; Bagshawe K. D. et al. (1991) Antibody, Immunoconjugates and Radiopharmaceuticals 4: 915-922). Specific dosages indicated herein or in the Physician's Desk Reference (2003) as appropriate for the type of medicament being administered may be used. A therapeutically effective amount or suitable dose of a binding member of the invention can be determined by comparing its in vitro activity and in vivo activity in an animal model. Methods for extrapolation of effective dosages in mice and other test animals to humans are known. The precise dose will depend upon a number of factors, including whether the antibody is for diagnosis, prevention or for treatment, the size and location of the area to be treated, the precise nature of the antibody (e.g. whole antibody, fragment or diabody) and the nature of any detectable label or other molecule attached to the antibody. A typical antibody dose will be in the range 100 μg to 1 g for systemic applications, and 1 μg to 1 mg for topical applications. An initial higher loading dose, followed by one or more lower doses, may be administered. Typically, the antibody will be a whole antibody, e.g. the IgG1 isotype. This is a dose for an effective treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician. Treatments may be every two to four weeks for subcutaneous administration and every four to eight weeks for intra-venous administration. Treatment may be periodic, and the period between administrations is about two weeks or more, e.g. about three weeks or more, about four weeks or more, or about once a month. Treatment may be given before, and/or after surgery, and/or may be administered or applied directly at the anatomical site of surgical treatment.

IL-6 binding members of the invention may offer advantages in terms of dosage and administration requirements, compared with antibodies to sIL-6Ra. As noted elsewhere herein, circulating levels of IL-6 are significantly lower than circulating levels of sIL-6Ra in disease. Accordingly, use of an IL-6 binding member, as opposed to an anti-IL-6R binding member, has significant advantages in that the amount of drug to be manufactured for each dose to patients may be lower. Also if the dose of an anti-IL6 therapeutic is lower there may be significant advantages in that the low dose facilitates sub-cutaneous injections as well as intra-venous (i.v.) injections. It is well known to those skilled in the art that sub-cutaneous dosing may be limited by the amount of binding member, e.g. antibody molecule, required per dose. This is due to the sub-cutaneous injections being limited by the volume that can be injected at one site in the skin. Sub-cutaneous injection volumes of 1.2 ml or less are typically utilised. As it may be increasingly difficult to formulate a binding member for sub-cutaneous injection at concentrations greater than 50 mg/ml, doses above 100 mg via this route usually require multiple injections and more discomfort for the patient.

Having a lower dose anti-IL-6 therapeutic may also require a lower “loading” dose of antibody to inhibit all the systemic IL-6 compared with the systemic sIL-6Ra as this is at higher concentrations.

Further benefits may be associated with targeting IL-6 rather than IL-6 receptor, representing additional advantages of binding members of the invention as compared with binding members for IL-6Ra.

For example, there are literature reports which show that the circulating levels of IL-6 are significantly lower than circulating levels of sIL-6Ra in disease (Desgeorges et al. (1997) J. Rheumatol 24:1510; Yokota et al. (2005) Arth & Rheum 52(3): 818-25). As the levels of sIL-6R are significantly higher than IL-6 levels, more anti-sIL-6R binding member may be required to neutralise the sIL-6Ra, compared with the amount of anti-IL-6 binding member required to neutralise IL-6. Hence, a lower dose of an anti-ligand binding member may be needed, compared with if an anti-receptor binding member were used.

Targeting IL-6 ligand rather than IL-6 receptor may reduce levels of IL-6 in disease but still allow IL-6 levels to increase during infection, where IL-6 is up-regulated as part of the immune response.

Kawano et al. (Nature (1988) 332:83) showed that IL-6 was a potent growth factor and showed that myeloma cells freshly isolated from patients produced IL-6 and express its receptors. Moreover, anti-IL-6 antibody inhibits the in vitro growth of myeloma cells. This is direct evidence that an autocrine loop is operating in oncogenesis of human myelomas. Subsequent to that study, Van Zaanen et al. (J. Clin. Invest. (1996) 98:1441-1448) demonstrated that the production of IL-6 in multiple myeloma patients decreases when treated with an anti-IL-6 ligand antibody.

A number of further studies show that IL-6 is involved in an autocrine feedback loop in other cell types e.g. smooth muscle cells (SMC) (Klouche et al., (1999) J. Immunol. 163(8) 4583-9), U373-MG astroglioma cells (Oh et al., (2001) J. Immunol. 166: 2695-704), 3T3 adipocytes (Fasshauer et al., (2003) Horm. Metab. Res. 35(3) 147-52), neurons (Mara et al., (1998) Proc. Natl. Acad. Sci USA 95(6) 3251-6), endothelial cells (Modur et al., (1997) J. Clin. Invest. 100(1) 2752-6) and Kaposi's sarcoma cells (Murakami-Morl et al., (1996) Cell Growth Differ. 7(12) 1697-703). Inhibition of IL-6 using an anti-IL6 binding member in disease can therefore lead to a decrease in the basal disease production of IL-6.

Further, anti-IL-6 binding members bind IL-6 in the systemic circulation, in contrast with binding members to IL-6 receptor which need to penetrate the tissue in order to occupy the receptor on the surface of cells involved in the pathology of the disease to be treated.

Binding members to IL-6 may form an equilibrium with IL-6 in the systemic circulation, having the effect of causing gradients across barriers e.g. the synovial membrane, which has the net effect of removing active IL-6 from the joint and forming an inactive complex with the binding member. The consequence of this is that an IL-6 binding member may have quicker onset and dosing regime may be different and potentially easier to optimise, compared with an IL-6R binding member.

IL-6 signalling is mediated by IL-6 binding to IL-6R and that complex binding to gp130. Given that IL-6 and IL-6Ra binding is of nanomolar affinity (about 5 nM) and that IL6:IL6R complex and gp130 binding is of picomolar affinity, a binding member which targets IL-6 faces a lower amount of competition for IL-6 binding and so may suppress a greater proportion of IL-6 signalling. Although this may also apply for a binding member targeting the soluble IL-6Ra and preventing IL-6:IL-6Ra complex formation, if the IL-6Ra is membrane bound then because of steric constraints it may be more difficult for an anti-IL-6Ra to bind and inhibit the IL-6Ra presented on the membrane.

The invention provides methods of prevention, treatment and/or management of a disorder, for example, a disorder associated with or characterized by aberrant expression and/or activity of IL-6, a disorder associated with aberrant expression and/or activity of IL-6 receptor, an autoimmune disorder, an inflammatory disorder, a proliferative disorder, an infection, or one or more symptoms thereof by administrating to a subject of an effective amount of compositions of the invention. Various delivery systems are known and can be used to administer a composition of the present invention or a prophylactic or therapeutic agent. Methods of administering compositions of the present invention or a therapy (e.g., a prophylactic or therapeutic agent) include, but are not limited to, parenteral administration (e.g., intradermal, intramuscular, intraperitoneal, intravenous perineural and subcutaneous), epidural administration, topical administration, and mucosal administration (for example, but not limited to, intranasal and oral routes). In a specific embodiment, compositions of the present invention are administered intramuscularly, intravenously, or subcutaneously. In one embodiment, the compositions of the invention are administered subcutaneously. The formulations may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The invention also provides that a composition of the present invention is packaged in a hermetically sealed container such as an ampoule or sachette indicating the quantity of antibody (including antibody fragment thereof). In one embodiment, a composition of the present invention is in a hermetically sealed container indicating the quantity and concentration of the antibody (including antibody fragment thereof). In one embodiment, a composition of the present invention is supplied in a hermetically sealed container and comprises about 10 mg/ml, about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, about 200 mg/ml, about 250 mg/ml, or about 300 mg/ml of an antibody (including antibody fragment thereof) that specifically binds to IL-6, in a quantity of about 1 nil, about 2 nil, about 3 nil, about 4 nil, about 5 ml, 6 about nil, about 7 nil, about 8 nil, about 9 ml, about 10 nil, about 15 nil, or about 20 nil. In a specific embodiment of the invention, a composition of the invention is supplied in a hermetically sealed container and comprises at least about 15 mg/ml, at least about 20 mg/ml, at least about 25 mg/ml, at least about 50 mg/ml, at least about 100 mg/ml, at least about 150 mg/ml, at least about 175 mg/ml, at least about 200 mg/ml, at least about 250 mg/ml or at least about 300 mg/ml of an antibody (including antibody fragment thereof) that specifically binds to IL-6 (for example, but not limited to, Antibody 18E) for intravenous injections, and at least about 15 mg/ml, at least about 20 mg/ml, at least about 50 mg/ml, at least about 80 mg/ml, at least about 100 mg/ml, at least about 150 mg/ml, at least about 175 mg/ml, at least about 200 mg/ml, at least about 250 mg/ml or at least about 300 mg/ml of an antibody that specifically binds to IL-6 (for example, but not limited to, Antibody 18E) for repeated subcutaneous administration.

The amount of a composition of the present invention which will be effective in the prevention, treatment and/or management of a disease or disorder associated with or characterized by aberrant expression and/or activity of IL-6, a disease or disorder associated with or characterized by aberrant expression and/or activity of the IL-6 receptor or one or more subunits thereof, an autoimmune disease, an autoimmune disease, transplant rejection, graft versus host disease, or one or more symptoms thereof can be determined by standard clinical techniques well-known in the art or described herein. The precise dose to be employed in the composition will also depend on the route of administration, and the seriousness of the inflammatory disorder, or autoimmune disorder, and should be decided according to the judgment of the practitioner and each patients circumstances. Effective doses may be extrapolated from dose-response curves derived from in vitro or animal model test systems.

For compositions of the antibodies encompassed by the invention, the dosage administered to a patient may be calculated using the patient's weight in kilograms (kg) multiplied by the dose to be administered in mg/kg. The required volume (in mL) to be given is then determined by taking the mg dose required divided by the concentration of the antibody formulation. The final calculated required volume will be obtained by pooling the contents of as many vials as are necessary into syringe(s) to administer the antibody formulation of the invention. The final calculated required volume will be obtained by pooling the contents of as many vials as are necessary into syringe(s) to administer the drug. A maximum volume of 2.0 mL of the antibody formulation can be injected per site. The dose (in mL) can be calculated using the following formula: Dose (mL)=[volunteer weight] (kg)×[dose] mg/kg÷100 mg/mL of the antibody formulation. Antibodies of the invention have extended half-life within the human body. Thus, lower dosages of antibodies of the invention and less frequent administration is often possible. Further, the dosage, volume and frequency of administration of compositions of the present invention may be reduced by increasing the concentration of an antibody in the compositions, increasing affinity and/or avidity of the antibody.

In a specific embodiment, the dosage administered to a patient will be calculated using the patient's weight in kilograms (kg) multiplied by the dose to be administered in mg/kg. The required volume (in mL) to be given is then determined by taking the mg dose required divided by the concentration of the antibody (including antibody fragment thereof) in the formulations (100 mg/mL). The final calculated required volume will be obtained by pooling the contents of as many vials as are necessary into syringe(s) to administer the drug. A maximum volume of 2.0 mL of antibody (including antibody fragment thereof) in the formulations can be injected per site.

In a specific embodiment, 0.1 to 20 mg/kg/week, 1 to 15 mg/kg/week, 2 to 8 mg/week, 3 to 7 mg/kg/week, or 4 to 6 mg/kg/week of an antibody (including antibody fragment thereof) that specifically binds to IL-6 (for example, but not limited to, 1Antibody 18E) in a composition of the invention is administered to a subject with an inflammatory disorder or an autoimmune disorder. In another embodiment, a subject is administered one or more doses of a prophylactically or therapeutically effective amount of a composition of the invention, wherein the prophylactically or therapeutically effective amount is not the same for each dose.

In one embodiment, a composition of the invention is administered in a dosing regimen that maintains the plasma concentration of the antibody specific for IL-6 at a desirable level (e.g., from about 0.1 to about 100 μg/ml), which continuously blocks IL-6 activity. In a specific embodiment, the plasma concentration of the antibody is maintained at about 0.2 μg/ml, about 0.5 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 15 μg/ml, about 20 μg/ml, about 25 μg/ml, about 30 μg/ml, about 35 μg/ml, about 40 μg/ml, about 45 μg/ml or about 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment and/or management of a chronic disease or disorder.

In specific embodiments, a composition of the invention comprising a conjugated antibody (including antibody fragment thereof) specific for IL-6 is administered intermittently. As used herein, “a conjugated antibody or antibody fragment” refers to an antibody (including antibody fragment thereof) that is conjugated or fused to another moiety, including but not limited to, a heterologous peptide, polypeptide, another antibody (including antibody fragment thereof), a marker sequence, a diagnostic agent, a polymer, albumin, and a solid support.

In another embodiment, a human subject is administered one or more doses of a prophylactically or therapeutically effective amount of an antibody that specifically binds to IL-6 (for example, but not limited to, Antibody 18E) in a composition of the invention, wherein the dose of a prophylactically or therapeutically effective amount of the antibody in the composition of the invention administered to said subject is increased by, e.g., about 0.01 μg/kg, about 0.02 μg/kg, about 0.04 μg/kg, about 0.05 μg/kg, about 0.06 μg/kg, about 0.08 μg/kg, about 0.1 μg/kg, about 0.2 μg/kg, about 0.25 μg/kg, about 0.5 μg/kg, about 0.75 μg/kg, about 1 μg/kg, about 1.5 μg/kg, about 2 μg/kg, about 4 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, or about 125 μg/kg, as treatment progresses.

In another embodiment, a subject (e.g., a human) is administered one or more doses of a prophylactically or therapeutically effective amount of an antibody that specifically binds to IL-6 (for example, but not limited to, Antibody 18E) in a composition of the invention, wherein the dose of a prophylactically or therapeutically effective amount of the antibody in the composition of the invention administered to said subject is decreased by, e.g., about 0.01 μg/kg, about 0.02 μg/kg, about 0.04 μg/kg, about 0.05 μg/kg, about 0.06 μg/kg, about 0.08 μg/kg, about 0.1 μg/kg, about 0.2 μg/kg, about 0.25 μg/kg, about 0.5 μg/kg, about 0.75 μg/kg, about 1 μg/kg, about 1.5 μg/kg, about 2 μg/kg, about 4 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, or about 125 μg/kg, as treatment progresses

### Antibody Half-Life

In certain embodiments, the half-life of an anti-IL-6 antibody of compositions and methods of the invention is at least about 10 days. In certain embodiments, the mean half-life of an anti-IL-6 antibody of compositions and methods of the invention is at least about 20 to 40 days, 25 to 40 days, 26 to 40 days, 20 to 30 days, 25 to 30 days, 26 to 30 days, or 26 to 29 days. In still further embodiments the half-life of an anti-ICOS antibody of compositions and methods of the invention can be up to about 50 days. In certain embodiments, the half-lives of antibodies of compositions and methods of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375; and International Publication Nos. WO 98/23289 and WO 97/3461.

The serum circulation of anti-IL-6 antibodies in vivo may also be prolonged by attaching inert polymer molecules such as high molecular weight polyethyleneglycol (PEG) to the antibodies with or without a multifunctional linker either through site-specific conjugation of the PEG to the N- or C-terminus of the antibodies or via epsilon-amino groups present on lysyl residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

Further, the antibodies of compositions and methods of the invention can be conjugated to albumin in order to make the antibody more stable in vivo or have a longer half-life in vivo. The techniques are well known in the art, see, e.g., International Publication Nos. WO 93/15199, WO 93/15200, and WO 01/77137; and European Patent No. EP 413, 622, all of which are incorporated herein by reference.

Additionally, variant Fc regions that confer increased in vivo half-life on antibodies has been described (see, US Patent Publication No: US2003/0190311 A1). The use of Fc variants with extended in vivo half-life in combination with the compositions and methods of the current invention is contemplated. In one embodiment, an anti-IL-6 antibody of the invention comprises a variant Fc region with increased in vivo half-life. In a further embodiment, an anti-IL-6 antibody of the invention comprises a variant Fc region comprising at least one substitution of an amino acid residue selected from the group consisting of: residue 252, 254, and 256, wherein the amino acid residue positions are determined according to the EU convention. In a specific embodiment, an anti-IL-6 antibody of the invention comprises a variant Fc region comprising at least one amino acid substitution selected from the group consisting of: M252Y, S254T, and T256E; wherein the amino acid residue positions are determined according to the EU convention. In a further embodiment, an anti-IL-6 antibody of the invention comprises a variant Fc region comprising at least one amino acid residue selected from the group consisting of: Y at position 252, T at position 254, and E at position 256; wherein the amino acid residue positions are determined according to the EU convention.

### Fc Variants

The present invention provides anti-IL-6 antibodies comprising a variant Fc region. That is, a non naturally occurring Fc region, for example an Fc region comprising one or more non naturally occurring amino acid residues. Also encompassed by the variant Fc regions of present invention are Fc regions which comprise amino acid deletions, additions and/or modifications.

It will be understood that Fc region as used herein includes the polypeptides comprising the constant region of an antibody excluding the first constant region immunoglobulin domain Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region including, but not limited to, proteins comprising variant Fc regions, which are non naturally occurring variants of an Fc. Note: Polymorphisms have been observed at a number of Fc positions, including but not limited to Kabat 270, 272, 312, 315, 356, and 358, and thus slight differences between the presented sequence and sequences in the prior art may exist.

The present invention encompasses Fc variant proteins which have altered binding properties for an Fc ligand (e.g., an Fc receptor, C1q) relative to a comparable molecule (e.g., a protein having the same amino acid sequence except having a wild type Fc region). Examples of binding properties include but are not limited to, binding specificity, equilibrium dissociation constant (K_{D}), dissociation and association rates (k_{off} and kₒₙ respectively), binding affinity and/or avidity. It is generally understood that a binding molecule (e.g., a Fc variant protein such as an antibody) with a low K_{D} may be preferable to a binding molecule with a high K_{D}. However, in some instances the value of the kₒₙ or k_{off} may be more relevant than the value of the K_{D}. One skilled in the art can determine which kinetic parameter is most important for a given antibody application.

The affinities and binding properties of an Fc domain for its ligand may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immunoabsorbent assay (ELISA), or radioimmunoassay (RIA)), or kinetics (e.g., BIACORE® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In one embodiment, the Fc variant protein has enhanced binding to one or more Fc ligand relative to a comparable molecule. In another embodiment, the Fc variant protein has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. In a specific embodiment, the Fc variant protein has enhanced binding to an Fc receptor. In a specific embodiment, the Fc variant protein has enhanced binding to the Fc receptor FcRn.

The serum half-life of proteins comprising Fc regions may be increased by increasing the binding affinity of the Fc region for FcRn. In one embodiment, the Fc variant protein has enhanced serum half life relative to comparable molecule.

In one embodiment, the present invention provides compositions, wherein the Fc region comprises a non naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 332, 333, 334, 339, 341, 343, 370, 373, 378, 392, 416, 419, 421, 440 and 443 as numbered by the EU index as set forth in Kabat. Optionally, the Fc region may comprise a non naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In a specific embodiment, the present invention provides an Fc variant protein composition, wherein the Fc region comprises at least one non naturally occurring amino acid residue selected from the group consisting of 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 305I, 313F, 316D, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In another embodiment, the present invention provides an Fc variant protein composition, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat and at least one non naturally occurring amino acid at one or more positions are selected from the group consisting of 252Y, 254T and 256E, as numbered by the EU index as set forth in Kabat.

In one embodiment, the Fc variants of the present invention may be combined with other known Fc variants such as those disclosed in Ghetie et al., 1997, Nat Biotech. 15:637-40; Duncan et al, 1988, Nature 332:563-564; Lund et al., 1991, J. Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351. Also encompassed by the present invention are Fc regions which comprise deletions, additions and/or modifications. Still other modifications/substitutions/additions/deletions of the Fc domain will be readily apparent to one skilled in the art.

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492 (1985)), PCR mutagenesis (Higuchi, in “PCR Protocols: A Guide to Methods and Applications”, Academic Press, San Diego, pp. 177-183 (1990)), and cassette mutagenesis (Wells et al., Gene 34:315-323 (1985)). Preferably, site-directed mutagenesis is performed by the overlap-extension PCR method (Higuchi, in “PCR Technology: Principles and Applications for DNA Amplification”, Stockton Press, New York, pp. 61-70 (1989)). The technique of overlap-extension PCR (Higuchi, ibid.) can also be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351).

In some embodiments, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1. Anti-IL-6 Antibody Isolation

A detailed description of the isolation of Antibody 18 and other anti-IL-6 antibodies that may be used to practice the inventions described herein is provided in PCT Publication No. WO 2008/065378. Briefly, a precursor to Antibody 18 was isolated through a phage display library screen using recombinant human IL-6 as a target. The precursor was subjected to affinity optimization to generate several high affinity human anti-IL-6 antibodies. The characterization of these antibodies is described in PCT Publication No. WO 2008/065378. Antibody 18 is capable of blocking IL-6 binding to IL-6R. Antibody 18 binds to human and cynomolgus IL-6, but does not bind to IL-6 derived from murine, rat or dog. Antibody 18 binds to human IL-6 with an affinity that is higher than the 10 pM detection level of the BIAcore assay. The affinity of Antibody 18 to human IL-6 was estimated at 0.40 pM (95% CI 0.12 pM-0.69 pM) using the TF-1 Cell Proliferation Assay.

### Example 2. Anti-IL-6 Antibody with Increased Half-Life

### 2.1 Generation of Variant Anti-IL-6 IgG1 Antibody Comprising an Fc Region Having the M252Y, S254T and T256E Substitutions

The expression vector encoding Antibody 18 was modified using standard laboratory methods to introduce the M252Y, S254T and T256E substitutions into the Fc region. The modified Antibody 18 comprising the M252Y, S254T and T256E substitutions is hereinafter referred to as Antibody 18E or 18E.

The polynucleotides encoding the heavy and light chains of an anti-IL6 antibody may be subjected to nucleic acid sequence optimization. The final goal of the sequence optimization process is to create a coding region that is transcribed and translated at the highest possible efficiency. Sequence optimization is achieved by a combination of: (i) codon usage optimization, (ii) G/C content adaptation, (iii) elimination of internal splicing sites and premature polyadenylation sites, (iv) disruption of stable RNA secondary structures, (v) elimination of direct repeat sequences, (vi) elimination of sequences that may form stable dsRNA with host cell transcripts, (vii) eliminate sequences targeted by host cell micro RNAs, and (viii) introduction of RNA stabilizing and RNA translocation signals. Detailed sequence optimization methods are described in WO2004059556A2, WO2006015789A2, Bradel-Tretheway et al., J. Virol. Methods 111:145-56 (2003), Valencik & McDonald, Transgenic Res. 3:269-75 (2001). Alternatively, a sequence may be optimized by a commercial provider (e.g., GENEART Inc.).

Nucleotide sequences encoding the VH, VL, heavy chain and light chain of Antibody 18E were optimized following the methods described herein. The optimized nucleotide sequences encoding the VH, V1, heavy chain and light chain of 18E are disclosed as SEQ ID NOs:11-14, respectively.

Antibody 18E was expressed in a pool of CHO-K1 cells stably transfected with an expression vector comprising the coding region of the full length 18E antibody. Antibody 18E was purified from the supernatant using standard laboratory techniques.

### 2.2 In Vitro Characterization of a Variant Anti-IL-6 IgG1 Antibody Comprising an Fc Region Having the M252Y, S254T and T256E Substitutions

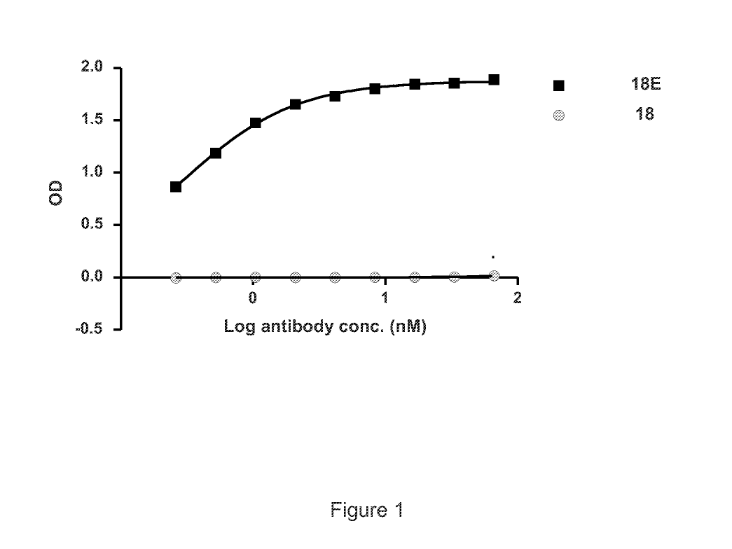

Antibody 18E comprises an Fc region having the M252Y, S254T and T256E substitutions. The presence of the M252Y, S254T and T256E substitutions in the Fc region of Antibody 18E were confirmed using an ELISA assay. The assay utilized as capture reagents two monoclonal antibodies that specifically bind to an Fc polypeptide comprising the M252Y, S254T and T256E substitutions but not to the corresponding wild type Fc polypeptide. ELISA assays were performed according to standard protocols. The ELISA titration curve obtained with one of the substitution specific monoclonal antibodies is shown in FIG. 1. Antibody 18E, but not antibody 18 was captured in an ELISA assay by an antibody specific for the M252Y, S254T and T256E Fc region substitutions. Therefore, antibody 18 comprises an Fc region having the M252Y, S254T and T256E substitutions.

Fc polypeptides comprising the M252Y, S254T and T256E substitutions have an increased binding affinity at pH 6 to FcRn compared to that of the wild type Fc polypeptide. The FcRn binding affinity of purified Antibody 18 and Antibody 18E were determined using a BIAcore assay. The assay was performed following standard protocols. Antibody 18E binds both human and cyno FcRn at pH 6 with a significantly higher affinity than that of antibody 18. Kd values as determined by BIAcore are shown in Table 1

TABLE 1
FcRn binding affinity of Antibody 18 and 18E.

| Antibody | KD huFcRn (nM) | KD cynoFcRn (nM) |
|---|---|---|
| 18 | 2610 | 1160 |
| 18E | 226 | 365 |

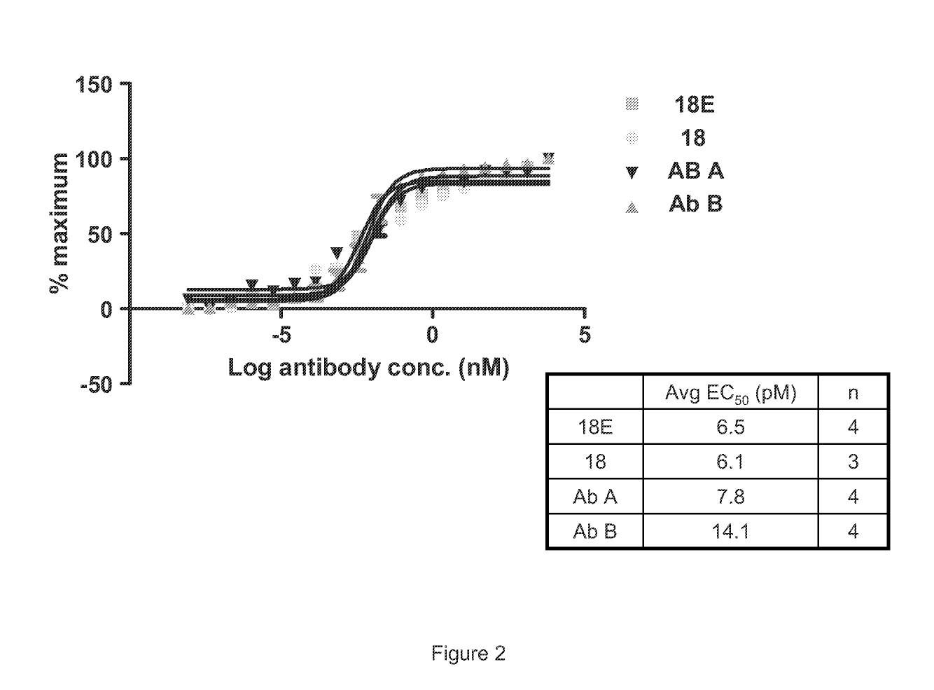

Antibody 18 and 18E bind to IL-6 with substantially equal affinity. IL-6 binding affinity of Antibody 18 and 18E were ascertained by ELISA assays. An E. coli expressed recombinant human IL-6 preparation (rhuIL-6) was used as a capture reagent. ELISA assays were performed according to standard protocols. In addition to Antibody 18 and 18E, two competitor anti-IL-6 antibodies (AB A and AB B) were also included in the assay as positive controls. An example of the data obtained is shown in FIG. 2. The EC₅₀ value for antibody 18 and 18E were 6.1 pM and 6.5 pM, respectively.

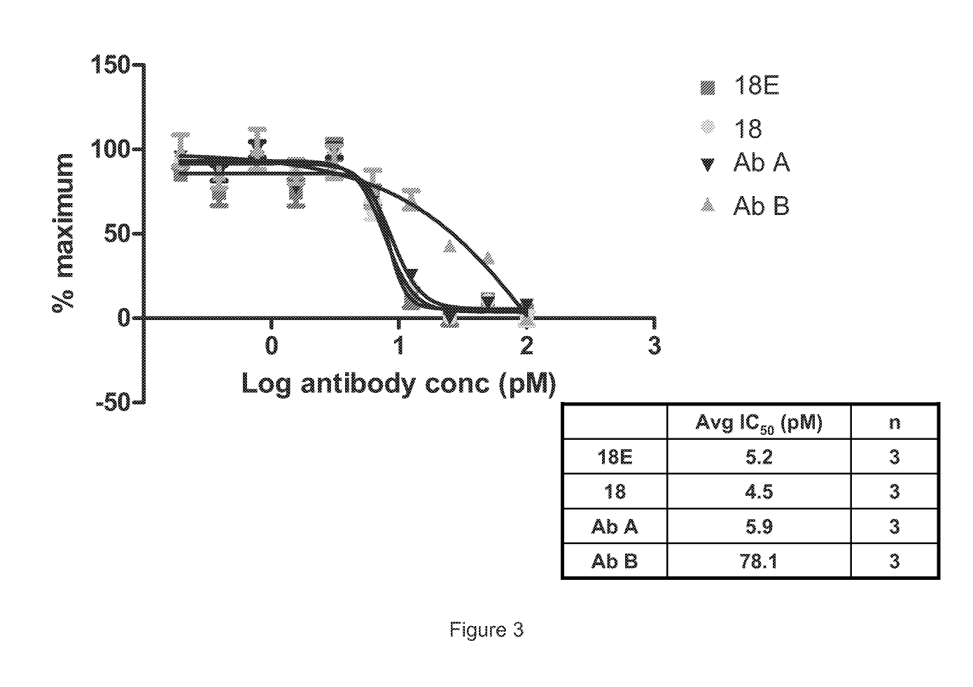

Antibody 18 and 18E inhibit IL-6 induced TF-1 cell proliferation with substantially identical efficacy. IL-6 induced TF-1 cell proliferation assay was performed substantially as described herein. In addition to Antibody 18 and 18E, two competitor anti-IL-6 antibodies (AB A and AB B) were also included in the assay as positive controls. Representative data is shown in FIG. 3. The IC₅₀ calculated for Antibody 18 and 18E were 4.5 pM and 5.2 pM, respectively.

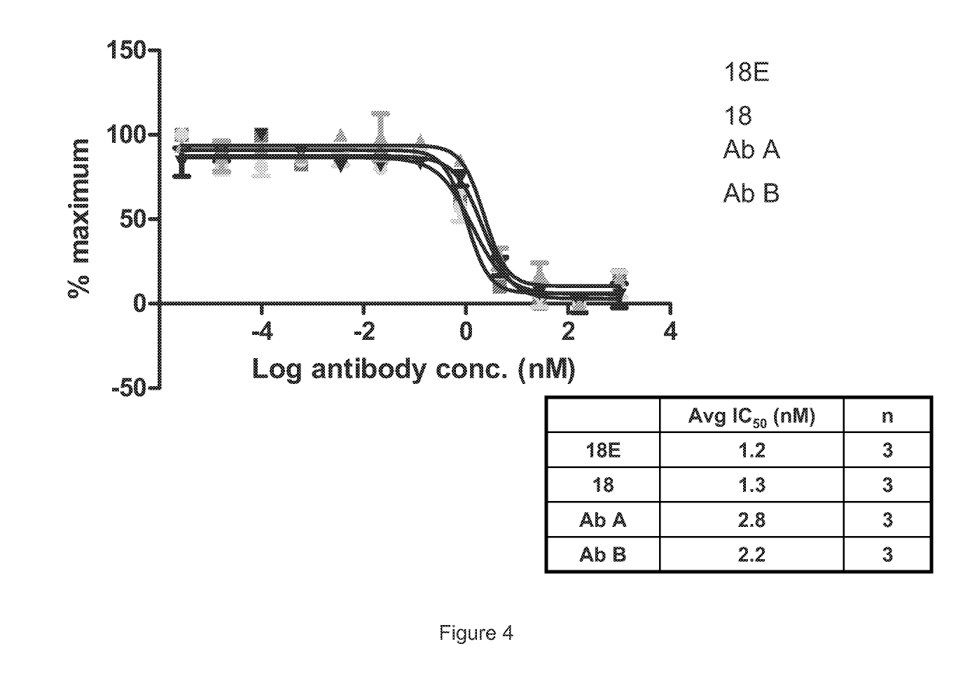

Antibody 18 and 18E inhibit endogenous IL-6 induced VEGF release from human synovial fibroblasts with substantially identical efficacy. The assay was performed substantially as described herein. In addition to Antibody 18 and 18E, two competitor anti-IL-6 antibodies (AB A and AB B) were also included in the assay as positive controls. Representative data is shown in FIG. 4. The IC₅₀ calculated for Antibody 18 and 18E were 1.3 pM and 1.2 pM, respectively.

### 2.3 In Vivo Characterization of a Variant Anti-IL-6 IgG1 Antibody Comprising an Fc Region Having the M252Y, S254T and T256E Substitutions

A single dose pharmacokinetic pharmacodynamic study in cynomolgus monkeys was performed to determine the serum half-life and clearance of Antibody 18 and 18E. The study design is outlined in Table 2.

TABLE 2
Study design for single dose pharmacokinetic experiment.

| | | Dose | | |
|---|---|---|---|---|
| Group | Treatment | (mg/kg) | Dose Route | Number |
| 1 | 18 | 5 | IV | 3 males |
| 2 | 18 | 5 | SC | 3 males |
| 3 | 18E | 5 | IV | 3 males |
| 4 | 18E | 5 | SC | 3 males |
| 5 | 18E | 50 | SC | 3 males |

IL-6 Antigen Capture PK Assay (ECL) for Quantitation of Antibody 18 or Antibody 18E in Cynomolgus Monkey Plasma: MA2400 96 well plates (MSD) were coated with 2.5 □g/ml recombinant human IL-6 (R&D Systems) overnight at 2-8° C., washed with PBS containing 0.05% Tween 20 and blocked for 1-2 hours at room temperature with I-Block Buffer (Tropix). Antibody 18 and Antibody 18E standard curve, quality control (QC) and test sample dilutions were prepared in 1% cynomolgus monkey plasma and added to blocked plates for 1 hour at room temperature. Plates were washed as above and incubated an additional 1 hour with 1 microg/ml MSD-TAG (Ruthenium)-labeled-Sheep anti-human IgG (H+L) monkey adsorbed detection antibody (The Binding Site). Unbound detection antibody was removed by washing and 150 microl of 1× Read Buffer T (MSD) was added to plate wells. Plates were read immediately with an MSD Sector Imager 2400 and Antibody 18 and Antibody 18E concentrations in QC and test sample dilutions on each plate were quantitated using the standard curve for that plate. All analyses were performed by plotting standard curve concentrations vs. ECL signal in a Log-Log curve fit in Softmax Pro GxP software (Molecular Devices). The assay range for both Antibody 18 and Antibody 18E quantitation is 10,000 to 13.7 ng/ml (10 to 0.0137 microg/ml) in 100% plasma.

PK Data Analysis: Non-compartmental toxicokinetic analysis was performed on individual PK data from all animals using WinNonlin Professional (version 5.2, Pharsight Corp., Mountain View, Calif.), in accordance with MedImmune standard operating procedure.

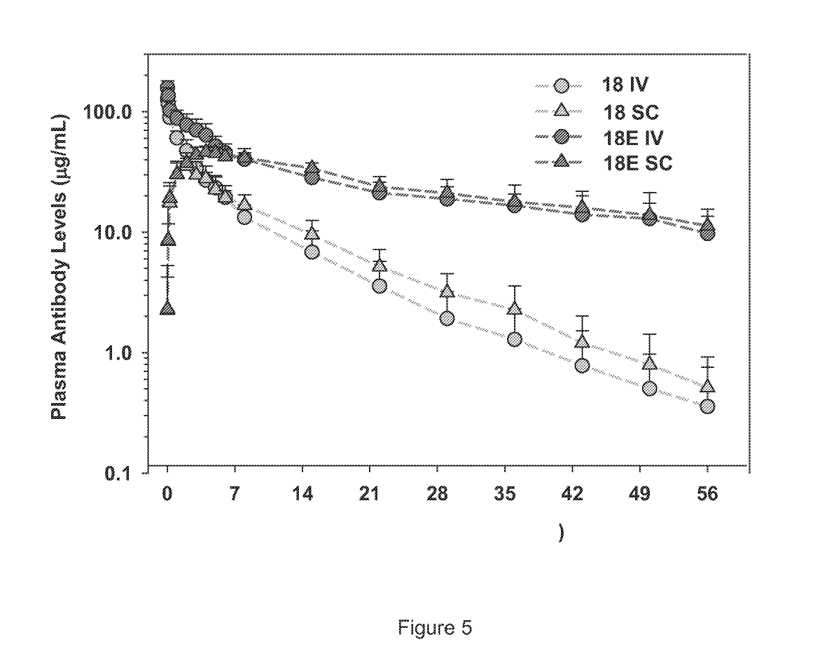

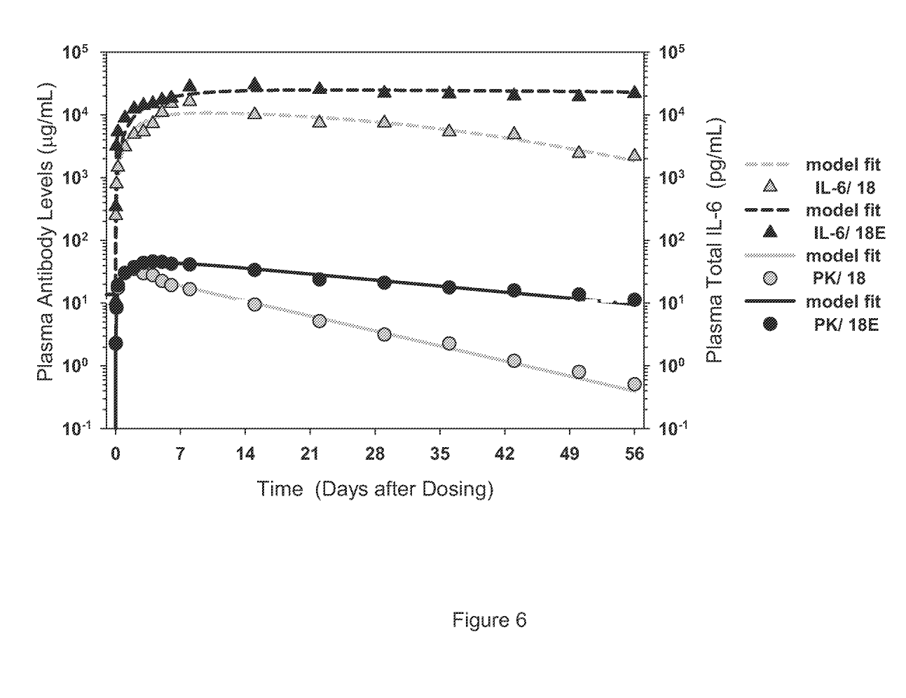

Results obtained are shown in FIGS. 5 and 6. FIG. 5 shows the serum concentration of Antibody 18 and 18E over time following the subcutaneous or intravenous administration of a single 5 mg/ml antibody dose. Both Antibody 18 and 18E exhibited a linear PK profile. The serum half-life of Antibody 18 is approximately 8.5 days and 9.1 days following intravenous and subcutaneous administration, respectively. The serum half-life of Antibody 18E is approximately 28.4 days and 28.8 days following intravenous and subcutaneous administration, respectively. The clearance of Antibody 18 is approximately 12.1 ml/day/kg and 13.1 ml/day/kg following intravenous and subcutaneous administration, respectively. The clearance of Antibody 18E is approximately 2.8 ml/day/kg and 3.0 ml/day/kg following intravenous and subcutaneous administration, respectively. The bioavailability of the subcutaneously administered Antibody 18 and 18E was 94% and 96%, respectively.

FIG. 6 shows the serum concentration of antibody 18 and 18E over time following the subcutaneous administration of a single 5 mg/kg antibody dose. FIG. 6 further shows the total serum IL-6 concentration detected in the animals following the subcutaneous administration of a single 5 mg/kg dose of Antibody 18 or 18E. Total levels of IL-6 increased approximately three logs above baseline. Greater accumulation of total IL-6 was observed with Antibody 18E. The decline in total IL-6 levels approximately paralleled the decline in PK.

### 2.3 Modelling of % Neutralization of Plasma Free IL-6 by Subcutaneously Administered Anti-IL-6 Antibody

The free IL-6 concentrations are very low at baseline in healthy animals. It would have been difficult therefore, to directly assess the % target neutralization following anti-IL-6 antibody administration by measuring free IL-6 levels. We developed a PK/PD model in the SAAM II software package to predict the kinetics of free IL-6 neutralization in relation to antibody PK using total IL-6 as PD marker. The PK/PD model describes the kinetics of antibody, free IL-6, the complex of IL-6 and antibody, the soluble receptor, and the complex of IL-6 and the soluble receptor. The developed model adequately described the PK of antibody and the total IL-6 kinetics generated from the monkey study and it was used to simulate PK/PD time profiles in human RA patients after different dose regimens using standard allometric scaling assumption. 90% inhibition level of human plasma free IL-6 level was set based on serum free IL-6 concentrations detected in rheumatoid arthritis patients (Uson et. al., J. of Rheumatology (1997) 24(11)2069-75).

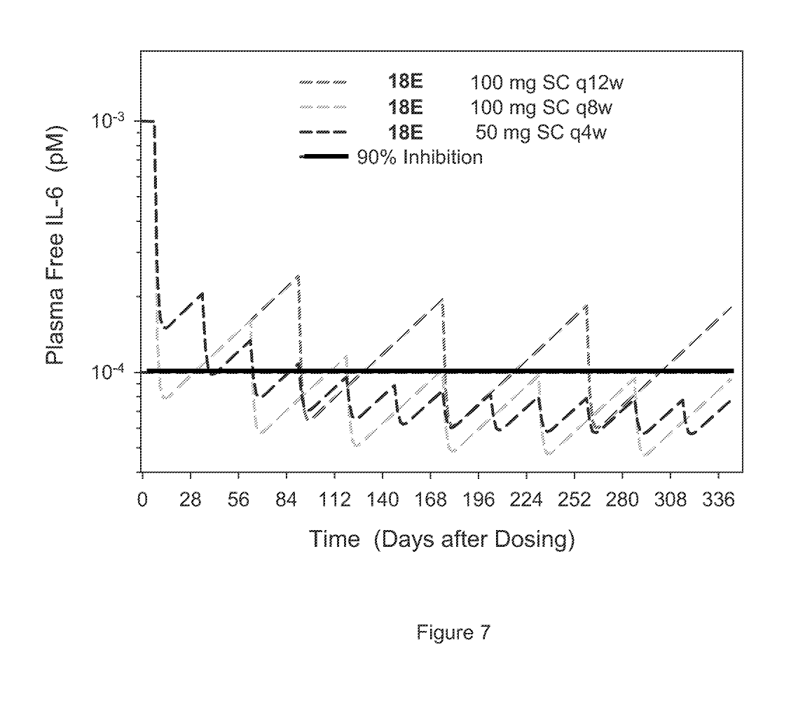

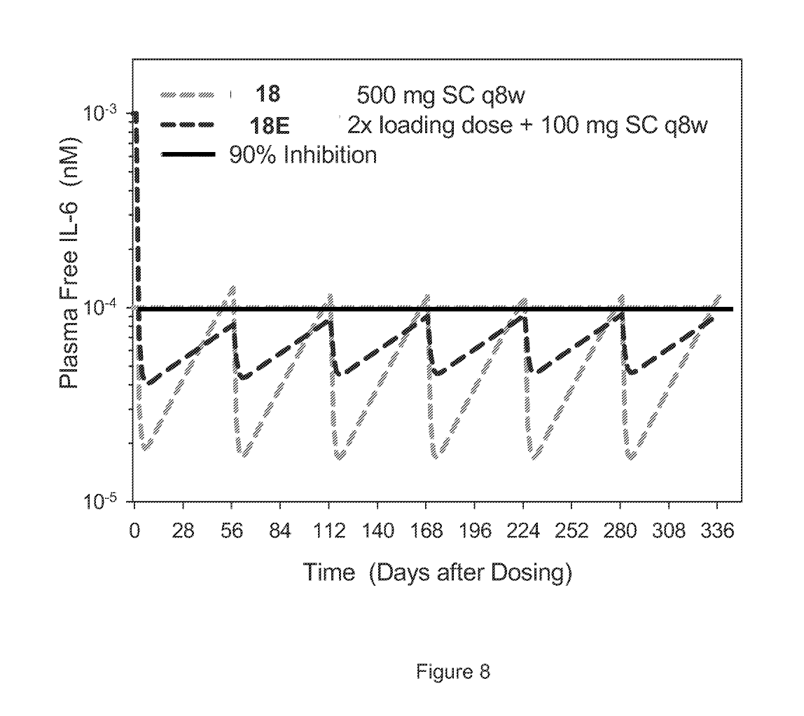

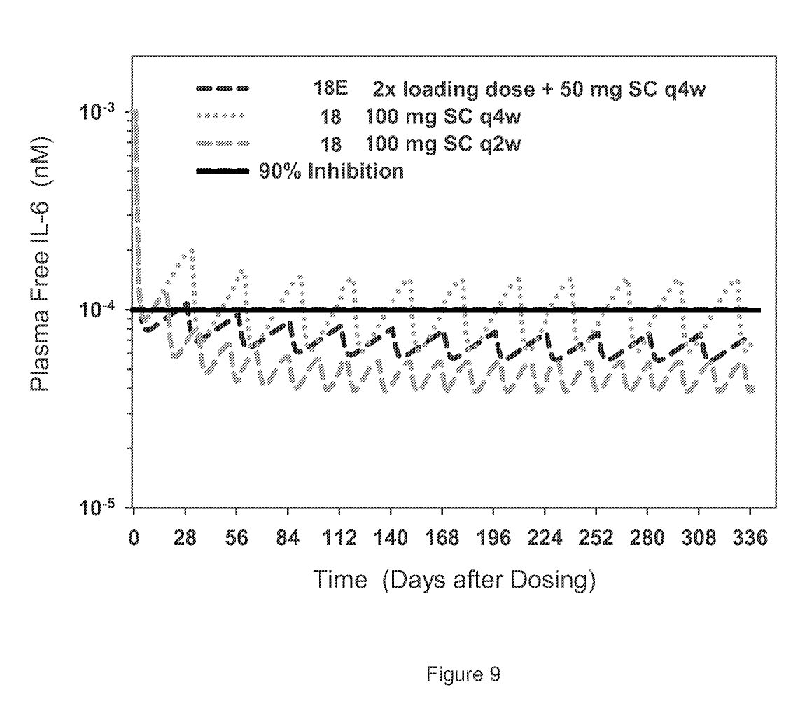

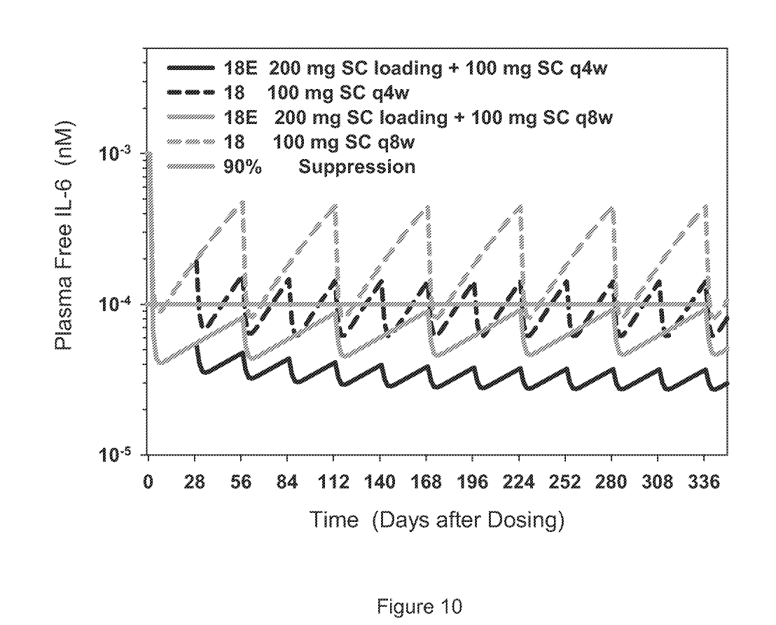

The results of PD modeling are shown in FIGS. 7-10 and Table 3. The model predicts that a sustained at least 90% inhibition of free IL-6 (i.e. not bound to sIL-6R or IL-6R) may be achieved by administering Antibody 18E according to any one of the following dosing regimens:
100 mg Antibody 18E delivered sc every 8 weeks;
50 mg Antibody 18E delivered sc every 4 weeks;
a single loading dose of 200 mg Antibody 18E delivered sc followed by 100 mg Antibody 18E delivered sc every 8 weeks; and
a single loading dose of 100 mg Antibody 18E delivered sc followed by 50 mg Antibody 18E delivered sc every 4 weeks.

The model further predicts that a similar level of continuous inhibition of free serum IL-6 would require more frequent and/or larger doses of Antibody 18. For example, a continuous at least 90% inhibition of free IL-6 (i.e. not bound to sIL-6R or IL-6R) may be achieved by subcutaneously administering 100 mg of Antibody 18 every 2 weeks.

TABLE 3
Summary of results from plasma free IL-6 neutralization model.

| Dosing | | |
|---|---|---|
| interval | Antibody 18 dose | Antibody 18E dose |
| 2 weeks | 100 mg SC | |
| | IL-6 inhibition ≧90% | |
| 4 weeks | 100 mg SC | 50 mg SC or 2x loading dose + |
| | | 50 mg SC maintenance dose |
| | IL-6 inhibition ≦90% | IL-6 inhibition ≧90% |
| 8 weeks | 500 mg SC | 100 mg SC or 2x loading dose + |
| | | 100 mg SC maintenance dose |
| | IL-6 inhibition ≦90% | IL-6 inhibition ≧90% |
| 12 weeks | | 100 mg SC |
| | | IL-6 inhibition ≦90% |

These results demonstrate the ability of an anti-IL-6 antibody to inhibit the systemic effects of IL-6 in vivo. Whereas, particular embodiments of the invention have been described above for purposes of description, it will be appreciated by those skilled in the art that numerous variations of the details may be made without departing from the invention as described in the appended claims.

### Example 3. Efficacy in the Mouse FCA-Induced Inflammatory Pain Model

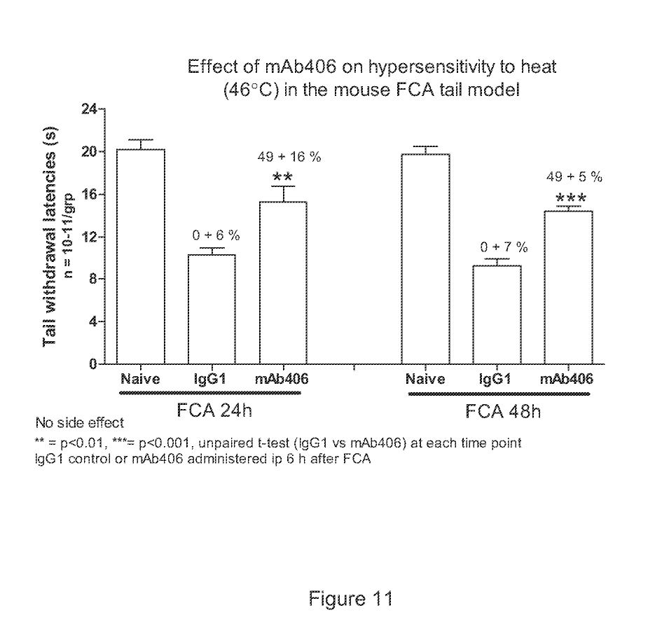

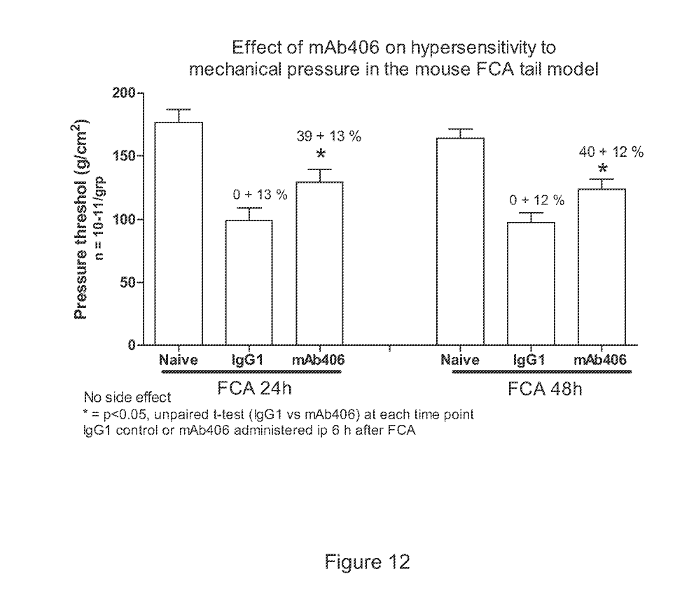

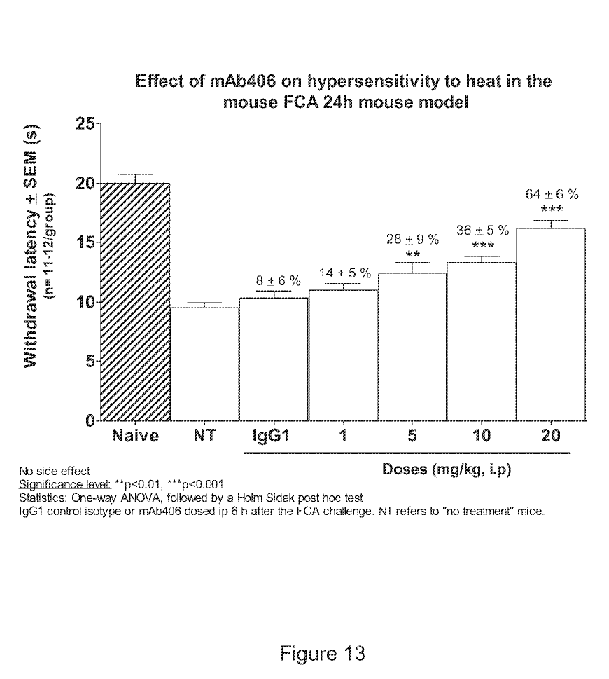

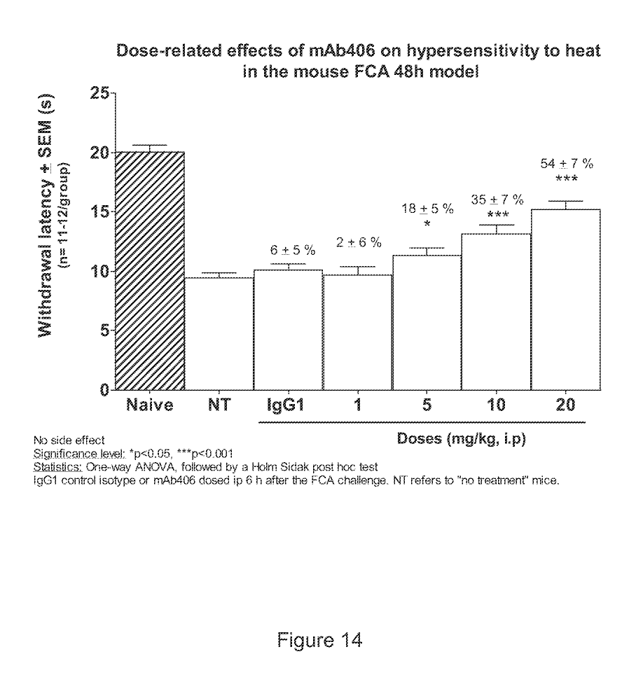

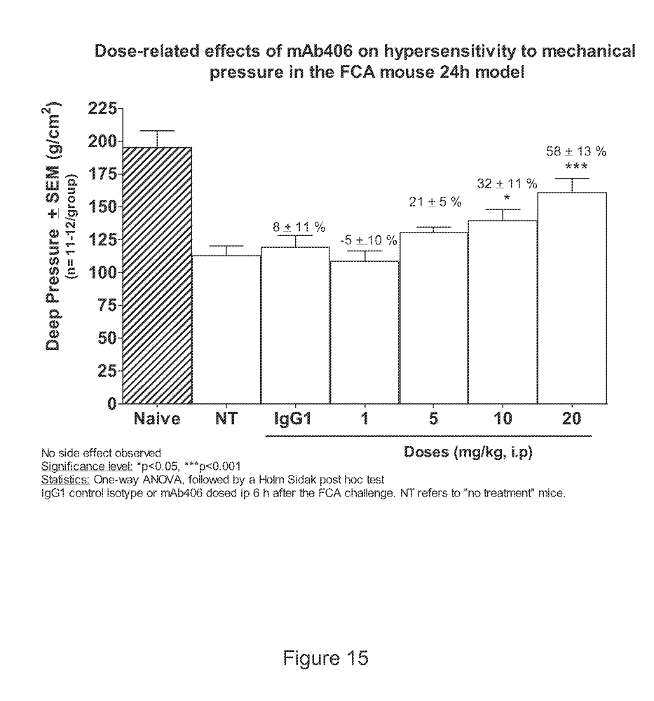

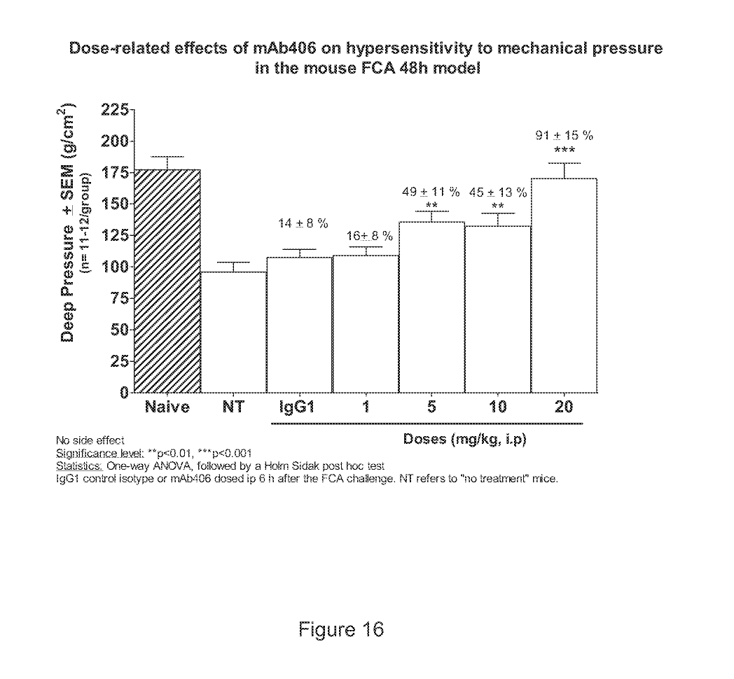

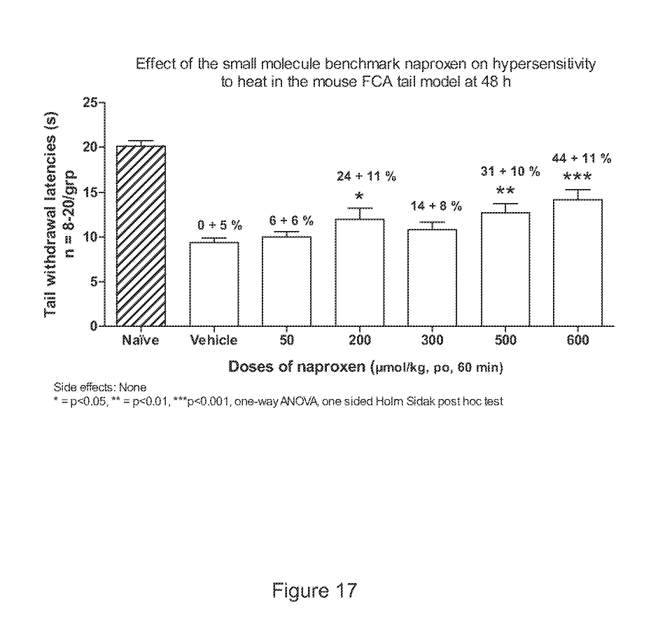

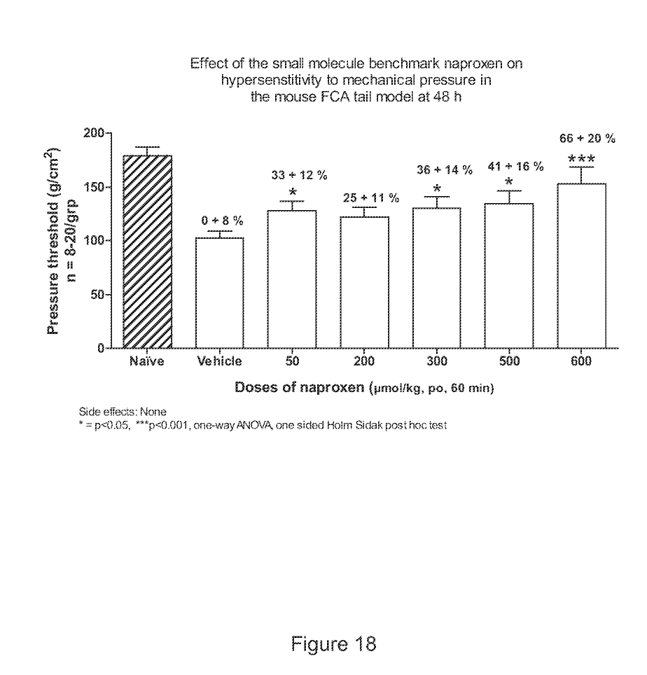

mAb406 (anti mouse IL-6, purified from monoclonal IgG1, clone MP5-20F3, lot AHV100904A, R&D Systems) was tested for its ability to reverse inflammatory pain induced by a local subcutaneous administration of Freund complete adjuvant, (“FCA”) (20 microliters) in the mouse tail (3 cm from the distal tip of the tail). This substance produces a local inflammatory response gradually involving over time and reaching a plateau between 24 h and 48 h after administration. The resulting inflammation produces a hypersensitivity to thermal or mechanical stimulation of the tail. Thermal hyperalgesia is assessed by recording the withdrawal latency of the tail from a thermal stimulus (warm water, 46° C.), while mechanical hyperalgesia is evaluated by the withdrawal threshold of the tail from a steadily increasing pressure generated by an analgesymeter (Randall Selitto apparatus). The IgG1 isotype control (mAb005, purified from rat monoclonal IgG1, clone 43414, lot CAN04904A, purchased from R&D Systems) and mAb406 were administered intraperitoneally (ip) 6 h after FCA treatment. Evidence suggesting that the inflammatory response is well initiated includes elevated cytokine levels, nitric oxide production and hypersensitivity to mildly noxious stimuli. The initial study assessed a single dose (20 mg/kg) of mAb406. This dose produced an E-max of 50% in the heat hyperalgesia assay at both 24 and 48 h (see FIG. 11) and a 40% reversal of mechanical hyperalgesia at 24 and 48 h (see FIG. 12). In vitro profiling of the systemic plasma levels of IL-6 in these animals indicated that all IL-6 had been neutralized. Subsequently, various doses of mAb406 (and a high dose of IgG1 control) were evaluated to characterize the efficacy and IL-6 neutralization with respect to pain inhibition and reversal of hyperalgesia. Using the same experimental paradigm doses ranging from 1 to 20 mg/kg, ip were tested for both heat and mechanical hyperalgesia at both 24 h and 48 h. FIG. 13 shows that heat hyperalgesia was dose-dependently reversed by the anti-IL6 treatment at 24 h and similar results were obtained at 48 h (see FIG. 14). The E-max in this second study was 64% reversal, which is slightly higher but similar to the reversal obtained above. FIG. 15 and FIG. 16 show the results for mechanical hyperalgesia at 24 h and 48 h, respectively. Again a dose-dependent reversal was observed reaching an E-max of 91% at 48 h, which is higher than the reversal obtained in the first study. Side effects were not observed at any of the testing doses and in any of the studies. Overall, the in vivo efficacies with mAb406 were comparable or higher to the benchmarking small molecule compound naproxen in the same model (see FIG. 17 for naproxen in hypersensitivity to heat and to FIG. 18 for naproxen on hypersensitivity to mechanical pressure).

All publications, patents and patent applications mentioned in this specification are herein incorporated by reference into the specification to the same extent as if each individual publication, patent or patent application was specifically and individually indicated to be incorporated herein by reference.

### Materials and Methods

Inhibition of IL-6 Induced Proliferation of TF-1 Cells by Purified scFv and IgG

TF-1 cells were a gift from R&D Systems and maintained according to supplied protocols. Assay media comprised RPMI-1640 with GLUTAMAX I (Invitrogen) containing 5% foetal bovine serum (JRH) and 1% sodium pyruvate (Sigma). Prior to each assay, TF-1 cells were pelleted by centrifugation at 300×g for 5 mins, the media removed by aspiration and the cells re-suspended in assay media. This process was repeated twice with cells re-suspended at a final concentration of 5×10⁵ cells/ml in assay media. The cells were plated out using 100 μl/well in a 96 well assay plate. Plates were incubated for 24 hours at 37° C. and 5% CO₂ to starve cell of GM-CSF. Test solutions of purified scFv or IgG (in duplicate) were diluted to the desired concentration in assay media. An irrelevant antibody not directed at IL-6 was used as negative control. Recombinant bacterially derived human (R&D) and cynomolgus (in-house) IL-6 was added to a final concentration of either 20 pM (human IL-6) or 100 pM (cynomolgus) when mixed with appropriate test antibody in a total volume of 100 μl/well. The concentration of IL-6 used in the assay was selected as the dose that at final assay concentration gave approximately 80% of maximal proliferative response. All samples were incubated for 30 mins at room temperature. 100 μl of IL-6 and antibody mixture was then added to 100 μl of the cells to give a total assay volume of 200 μl/well. Plates were incubated for 24 hours at 37° C. and 5% CO₂. 20 μl of tritiated thymidine (5 μCi/ml) was then added to each assay point and the plates were returned to the incubator for further 24 hours. Cells were harvested on glass fibre filter plates (Perkin Elmer) using a cell harvester. Thymidine incorporation was determined using Packard TopCount microplate liquid scintillation counter. Data was then analysed using Graphpad Prism software.

Inhibition of Endogenous IL-6 Induced VEGF Release from Human Synovial Fibroblasts by Purified IgG

Samples of rheumatoid arthritis knees from total joint replacement surgery were obtained in DMEM containing antibiotics. Synovium bathed in media was dissected from the joint & finely chopped. The synovial tissue was washed with media supplemented with 10% FCS. The cell suspension was incubated in a collagenase solution for 2 hours in a CO₂ incubator at 37° C. The digested synovial cell suspension was disrupted by repeatedly aspirating through a 10 ml pipette, cell strained & centrifuged at 400 g at room temperature for 5 minutes. The cells were resuspended in DMEM containing 10% FCS, passed through a cell strainer, adjusted to 1×10⁶ cells per ml & incubated in a CO₂ incubator at 37° C. in 225-cm² cell culture flasks (3001, CoStar Corning Inc.). Following adherence, the majority of the medium was discarded, replaced with fresh & returned to the incubator for long-term incubation. The cells were examined on a weekly-basis & were passaged at confluence by trypsinisation at a passage rate of 1 in 3.

Fibroblasts (P3-5) at confluence were removed from flasks by incubating with 10 mL 0.1% trypsin-EDTA solution (25300-054, Gibco Life Sciences) per flask for 5 to 10 minutes at 37° C. An equal volume of DMEM-based culture medium supplemented with 10% FCS was added to the cells, which were then pelleted by centrifugation at 330 g for 5 minutes at RT. After one wash step with DMEM-based culture medium supplemented with 10% FCS, the cell suspension (1×10⁵ cells per mL) was added (150 μL per well) to wells of sterile 96 well cell culture cluster flat bottom polystyrene plates (3598, Corning CoStar) at 1.5×10⁴ cells per well. A further addition of DMEM-based culture media supplemented with 10% FCS was added to each well (100 μL per well) to give a total volume of 250 μL per well. The cells were incubated at 37° C. overnight to allow for adherence and quiescence.

The 96-well plates were inspected to ensure that the cells were confluent and in good condition (e.g. contamination-free). Medium was then aspirated from the wells and 100 μL of DMEM-based culture medium supplemented with 10% FCS was immediately added. To this, 50 μL of DMEM-based culture medium supplemented with 10% FCS containing either sample IgG or medium alone was added to the wells (diluted 1 in 5 into assay).

This was followed by adding 50 μL per well of DMEM-based culture medium supplemented with 10% FCS containing recombinant human soluble (rhs)IL-6Rα (500 ng per mL; 12 nM) and rhIL-1β (50 pg per mL; 2.95 pM, diluted 1 in 5 into assay).

In separate wells, 50 μL of DMEM-based culture medium supplemented with 10% FCS containing either; rh-IL-6 (0, 100 ng per mL; 21.5 nM), sIL-6Rα (500 ng per mL; 12 nM), rhIL-1β (50 pg per mL; 2.95 pM), or medium alone was added (diluted 1 in 5 into assay). Final volume in each well was 250 μL.

The plates were incubated for 48 hours at 37° C. Incubations were performed in duplicate or triplicate wells as described in the plate format. The plates were centrifuged at 330 g for 5 minutes at RT and supernatant media was removed and stored at −40° C. in microlitre flat bottom plates (611F96, Sterilin).

VEGF was measured using an ELISA (DY293B, R&D Systems) following the manufacturers instructions. Briefly, ELISA plates were coated with a mouse anti-human VEGF antibody overnight at 4° C. and blocked with 1% BSA/PBS. Plates were washed with 0.05% Tween 20/PBS and incubated with culture supernatants of human synovial derived fibroblasts and a biotinylated goat anti-human VEGF antibody over night at room temperature. After washing, VEGF was detected by using Streptavidin horseradish peroxidase. Plates were developed using 1:1 H₂O₂:tetramethylbenzidine. The reaction was stopped with 2 M H₂SO₄, and optical densities were determined at 450 nm with the correction wavelength set at 540 nm.

### Measurement of Total Plasma IL-6 Levels

Total IL-6 is measured using the Milliplex™ MAP kit (MPXHCYTO60K) according to the manufacturer's recommendations. All necessary reagents are provided in the assay kit. Briefly, an assay filter plate is hydrated with 200 μL of assay buffer and the liquid is removed by vacuum. Each of the following reagents is added to the plate at 25 μL/well: (a) assay buffer (b) plasma samples, standards or QC and (c) beads conjugated with an anti-IL-6 capture antibody in assay matrix. The final assay volume is 75 and the final assay matrix is 133.3 μg/ml drug candidate (Antibody 18 or Antibody 18E) in 25% of IL-6 depleted normal cyno EDTA plasma. The plate is sealed and incubated for overnight at 4° C. After 2 washes with wash buffer, 25 μl/well of biotinylated anti-IL6 detection antibody is added. After 30 minutes incubation, 25 μL/well of SA-PE is added to the wells and the plate is incubated for an additional 30 minutes. The plate is washed twice and the beads are re-suspended with 150 μl/well of Luminex Sheath Fluid. Fluorescence intensity associated with the beads is measured by the Luminex200 Plate reader. Since both capture and detection anti-IL-6 antibodies are able to bind to IL-6 in the presence of Antibody 18 or Antibody 18E, the fluorescent intensity is proportional to the total IL-6 concentration in the sample. The concentration of IL-6 is extrapolated from a standard curve plotted with the BeadView Software (Upstate Cell Signaling Solutions). The detection range for IL-6 is 1.8 pg/ml to 5769 pg/ml in 100% cyno plasma.

All references cited anywhere in this specification, including those cited anywhere above, are incorporated herein by reference in their entirety and for all purposes.

Sequences
SEQ ID NO: 1; Ab 18 VH CDR1
SNYMI
SEQ ID NO: 2; Ab 18 VH CDR2
DLYYYAGDTYYADSVKG
SEQ ID NO: 3; Ab 18 VH CDR3
WADDHPPWIDL
SEQ ID NO: 4; Ab 18 VL CDR1
RASQGISSWLA
SEQ ID NO: 5 Ab 18 VL CDR2
KASTLES
SEQ ID NO: 6 Ab 18 VL CDR3.
QQSWLGGS
SEQ ID NO: 7 Ab 18 VH
EVQLVESGGGLVQPGGSLRLSCAASGFTISSNYMIWVRQAPGKGLEWVSDLYYYAGDTYYADSVK
GRFTMSRDISKNTVYLQMNSLRAEDTAVYYCARWADDHPPWIDLWGRGTLVTVSS
SEQ ID NO: 8 Ab 18 VL
DIQMTQSPSTLSASVGDRVTITCRASQGISSWLAWYQQKPGKAPKVLIYKASTLESGVPSRFSGSGSG
TEFTLTISSLQPDDFATYYCQQSWLGGSFGQGTKLEIK
SEQ ID NO: 9 Ab 18E HC
EVQLVESGGGLVQPGGSLRLSCAASGFTISSNYMIWVRQAPGKGLEWVSDLYYYAGDTYYADSVK
GRFTMSRDISKNTVYLQMNSLRAEDTAVYYCARWADDHPPWIDLWGRGTLVTVSSASTKGPSVFPL
APSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLYITREPEVTCVVV
DVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 10 Ab 18E LC
DIQMTQSPSTLSASVGDRVTITCRASQGISSWLAWYQQKPGKAPKVLIYKASTLESGVPSRFSGSGSG
TEFTLTISSLQPDDFATYYCQQSWLGGSFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSP
VTKSFNRGEC
SEQ ID NO: 11 Ab 18 optimized VH
GAGGTGCAGCTGGTCGAGTCTGGCGGCGGACTGGTGCAGCCTGGCGGCTCCCTGCGGCTGTCCT
GCGCCGCCTCCGGCTTCACCATCTCCTCCAACTACATGATTTGGGTCCGCCAGGCACCTGGCAAG
GGGCTCGAGTGGGTGTCCGACCTGTACTACTACGCCGGCGACACCTACTACGCTGACTCCGTGA
AGGGCCGGTTCACCATGTCCAGGGACATCTCCAAGAACACCGTGTACCTGCAGATGAACTCCCT
GCGGGCCGAGGACACCGCCGTGTACTACTGCGCCAGATGGGCCGACGACCACCCTCCTTGGATC
GACCTGTGGGGCAGGGGCACCCTGGTCACCGTCAGCTCC
SEQ ID NO: 12 Ab 18 optimized VL
GACATCCAGATGACCCAGTCCCCCTCCACCCTGTCCGCCAGCGTCGGCGACAGAGTGACCATCA
CCTGCCGGGCCTCCCAGGGCATCTCCAGCTGGCTGGCCTGGTATCAGCAGAAGCCTGGCAAGGC
CCCTAAGGTGCTGATCTACAAGGCCAGCACCCTGGAGTCCGGCGTGCCTTCCCGGITCTCCGGCT
CCGGCAGCGGCACCGAGTTCACCCTGACCATCTCCTCCCTGCAGCCTGACGACTTCGCCACCTAC
TACTGCCAGCAGTCCTGGCTGGGCGGCTCCTTCGGCCAGGGCACCAAGCTGGAGATCAAG
SEQ ID NO: 13 Ab 18E optimized HC
GAGGTGCAGCTGGTCGAGTCTGGCGGCGGACTGGTGCAGCCTGGCGGCTCCCTGCGGCTGTCCT
GCGCCGCCTCCGGCTTCACCATCTCCTCCAACTACATGATTTGGGTCCGCCAGGCACCTGGCAAG
GGGCTCGAGTGGGTGTCCGACCTGTACTACTACGCCGGCGACACCTACTACGCTGACTCCGTGA
AGGGCCGGTTCACCATGTCCAGGGACATCTCCAAGAACACCGTGTACCTGCAGATGAACTCCCT
GCGGGCCGAGGACACCGCCGTGTACTACTGCGCCAGATGGGCCGACGACCACCCTCCTTGGATC
GACCTGTGGGGCAGGGGCACCCTGGTCACCGTCAGCTCCGCCTCCACCAAGGGCCCCAGCGTGT
TCCCCCTGGCCCCCAGCAGCAAGAGCACCTCCGGCGGCACAGCCGCCCTGGGCTGCCTGGTGAA
GGACTACTTCCCCGAACCGGTGACCGTGTCCTGGAACAGCGGCGCTCTGACCAGCGGCGTGCAC
ACCTTCCCCGCCGTGCTGCAGAGCAGCGGCCTGTACAGCCTGAGCAGCGTGGTGACAGTGCCCA
GCAGCAGCCTGGGCACCCAGACCTACATCTGCAACGTGAACCACAAGCCCAGCAACACCAAGG
TGGACAAGAGAGTGGAGCCCAAGAGCTGCGACAAGACCCACACCTGCCCCCCCTGCCCTGCCCC
TGAGCTGCTGGGCGGACCTAGCGTGTTCCTGTTCCCCCCCAAGCCCAAGGACACCCTGTACATCA
CCAGGGAGCCCGAGGTGACCTGCGTGGTGGTGGACGTGAGCCACGAGGACCCTGAGGTGAAGT
TCAATTGGTACGTGGACGGCGTGGAGGTGCACAACGCCAAGACCAAGCCCAGAGAGGAGCAGT
ACAACAGCACCTACAGGGTGGTGTCCGTGCTGACCGTGCTGCACCAGGACTGGCTGAACGGCAA
GGAATACAAGTGCAAGGTCTCCAACAAGGCCCTGCCTGCCCCCATCGAAAAGACCATCAGCAAG
GCCAAGGGCCAGCCTCGGGAGCCCCAGGTGTACACCCTGCCCCCTAGCCGGGAGGAGATGACC
AAGAACCAGGTGTCCCTGACCTGTCTGGTGAAGGGCTTCTACCCCAGCGACATCGCCGTGGAGT
GGGAGAGCAACGGCCAGCCCGAGAACAACTACAAGACCACCCCCCCTGTGCTGGACAGCGACG
GCAGCTTCTTCCTGTACAGCAAGCTGACCGTGGACAAGTCCAGGTGGCAGCAGGGCAACGTGTT
CAGCTGCAGCGTGATGCACGAGGCCCTGCACAACCACTACACCCAGAAGAGCCTGAGCCTGTCC
CCCGGCAAG
SEQ ID NO: 14 Ab 18E optimized LC
GACATCCAGATGACCCAGTCCCCCTCCACCCTGTCCGCCAGCGTCGGCGACAGAGTGACCATCA
CCTGCCGGGCCTCCCAGGGCATCTCCAGCTGGCTGGCCTGGTATCAGCAGAAGCCTGGCAAGGC
CCCTAAGGTGCTGATCTACAAGGCCAGCACCCTGGAGTCCGGCGTGCCTTCCCGGTTCTCCGGCT
CCGGCAGCGGCACCGAGTTCACCCTGACCATCTCCTCCCTGCAGCCTGACGACTTCGCCACCTAC
TACTGCCAGCAGTCCTGGCTGGGCGGCTCCTTCGGCCAGGGCACCAAGCTGGAGATCAAGCGTA
CGGTGGCCGCTCCCAGCGTGTTCATCTTCCCCCCCAGCGACGAGCAGCTGAAGAGCGGCACCGC
CTCCGTGGTGTGCCTGCTGAACAACTTCTACCCCCGCGAGGCCAAGGTGCAGTGGAAGGTGGAC
AACGCCCTGCAGTCCGGCAACAGCCAGGAGAGCGTCACCGAGCAGGACAGCAAGGACTCCACC
TACAGCCTGAGCAGCACCCTGACCCTGAGCAAGGCCGACTACGAGAAGCACAAGGTGTACGCCT
GCGAGGTGACCCACCAGGGCCTGTCCAGCCCCGTGACCAAGAGCTTCAACAGGGGCGAGTGC
SEQ ID NO: 15 Human IL-6
MNSFSTSAFGPVAFSLGLLLVLPAAFPAPVPPGEDSKDVAAPHRQPLTSSERIDKQIRYILDGISALRKETCN
KSNMCESSKEALAENNLNLPKMAEKDGCFQSGFNEETCLVKIITGLLEFEVYLEYLQNRFESSEEQARAVQMS
TKVLIQFLQKKAKNLDAITTPDPTTNASLLTKLQAQNQWLQDMTTHLILRSFKEFLQSSLRALRQM
SEQ ID NO: 16 Mature human IL-6
VPPGEDSKDVAAPHRQPLTSSERIDKQIRYILDGISALRKETCNKSNMCESSKEALAENNLNLPKMAEKDGCF
QSGFNEETCLVKIITGLLEFEVYLEYLQNRFESSEEQARAVQMSTKVLIQFLQKKAKNLDAITTPDPTTNASL
LTKLQAQNQWLQDMTTHLILRSFKEFLQSSLRALRQM
SEQ ID NO: 17 sIL-6Ra
MLAVGCALLAALLAAPGAALAPRRCPAQEVARGVLTSLPGDSVTLTCPGVEPEDNATVHWVLRKPAAGSHPSR
WAGMGRRLLLRSVQLHDSGNYSCYRAGRPAGTVHLLVDVPPEEPQLSCFRKSPLSNVVCEWGPRSTPSLTTKA
VLLVRKFQNSPAEDFQEPCQYSQESQKFSCQLAVPEGDSSFYIVSMCVASSVGSKFSKTQTFQGCGILQPDPP
ANITVTAVARNPRWLSVTWQDPHSWNSSFYRLRFELRYRAERSKTFTTWMVKDLQHHCVIHDAWSGLRHVVQL
RAQEEFGQGEWSEWSPEAMGTPWTESRSPPAENEVSTPMQALTTNKDDDNILFRDSANATSLPVQD
SEQ ID NO: 18 Transmembrane domain of IL-6Ra
MLAVGCALLAALLAAPGAALAPRRCPAQEVARGVLTSLPGDSVTLTCPGVEPEDNATVHWVLRKPAAGSHPSR
WAGMGRRLLLRSVQLHDSGNYSCYRAGRPAGTVHLLVDVPPEEPQLSCFRKSPLSNVVCEWGPRSTPSLTTKA
VLLVRKFQNSPAEDFQEPCQYSQESQKFSCQLAVPEGDSSFYIVSMCVASSVGSKFSKTQTFQGCGILQPDPP
ANITVTAVARNPRWLSVTWQDPHSWNSSFYRLRFELRYRAERSKTFTTWMVKDLQHHCVIHDAWSGLRHVVQL
RAQEEFGQGEWSEWSPEAMGTPWTESRSPPAENEVSTPMQALTTNKDDDNILFRDSANATSLPVQDSSSVPLP
TFLVAGGSLAFGTLLCIAIVLRFKKTWKLRALKEGKTSMHPPYSLGQLVPERPRPTPVLVPLISPPVSPSSLG
SDNTSSHNRPDARDPRSPYDISNTDYFFPR
SEQ ID NO: 19 Gp130
MLTLQTWLVQALFIFLTTESTGELLDPCGYISPESPVVQLHSNFTAVCVLKEKCMDYFHVNANYIVWKTNHFT
IPKEQYTIINRTASSVTFTDIASLNIQLTCNILTFGQLEQNVYGITIISGLPPEKPKNLSCIVNEGKKMRCEW
DGGRETHLETNFTLKSEWATHKFADCKAKRDTPTSCTVDYSTVYFVNIEVWVEAENALGKVTSDHINFDPVYK
VKPNPPHNLSVINSEELSSILKLTWTNPSIKSVIILKYNIQYRTKDASTWSQIPPEDTASTRSSFTVQDLKPF
TEYVFRIRCMKEDGKGYWSDWSEEASGITYEDRPSKAPSFWYKIDPSHTQGYRTVQLVWKTLPPFEANGKILD
YEVTLTRWKSHLQNYTVNATKLTVNLTNDRYLATLTVRNLVGKSDAAVLTIPACDFQATHPVMDLKAFPKDNM
LWVEWTTPRESVKKYILEWCVLSDKAPCITDWQQEDGTVHRTYLRGNLAESKCYLITVTPVYADGPGSPESIK
AYLKQAPPSKGPTVRTKKVGKNEAVLEWDQLPVDVQNGFIRNYTIFYRTIIGNETAVNVDSSHTEYTLSSLTS
DTLYMVRMAAYTDEGGKDGPEFTFTTPKFAQGEIEAIVVPVCLAFLLTTLLGVLFCFNKRDLIKKHIWPNVPD
PSKSHIAQWSPHTPPRHNFNSKDQMYSDGNFTDVSVVEIEANDKKPFPEDLKSLDLFKKEKINTEGHSSGIGG
SSCMSSSRPSISSSDENESSQNTSSTVQYSTVVHSGYRHQVPSVQVFSRSESTQPLLDSEERPEDLQLVDHVD
GGDGILPRQQYFKQNCSQHESSPDISHFERSKQVSSVNEEDFVRLKQQISDHISQSCGSGQMKMFQEVSAADA
FGPGTEGQVERFETVGMEAATDEGMPKSYLPQTVRQGGYMPQ

What is claimed is:

1. An isolated antibody that specifically binds to IL-6, wherein the antibody comprises the amino acid sequences of SEQ ID NO: 9 and SEQ ID NO: 10.

2. An isolated nucleic acid encoding the amino acid sequences of claim 1.

3. The nucleic acid of claim 2 comprising the nucleotide sequence of SEQ ID NO: 13 and SEQ ID NO: 14.

4. An isolated vector comprising the nucleic acid of claim 3.

5. An isolated cell comprising the vector of claim 4.

6. An isolated cell line expressing the antibody of claim 1.

7. A pharmaceutical composition comprising the antibody of claim 1 in a pharmaceutically acceptable carrier.


SEQUENCE LISTING (19 sequences)

SEQ ID NO: 1 - protein, 5 aa, Artificial Sequence
  Ab 18 VH CDR1
SNYMI

SEQ ID NO: 2 - protein, 17 aa, Artificial
  Ab 18 VH CDR2
DLYYYAGDTYYADSVKG

SEQ ID NO: 3 - protein, 11 aa, Artificial
  Ab 18 VH CDR3
WADDHPPWIDL

SEQ ID NO: 4 - protein, 11 aa, Artificial
  Ab 18 VL CDR1
RASQGISSWLA

SEQ ID NO: 5 - protein, 7 aa, Artificial
  Ab 18 VL CDR2
KASTLES

SEQ ID NO: 6 - protein, 8 aa, Artificial
  Ab 18 VL CDR3
QQSWLGGS

SEQ ID NO: 7 - protein, 120 aa, Artificial
  Ab 18 VH
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSS

SEQ ID NO: 8 - protein, 106 aa, Artificial
  Ab 18 VL
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIK

SEQ ID NO: 9 - protein, 450 aa, Artificial
  Ab 18E HC
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial
  Ab 18E LC
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 360 bp, Artificial
  Ab 18 optimized VH
gaggtgcagctggtcgagtctggcggcggactggtgcagcctggcggctccctgcggctgtcctgcgccgcctccggcttcaccatctcctccaactacatgatttgggtccgccaggcacctggcaaggggctcgagtgggtgtccgacctgtactactacgccggcgacacctactacgctgactccgtgaagggccggttcaccatgtccagggacatctccaagaacaccgtgtacctgcagatgaactccctgcgggccgaggacaccgccgtgtactactgcgccagatgggccgacgaccaccctccttggatcgacctgtggggcaggggcaccctggtcaccgtcagctcc

SEQ ID NO: 12 - DNA, 318 bp, Artificial
  Ab 18 optimized VL
gacatccagatgacccagtccccctccaccctgtccgccagcgtcggcgacagagtgaccatcacctgccgggcctcccagggcatctccagctggctggcctggtatcagcagaagcctggcaaggcccctaaggtgctgatctacaaggccagcaccctggagtccggcgtgccttcccggttctccggctccggcagcggcaccgagttcaccctgaccatctcctccctgcagcctgacgacttcgccacctactactgccagcagtcctggctgggcggctccttcggccagggcaccaagctggagatcaag

SEQ ID NO: 13 - DNA, 1350 bp, Artificial
  Ab 18E optimized HC
gaggtgcagctggtcgagtctggcggcggactggtgcagcctggcggctccctgcggctgtcctgcgccgcctccggcttcaccatctcctccaactacatgatttgggtccgccaggcacctggcaaggggctcgagtgggtgtccgacctgtactactacgccggcgacacctactacgctgactccgtgaagggccggttcaccatgtccagggacatctccaagaacaccgtgtacctgcagatgaactccctgcgggccgaggacaccgccgtgtactactgcgccagatgggccgacgaccaccctccttggatcgacctgtggggcaggggcaccctggtcaccgtcagctccgcctccaccaagggccccagcgtgttccccctggcccccagcagcaagagcacctccggcggcacagccgccctgggctgcctggtgaaggactacttccccgaaccggtgaccgtgtcctggaacagcggcgctctgaccagcggcgtgcacaccttccccgccgtgctgcagagcagcggcctgtacagcctgagcagcgtggtgacagtgcccagcagcagcctgggcacccagacctacatctgcaacgtgaaccacaagcccagcaacaccaaggtggacaagagagtggagcccaagagctgcgacaagacccacacctgccccccctgccctgcccctgagctgctgggcggacctagcgtgttcctgttcccccccaagcccaaggacaccctgtacatcaccagggagcccgaggtgacctgcgtggtggtggacgtgagccacgaggaccctgaggtgaagttcaattggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgcctgcccccatcgaaaagaccatcagcaaggccaagggccagcctcgggagccccaggtgtacaccctgccccctagccgggaggagatgaccaagaaccaggtgtccctgacctgtctggtgaagggcttctaccccagcgacatcgccgtggagtgggagagcaacggccagcccgagaacaactacaagaccaccccccctgtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 14 - DNA, 639 bp, Artificial
  Ab 18E optimized LC
gacatccagatgacccagtccccctccaccctgtccgccagcgtcggcgacagagtgaccatcacctgccgggcctcccagggcatctccagctggctggcctggtatcagcagaagcctggcaaggcccctaaggtgctgatctacaaggccagcaccctggagtccggcgtgccttcccggttctccggctccggcagcggcaccgagttcaccctgaccatctcctccctgcagcctgacgacttcgccacctactactgccagcagtcctggctgggcggctccttcggccagggcaccaagctggagatcaagcgtacggtggccgctcccagcgtgttcatcttcccccccagcgacgagcagctgaagagcggcaccgcctccgtggtgtgcctgctgaacaacttctacccccgcgaggccaaggtgcagtggaaggtggacaacgccctgcagtccggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgc

SEQ ID NO: 15 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 16 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 17 - protein, 358 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQD

SEQ ID NO: 18 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 19 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ